(19) United States
(12) Patent Application Publication
(10) Pub. No.: US 2023/0175072 A1
(43) Pub. Date: Jun. 8, 2023

(54) 3 FLAP OKAZAKI FRAGMENTS AND USES THEREOF

(71) Applicant: City of Hope, Duarte, CA (US)

(72) Inventors: Li Zheng, Arcadia, CA (US); Binghui Shen, La Verne, CA (US); Haitao Sun, Duarte, CA (US); Mian Zhou, Duarte, CA (US)

(21) Appl. No.: 18/073,397

(22) Filed: Dec. 1, 2022

Related U.S. Application Data
(60) Provisional application No. 63/285,437, filed on Dec. 2, 2021.

(51) Int. Cl. C12Q 1/6886

(52) U.S. Cl. CPC C12Q 1/6886; C12Q 2600/156; C12Q 2600/158

(57) ABSTRACT

Provided herein, inter alia, are methods of detecting 3′ flap Okazaki fragments for treating and diagnosing cancer.

### CROSS-REFERENCE TO RELATED APPLICATIONS

This application claims the benefit of priority to U.S. Application No. 63/285,437 filed Dec. 2, 2021, the disclosure of which is incorporated by reference herein in its entirety.

### STATEMENT AS TO RIGHTS TO INVENTIONS MADE UNDER FEDERALLY SPONSORED RESEARCH AND DEVELOPMENT

This invention was made with government support under grant R01 CA073764, R50 CA211397, and R01 CA065344 awarded by The National Institutes of Health. The government has certain rights in the invention.

### REFERENCE TO A “SEQUENCE LISTING,” A TABLE, OR A COMPUTER PROGRAM LISTING APPENDIX SUBMITTED AS AN ASCII FILE

The Sequence Listing written in file X, created on X, having X bytes, machine format IBM-PC, MS Windows operating system, is hereby incorporated by reference.

### BACKGROUND

Understanding the mutagenesis mechanisms that are active in cells under stress conditions is crucial for developing strategies to intervene in microbial pathogenesis, tumorigenesis, and drug resistance (1, 2). Lagging-strand DNA synthesis is particularly vulnerable to stress and environmental factors. During replication, lagging-strand DNA is synthesized as discrete Okazaki fragments (3), which contain short primase/Polα-synthesized RNA-DNA primers at their 5′ ends (4-6). During Okazaki fragment maturation (OFM), the RNA portion and any Polα-synthesized DNA with high incorporation errors are removed, via Polo-mediated displacement DNA synthesis, which produces a 5′ RNA-DNA flap (4-6). The 5′ flap structure is removed by flap endonuclease 1 (FEN1) or through the sequential actions of DNA2 nuclease/helicase and FEN1 (7-9). FEN1 deficiency leads to accumulation of unprocessed 5′ flap structures, which may prevent ligation of Okazaki fragments, leaving DNA nicks or gaps that lead to collapse of replication forks and DNA double-strand breaks. The disclosure addresses these problems

### BRIEF SUMMARY

Provided herein are methods for treating cancer in a patient in need thereof by administering to the patient a therapeutically effective amount of a DNA Damage Response Inhibitor; wherein a biological sample obtained from the patient contains a 3′ flap Okazaki fragment. In embodiments, the DNA Damage Response Inhibitor is an ataxia-telangiectasia mutated (ATM) kinase inhibitor, an ataxia telangiectasia and Rad3-related (ATR) kinase inhibitor, a checkpoint kinase 1 (Chk1) kinase inhibitor, a checkpoint kinase 2 (Chk2) kinase inhibitor, or a combination of two or more thereof. In embodiments, the methods further comprise administering to the patient an anticancer agent, such as a cytotoxic agent.

Provided herein are methods for treating cancer in a patient in need thereof, the method comprising: (i) detecting a 3′ flap Okazaki fragment in a biological sample obtained from the patient; and (ii) administering to the patient a therapeutically effective amount of a DNA Damage Response Inhibitor. In embodiments, the DNA Damage Response Inhibitor is an ATM kinase inhibitor, an ATR kinase inhibitor, a Chk1 kinase inhibitor, a Chk2 kinase inhibitor, or a combination of two or more thereof. In embodiments, the methods further comprise administering to the patient an anticancer agent, such as a cytotoxic agent.

Provided herein are methods for identifying a cancer patient in need of treatment with a DNA Damage Response Inhibitor, the method comprising detecting a 3′ flap Okazaki fragment in a biological sample obtained from the patient; wherein the presence of the 3′ flap Okazaki fragment indicates that the cancer patient is in need of treatment with the DNA Damage Response Inhibitor.

Provided herein are methods for diagnosing a patient with cancer, the method comprising detecting a 3′ flap Okazaki fragment in a biological sample obtained from the patient; wherein the presence of the 3′ flap Okazaki fragment indicates that the patient has cancer.

### BRIEF DESCRIPTION OF THE DRAWINGS

FIGS. 1A-1C. Polδ internal tandem duplication (ITD) and missense mutations drive resistance to rad27A-induced conditional lethality. FIG. 1A: Spot assays of WT, rad27Δ, or rad27Δ revertant (Rev) yeast cells grown at 30° C. (optimal temperature), 25° C. (sub-optimal temperature), or 37° C.C (restrictive temperature) for 48 h. rad27Δ::URA3 and rad27Δ::LEU2 represent the rad27Δ allele with a linked URA3 or LEU2 selection marker gene, respectively. FIG. 1B: pol3 mutations detected in independent revertant strains (n=31). Circles and diamonds represent base substitution and ITD mutations, respectively. The domain structures were defined as previously described (23). FIG. 1C: Spot assays of WT, rad27Δ, or rad27Δ yeast cells with indicated pol3 knock-in mutations grown at 30° C., 25° C., or 37° C. for 48 hour. POL3::HIS3 represents the POL3 (WT or mutant) alleles with a linked HIS3 selection marker gene.

FIGS. 2A-2I. Restrictive temperature stress induces 3′ flap-based OFM and results in alternative duplications. FIG. 2A: Somatic mutation frequencies and types as determined by WGS, in WT and rad27Δ cells grown at 30° C. or 37° C. for 4 h. FIG. 2B: Lengths of inserted DNA sequences in duplications in rad27Δ cells grown at 30° C. or 37° C. for 4 h. FIG. 2C: Top, diagram of classic and alternative duplications. Bottom, frequencies of classic and alternative duplications.

FIG. 2D Predicted structures leading to three types of alternative duplications. FIG. 2E: Schematic for specific labeling of 3′ flaps in genomic DNA. Dots: dideoxyribonucleotide; Star: ³²P-deoxyribonucleotide. FIG. 2F: Levels of 3′ flaps in genomic DNA from WT, rad27Δ, or rad27Δ pol3 ITD (rad27Δ-ITD) cells grown at 30° C. or 37° C. for 4 h. FIG. 2G: Levels of 3′ flaps in genomic DNA from rad27Δ cells grown at 37° C. with or without pre-treatment with Pol₀. FIGS. 2H-2L Reconstitution assays using ³²P-labeled 3′ flap substrate S3 (FIG. 2H) or ³²P-labeled secondary structure-forming 3′ flap substrate S4 or S5 (FIG. 2I). Substrate structures are shown above a representative image of 8% denaturing PAGE. DNA substrates (S3, S4, S5), cleavage products (Cleaved S3, S4), unligated extended 3′ flap intermediates (Extended S4, S5), and ligated extended products (Ligated P3, P4, P5) are indicated. dNTP: deoxyribonucleotides.

FIGS. 3A-3E. Polδ-ITD suppresses 5′ flap formation. (A and B) In vitro assays of primer extension (FIG. 3A) and displacement DNA synthesis (FIG. 3B) by WT Polδ or Polδ-ITD. DNA substrates and primer extension products in panel A, and DNA substrates, gap filling products, or displacement DNA synthesis products in panel B are indicated. (FIG. 3C) Mean Can^{r} mutation rates of WT (n=5), rad27Δ (n=5), or rad27Δ yeast cells with knock-in of pol3 458-477 ITD (n=3), pol3 R470G (n=2), pol3 R475I (n=3), pol3 A484V (n=2), and pol3 S847Y (n=2). Error bars indicate s.d. p values were calculated using student's t test. (FIG. 3D) Can^{r} mutation spectra of the indicated yeast strains. Values shown are percentages of the specific type of Can^{r} mutation in WT (n=22), rad27Δ (n=20), rad27A with knock-in of pol3 458-477 ITD (n=21), pol3 R470G (n=10), pol3 R475I (n=21), pol3 A484V (n=19), or pol3 S847Y (n=21). (FIG. 3E) Mutation frequencies and types present across the genome, as determined by WGS, in WT, rad27Δ, or rad27Δ cells with indicated pol3 knock-in mutations grown at 30° C. (n=1).

FIGS. 4A-4G. Restrictive temperature stress activates signaling that facilitates error-prone OFM and generation of rad27Δ revertants. (FIG. 4A) Up-regulated or down-regulated gene ratios in Dun1-, Rad53-, Mec1-, or Tell-controlled pathways in WT or rad27Δ yeast cells exposed to 30° C. (4 h) or 37° C. (4 h). p values were calculated using the hypergeometric test; n.s., not significant. (FIG. 4B) Top, western blot of chromatin-associated Dun1 protein in WT or rad27Δ cells exposed to 30° C. (4 h) or 37° C. (4 h). Histone H2B was used as a loading control for the chromatin fraction in each sample. dun1Δ is a negative control. Bottom, quantification of chromatin-associated Dun1 relative to the loading control. The Dun1 level in rad27Δ cells grown at 30° C. was arbitrarily set as 1, and the relative Dun1 levels in other samples were calculated by dividing their Dun1 levels by that in rad27Δ cells grown at 30° C. Error bars indicate s.e.m (n=4 biological replicates). (FIG. 4C) Levels of 3′ flaps in genomic DNA from WT, dun1A, rad27Δ, or rad27Δ dun1A double-mutant cells grown at 30° C. or 37° C. for 4 h. (FIG. 4D) Mean Can^{r} mutation rates of WT, rad27Δ, dun1Δ and rad27Δ dun1Δ cells exposed to 30° C. (4 h) or 37° C. (4 h). Error bars indicate s.d. (n=3 independent assays). (FIG. 4E) Median revertant frequencies of rad27Δ or rad27Δ dun1Δ cells (n=3 independent assays). (FIG. 4F) Percentage of rad27Δ or rad27Δ dun1Δ revertants that carry a pol3 mutation (n=19 for each strain). FIG. 4G: Schematic illustrating error-free 5′ flap-mediated OFM in WT cells, error-prone 3′ flap-mediated OFM and the corresponding consequences in rad27Δ cells under restrictive temperature stress (37° C.), and the impact of Polδ-ITD on OFM in the revertant.

FIG. 5. Knock-in of POL3 mutations in rad27Δ cells reverses conditional lethality at 37° C. Viability of WT, rad27Δ, and rad27Δ cells carrying the WT or the POL3 mutant allele (ITD, R470G, R475I, or A484V) was analyzed using spot assays. Yeast cells of the indicated genetic backgrounds were serially diluted, spotted onto YPD plates, and incubated at 30° C., 25° C., or 37° C. for 48 h. rad27Δ::URA3 or rad27Δ::LEU2 represent the rad27Δ allele with a linked URA3 or LEU2 selection marker gene, respectively.

FIG. 6. pol3 ITD-bearing rad27Δ revertant mutations or pol3 ITD knock-in of rad27Δ cells partially reverses MMS-induced lethality. Viability of WT, pol3 ITD knock-in, rad27Δ, rad27Δ with the pol3 ITD knock-in mutation, and a rad27Δ revertant carrying a pol3 ITD mutation was analyzed using spot assays. Yeast cells of the indicated genetic backgrounds were serially diluted and spotted on YPD plates containing the indicated level of MMS. The plates were incubated at 30° C. for 48 hours.

FIGS. 7A-7B. The pol3 ITD mutation that occurred frequently in rad27Δ DNA sequences. (FIG. 7A) DNA and amino acid (a.a.) sequences comprising the duplication unit; DNA and a.a. sequences comprising the spacer between the duplication units. (FIG. 7B) The predicted secondary structure of the pol3 458-477 ITD. The 3′ end of the upstream duplication unit anneals to its complementary sequence at the downstream duplication unit. Extension of the annealed 3′ end produces the spacer DNA sequence (highlighted in gray). This ITD mutation occurred in 19 of the 31 independent revertant colonies.

FIGS. 8A-8D. Representative DNA sequences (FIG. 8A) and predicted hairpin structure (FIGS. 8B-8D) of the three types of alterative duplications detected in rad27Δ cells (37° C., 4 h). DNA sequences comprising the duplication unit; DNA sequences comprising the spacers between the duplication units; in panel A, DNA sequences that are complementary elements that can anneal for extension and formation of repairable nicks/gaps. Predicted hairpin structures of three representative alternative duplications. Panels B, C, and D show the predicted Type 1, Type 2, and Type 3 alternative duplications, respectively.

FIGS. 9A-9B. Reconstitution assays using ³²P-labeled 3′ flap substrate. (FIG. 9A) Schematic of sequential reconstitution of PolO-mediated OFM involving 3′ flap cleavage, gap filling, and DNA ligation. (FIG. 9B) Reconstitution assays (30° C.). Top panels: Diagrams of DNA substrates with a 1 nt gap and a 3′ flap of 0 (51), 10 (S2), or 20 nt (S3) that were used for the assays. Bottom panel: Representative PAGE image of the assay. DNA substrates (51, S2, S3), cleavage products (Cleaved 51, S2, S3), and ligated products (Ligated P1, P2, P3) are indicated.

FIGS. 10A-10D. Reconstituted 3′ flap-based OFM on DNA substrates with long 3′ flaps. (FIG. 10A) Diagrams of DNA substrates with a 1 nt gap and a 3′ flap of 40 nt (S6) or a fold-back forming 3′ flap (S7) that were used for the assays shown in panel B. (FIG. 10B) Representative reconstitution assays (30° C.) for 3′ flap-based OFM on DNA substrates S6 and S7. DNA substrate, cleavage products, unligated, and extended products (unligatable extended S6 or S7) are indicated. Substrates and products were analyzed using 15% denaturing PAGE. (FIG. 10C) Schematic elucidating formation of unligatable extended products in reactions using the 3′ flap substrate S6. Mis-alignment of the 3′ flap with the template via a 4 nt microhomology sequence, which is 15 nt from the 5′ end of the template, prevented its degradation by the 3′ exonuclease activity of Polo. Extension of this structure resulted in a 95 nt unligatable extended product (unligatable extended S6). (FIG. 10D) Formation of fold-back secondary structure in a 3′ flap via the homology sequence prevented its degradation, and extension of this structure resulted in 114 nt unligatable extended product (unligatable extended S7).

FIGS. 11A-11B. 3′ flap processing by nuclear extract (NE) from WT or rad27Δ yeast cells grown at 30° C. or 37° C. The 3′ flap substrate S6 (FIG. 11A) or the fold-back forming 3′ flap substrate S7 (FIG. 11B) was incubated with purified recombinant Polδ (100 ng) or NE (1 jig each reaction) from the indicated yeast cell culture at 37° C. for 15 min. Substrates and products were analyzed using 15% denaturing PAGE.

FIG. 12. Relative gene expression levels of representative 3′ nucleases in WT or rad27Δ cells. Gene expression profiling of WT or rad27Δ cells grown at 30° C. or 37° C. was conducted using RNA-seq. The relative expression levels were calculated by normalizing the fragments per kilobase of transcript per million mapped reads (FPKM) of a specific gene with the FPKM of ACT1 in a sample. Values shown are averages of two biological replicates. Error bars indicate s.d.

FIGS. 13A-13B. Levels of WT Pol3 and Pol3 ITD at 30° C. or 37° C. A DNA fragment encoding a flag tag was knocked into rad27A yeast cell bearing a WT POL3 or pol3 ITD gene, to express Flag-tagged WT Pol3 (Flag-Pol3) or Flag-tagged Pol3 ITD (Flag-ITD). FIG. 13A: Western blot verifying knock-in of the Flag tag-encoding sequence at the WT POL3 or pol3 ITD gene in the rad27Δ strain using anti-Flag antibody. FIG. 13B: Western blot for protein expression of Flag-tagged WT Pol3 (WT) and Pol3 ITD (ITD) in rad27Δ cells at 30° C. or 37° C. using anti-Flag antibody. Ponceau S staining of total proteins was used as a loading control in both panels.

FIGS. 14A-14B. Nuclease activity of WT Polδ and Polδ-ITD. Recombinant WT Polδ or Polδ-ITD protein (20 nM) was incubated with ³²P-labeled DNA substrates (100 nM, top panels) that were identical to the substrates for primer extension (panel A) and displacement DNA synthesis (panel B) assays (FIGS. 29-30). The reactions were carried out at 30° C. for 2.5, 5, 10, 20, 40 min and analyzed using 15% denaturing PAGE.

FIGS. 15A-15B. 3′ flap processing and 3′ flap-based OFM by recombinant WT Polδ, nuclease-dead Polδ D520E, and Polδ-ITD. FIG. 15A: 3′ flap cleavage and subsequent 3′ exonuclease cleavage of ³²P-labeled 3′ flap DNA substrate by WT Polo, nuclease-dead Polo D520E, and Polo-ITD. Top, diagram of ³²P-labeled substrate with a 40 nt 3′ flap (S6) (FIGS. 29-30). Middle, representative PAGE image of the nuclease activity assay. The substrate and cleavage products are indicated. Bottom, quantification of the 3′ flap nuclease and subsequent exonuclease cleavage products. FIG. 15B: Reconstituted 3′ flap-based OFM by WT Polδ, nuclease-dead Polδ D520E, and Polδ-ITD. Top, diagram of ³²P-labeled substrate S6. Bottom, representative PAGE image of the nuclease activity assay. The substrate (S6), cleavage product (cleaved S6), unligated extended product (unligated extended S6), and ligated product (P6) are indicated.

FIG. 16. No significant differences in the mutation rate of the WT yeast strain or mutant yeast strains carrying POL3 knock-in mutations. Three representative POL3 mutations (pol3 ITD, R470G, or S847Y) were introduced into the POL3 allele. Mutation rates of the WT and knock-in mutant yeast strains were determined using Can^{r} mutation assay. Values shown are mean±s.d. of three independent assays.

FIG. 17. Diagram of fold-changes in gene expression levels of DNA damage response and repair genes that are mediated by the Mec1-Rad53-Dun1 axis. Gene expression profiling of WT or rad27Δ cells grown at 30° C. or 37° C. was determined using RNA-seq. Fold-changes were calculated by comparing the normalized fragments per kilobase of transcript per million mapped reads (FPKM) of a specific gene in a sample with the normalized FPKM of the same gene in the control (WT, 30° C.), which was arbitrarily set as 1.

FIG. 18. DUN1 knockout has little effect on the viability of rad27Δ cells. Viability of WT, rad27Δ, dun1Δ, or rad27Δ dun1Δ cells was analyzed using spot assays. Yeast cells of the indicated genetic backgrounds were serially diluted, spotted on YPD plates, and incubated at 30° C. or 37° C. for 48 hours.

FIG. 19. Chk1 inhibition blocks lung cancer development in FEN1 mutant mice. WT (n=23) and WT/F343AF344A (WT/FFAA) (n=38) mice were treated with the Chk1 inhibitor SB218078 (0.025 mg/kg body weight). In the untreated control groups, WT (n=31) and WT/FFAA (n=35) were administered DMSO. p value was calculated by the Fisher Exact test.

FIGS. 20A-20C. Impact of deoxyribonucleotides (dNTP) on 3′ flap-based OFM. Reconstituted 3′ flap-based OFM by WT Polδ in the presence of varying concentrations of dNTP (10 μM, 100 μM, and 1 mM) using (FIG. 20A)³²P-labeled 3′ flap substrate S3 and ³²P-labeled secondary structure-forming 3′ flap substrate (FIG. 20B) S4 or (FIG. 20C) S5. Reactions were carried out at 37° C. and analyzed using 8% denaturing PAGE. The substrates (S3, S4, S5), cleavage products (Cleaved S3, S4), extended intermediates (Extended S3, S4, S5), and ligated product (Ligated P3, P4, P5) are indicted.

FIGS. 21A-21B. Nuclease activity of Polδ on a gapped duplex with or without a 1 nt 3′ flap. FIG. 21A: Gapped DNA duplex substrate with or without a 1 nt 3′ flap (FIG. 29). FIG. 21B: WT Polδ (5 nM) was incubated with ³²P-labeled DNA substrates (100 nM). The reactions were carried out at 30° C. for 20 minutes and analyzed using 15% denaturing PAGE.

FIG. 22. SML1 deletion has little effect on the mutation rate of the WT yeast strain or the rad27Δ yeast strain. The mutation rates of WT and rad27Δ. cells at 30° C. or 37° C. were determined using a Can^{r} mutation assay. Values shown are means±s.d. of three independent assays. p value was calculated using Student's t test. n.s., not significant.

FIGS. 23A-23D. 3′ flap OFM-related alternative duplications in human B cell acute lymphoblastic leukemia (ALL) and mouse lung tumors. FIGS. 23A-23C: Germline and somatic duplications in three tumor specimens from randomly selected B cell ALL from the published datasets (41). The duplications present in both tumor and normal samples were considered germline, and those that were present in the tumor samples were considered somatic. FIG. 23A: Frequency of germline and somatic classic and alternative duplications in the three ALL cancer specimens. Classic duplications were simple duplication mutations with no spacer and the alternative duplications were duplications with a spacer DNA sequence between the duplication units (See FIG. 2C for illustration). FIG. 23B: The lengths of spacers in the alternative duplications in the three ALL specimens are shown. They ranged from 1 nt to 70 nt, which was similar to the spacer lengths for stress temperature-induced alternative duplications in rad27.4. FIG. 23C: Relative locations of donor DNA sequences that serve as the template for the intervening spacer DNA. The spacer DNA sequences were mapped across the human genome. For alternative duplications whose duplication unit and the donor DNA sequences for the spacer DNA were on the same chromosome, the linear distance between the duplication unit (break point) and the corresponding spacer donor sequence was calculated. The dashed-boxed portion shows those 3′ flap OFM-related alternative duplications. FIG. 23D: Somatic 3′ flap OFM-related alternative duplications in WT and FEN1 A159V/WT mutant mice. Somatic 3′ flap OFM-related alternative duplications in WT or A159V/WT normal lung or lung tumor (n=1 for each sample) were identified. 3′ flap OFM-related alternative duplications that were only present in the normal lung or lung tumor samples were considered somatic.

FIGS. 24A-24C. SDS-PAGE of purified recombinant Polδ, DNA Lig I, and PCNA proteins. FIG. 24A: Yeast WT Polδ and Polδ-ITD. Three subunits, Pol3 (WT or ITD), Pol31, and Pol32, of yeast Polδ were co-expressed in yeast and purified using chromatography as described in the Methods section. The purity of WT Polδ or Polδ-ITD was evaluated by resolving proteins on 10% SDS-PAGE. FIG. 24B: 6His-tagged DNA Lig I was expressed in E. coli and purified using chromatography as described in the Methods section. The purity of DNA Lig I was evaluated by resolving proteins using 10% SDS-PAGE. S1: the eluted peak fraction of Ni-NTA chromatography, S2: the eluted peak fraction of heparin chromatography. FIG. 24C: 6His-tagged DNA PCNA was expressed in E. coli and purified by chromatography as described in the Methods section. The purity of PCNA was evaluated by resolving the protein using 15% SDS-PAGE. All gels were stained with Coomassie blue 250. M: protein markers.

FIG. 25 provides a summary of enriched mutations in one rad27Δ reverant.

FIG. 26 provides a viability test of rad27Δ pol3 ITD exo1Δ cells via genetic crosses and random spore analysis.

FIG. 27 provides a viability test of rad27Δ pol3 ITD dna2-1 cells via genetic crosses and DNA sequencing.

FIG. 28 shows the yeast strains for the genetic studies described herein.

FIG. 29 shows the labeled DNA substrates used for primer extension or strand displacement DNA synthesis in the examples described herein.

FIG. 30 shows the synthetic oligonucleotides developed and prepared in the examples described herein.

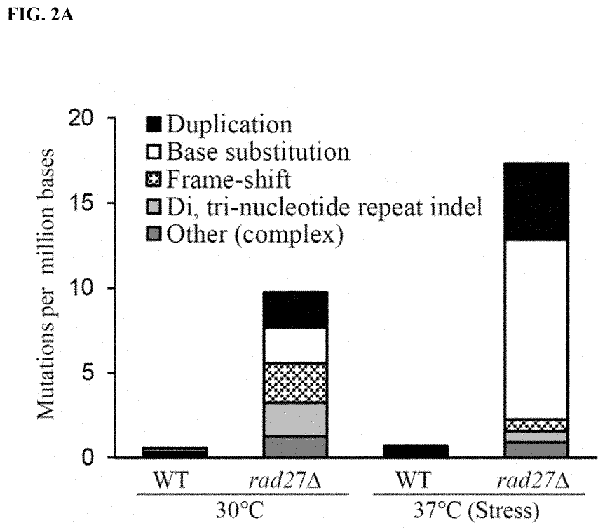

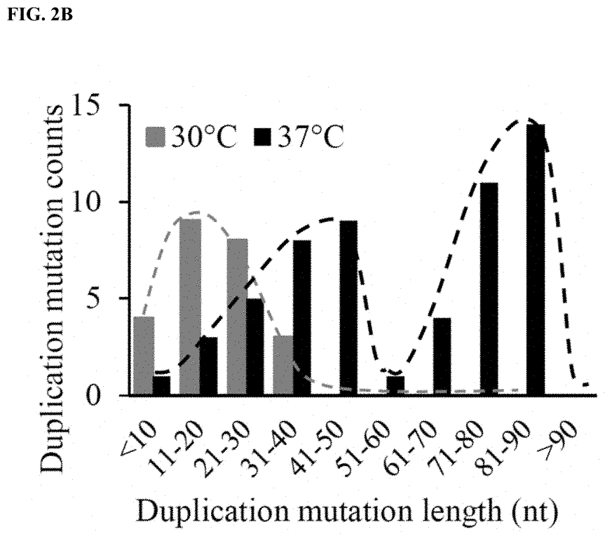

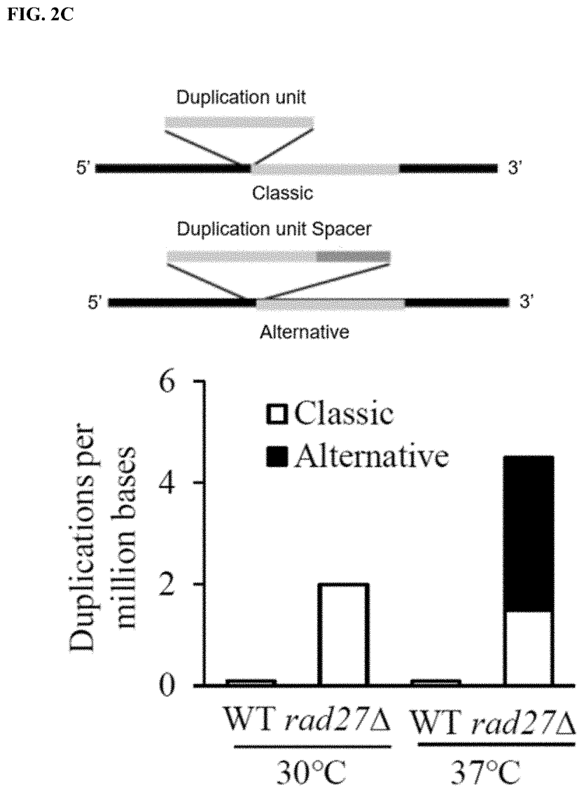

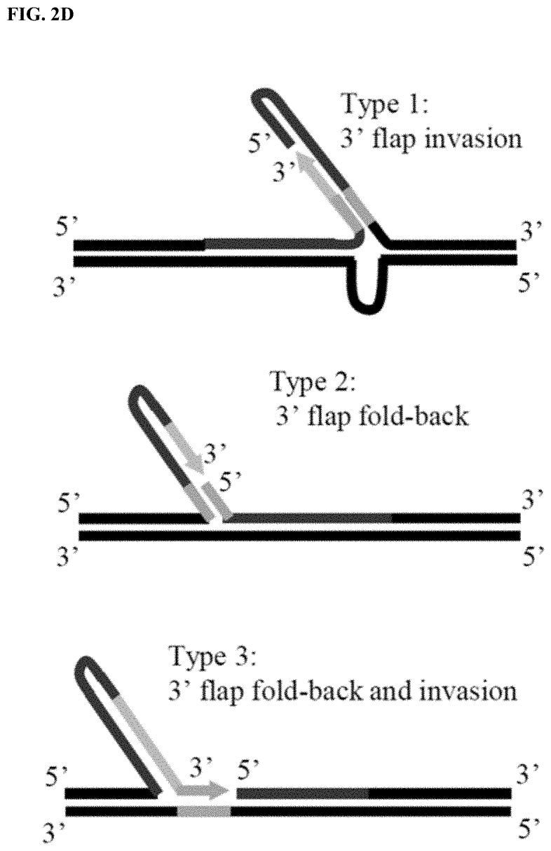

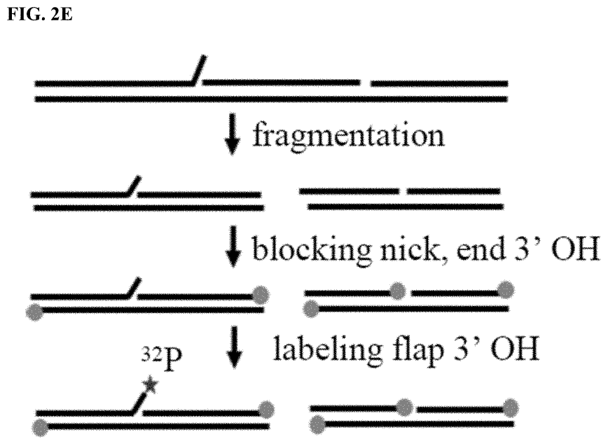

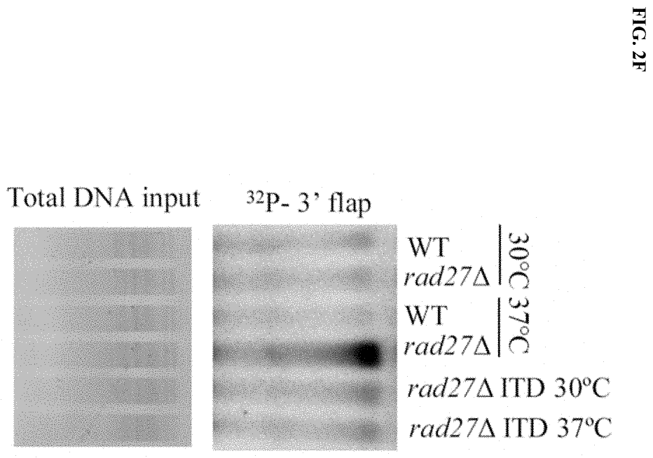

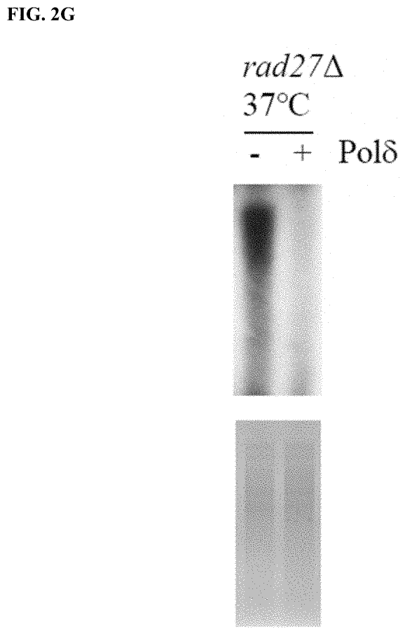

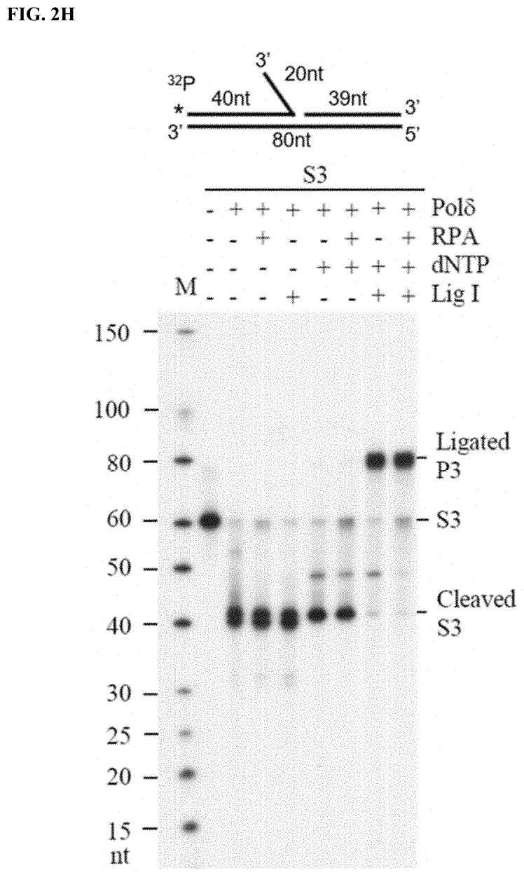

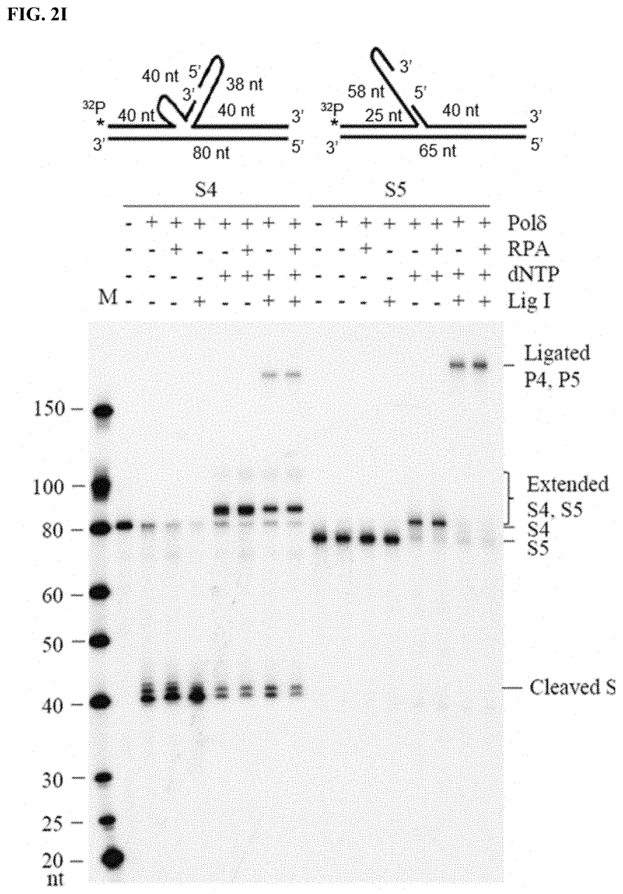

The drawings described herein are also available at Sun et al, Science, 374(6572):1252-1258 (2021). Color versions of FIGS. 2C-2E, 4G, 7A, 8A, 8B, 10C, 10D, 23A-23C, 24A-24C are set forth in Sun et al, Science, 374(6572):1252-1258 (2021) and are incorporated by reference herein in their entirety and for all purposes.

### DETAILED DESCRIPTION

### Definitions

Unless defined otherwise, technical and scientific terms used herein have the same meaning as commonly understood by a person of ordinary skill in the art. See, e.g., Singleton et al., Dictionary of Microbiology and Molecular Biology, 2nd ed., J. Wiley & Sons (New York, N.Y. 1994); Sambrook et al., Molecular Cloning, A Laboratory Manual, Cold Springs Harbor Press (Cold Springs Harbor, NY 1989). Any methods, devices and materials similar or equivalent to those described herein can be used in the practice of this disclosure. The following definitions are provided to facilitate understanding of certain terms used frequently herein and are not meant to limit the scope of the present disclosure.

An “Okazaki fragment” is a short sequence of DNA nucleotides (approximately 100 to 300 base pairs long in eukaryotes) which are synthesized discontinuously and later linked together by the enzyme DNA ligase to create the lagging strand during DNA replication.

“3′ flap” and “3′ flap Okazaki fragment” are terms known in the art and are used as such herein. In embodiments, a 3′ flap Okazaki fragment is a single stranded nucleic acid sequence that forms part of a genomic DNA resulting from transformation of an un-processed 5′ flap Okazaki fragment (as described herein). In embodiments, the 3′ flap Okazaki fragment is removed by a 3′ flap endonuclease. In embodiments, the 3′ flap Okazaki fragment forms a simple 3′ flap (as described herein). In embodiments, the 3′ flap Okazaki fragment forms a secondary structure. In embodiments, the secondary structure facilitates 3′ end extension rather than degradation, thereby producing an alternative duplication (e.g. with short spacer sequences). In embodiments, the alternative duplication comprises from 1 to 100 nucleotides. In embodiments, the alternative duplication comprises from 1 to 90 nucleotides. In embodiments, the alternative duplication comprises from 1 to 80 nucleotides. In embodiments, the alternative duplication comprises from 1 to 70 nucleotides. In embodiments, the alternative duplication is a 3′ flap invasion, a 3′ flap fold-back, or a 3′ flap fold-back and invasion. In embodiments, the alternative duplication is a 3′ flap invasion. In embodiments, the alternative duplication is a 3′ flap fold-back. In embodiments, the alternative duplication is a 3′ flap fold-back and invasion.

Methods for detecting 3′ flap Okazaki fragments are known in the art. For example, genomic DNA is extracted from cells (such as cancer cells) and purified. The purified genomic DNA is incubated with restriction enzymes. The 3′ OH ends at DNA nicks, DNA gaps, and DNA ends are blocked and the free 3′ OH ends at the 3′ flap are labeled and detected. A detailed procedure is set forth in Example 2.

“An elevated level of a 3′ flap Okazaki fragment” as referred to herein is an elevated level of 3′ flap Okazaki fragments compared to a control.

A “DNA Damage Response Inhibitor” refers to any compound (e.g., small molecule, biologic) that decreases or prevents formation of 3′ flap Okazaki fragments relative to the activity or function of Okazaki fragment maturation in the absence of the DNA Damage Response Inhibitor. In embodiments, the DNA Damage Response Inhibitor slows, prevents, or reverses the progression of cancer by increasing Okazaki fragment maturation, inhibiting (partially or completely) impairment of Okazaki fragment maturation, or preventing impairment of Okazaki fragment maturation (e.g., decreasing or preventing formation of 3′ flap Okazaki fragments).

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

The terms “DNA” or “deoxyribonucleic acid” refer to a molecule composed of two polynucleotide chains that coil around each other to form a double helix carrying genetic instructions for the development, functioning, growth and reproduction of all known organisms and many viruses. DNA and ribonucleic acid (RNA) are nucleic acids. Alongside proteins, lipids and complex carbohydrates (polysaccharides), nucleic acids are one of the four major types of macromolecules that are essential for all known forms of life. The two DNA strands are known as polynucleotides as they are composed of simpler monomeric units called nucleotides. Each nucleotide is composed of one of four nitrogen-containing nucleobases (cytosine (C), guanine (G), adenine (A) or thymine (T)), a sugar called deoxyribose, and a phosphate group. The nucleotides are joined to one another in a chain by covalent bonds (known as the phosphodiester linkage) between the sugar of one nucleotide and the phosphate of the next, resulting in an alternating sugar-phosphate backbone. The nitrogenous bases of the two separate polynucleotide strands are bound together, according to base pairing rules (A with T and C with G), with hydrogen bonds to make double-stranded DNA. The complementary nitrogenous bases are divided into two groups, pyrimidines and purines. In DNA, the pyrimidines are thymine and cytosine; the purines are adenine and guanine.

The term “DNA fraction” refers to DNA or portion of DNA partitioned from other molecules of a biological sample (e.g., biological fluid, such as blood, plasma, or serum).

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another: (1) alanine (A), glycine (G); (2) aspartic acid (D), glutamic acid (E); (3) asparagine (N), glutamine (Q); (4) arginine (R), lysine (K); (5) isoleucine (I), leucine (L), methionine (M), valine (V); (6) phenylalanine (F), tyrosine (Y), tryptophan (W); (7) serine (S), threonine (T); and (8) cysteine (C), methionine (M).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 95%, 98%, or 99% identity over a specified region, e.g., of the entire polypeptide sequences or individual domains of the polypeptide sequence), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 50 nucleotides in length, or preferably over a region that is 100 to 500 or 1000 or more nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, less than about 0.01, or less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “isolated,” when applied to a nucleic acid or protein, denotes that the nucleic acid or protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A nucleic acid or protein that is the predominant species present in a preparation is substantially purified. The term “purified” denotes that a nucleic acid or protein gives rise to essentially one band in an electrophoretic gel. Particularly, it means that the protein is at least 85% pure, at least 90% pure, at least 95% pure, or at least 99% pure.

As used herein, the term “cancer” refers to all types of cancer, neoplasm or malignant tumors found in mammals (e.g., humans), including leukemia, lymphoma, carcinomas and sarcomas. Exemplary cancers that may be treated with a compound or method provided herein include cancer of the thyroid, endocrine system, brain, breast, cervix, colon, head and neck, liver, kidney, lung, non-small cell lung, melanoma, mesothelioma, ovary, sarcoma, stomach, uterus medulloblastoma, colorectal cancer, or pancreatic cancer. Additional examples include Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, lymphomas, thyroid cancer, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

Cancer model organism, as used herein, is an organism exhibiting a phenotype indicative of cancer, or the activity of cancer causing elements, within the organism. The term cancer is defined above. A wide variety of organisms may serve as cancer model organisms, and include for example, cancer cells and mammalian organisms such as rodents (e.g. mouse or rat) and primates (such as humans). Cancer cell lines are widely understood by those skilled in the art as cells exhibiting phenotypes or genotypes similar to in vivo cancers. Cancer cell lines as used herein includes cell lines from animals (e.g. mice) and from humans.

An “anticancer agent” as used herein refers to a molecule (e.g. compound, peptide, protein, nucleic acid, 0103) used to treat cancer through destruction or inhibition of cancer cells or tissues. Anticancer agents may be selective for certain cancers or certain tissues. In embodiments, anticancer agents herein may include epigenetic inhibitors and multi-kinase inhibitors.

“Anti-cancer agent” and “anticancer agent” are used in accordance with their plain ordinary meaning and refers to a composition (e.g. compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethylmelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin (including recombinant interleukin II, or rlL.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol™ (i.e. paclitaxel), Taxotere™, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), LS-4559-P (Pharmacia, i.e. LS-4577), LS-4578 (Pharmacia, i.e. LS-477-P), LS-4477 (Pharmacia), LS-4559 (Pharmacia), RPR-112378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e. ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e. LY-355703), AC-7739 (Ajinomoto, i.e. AVE-8063A and CS-39.HCl), AC-7700 (Ajinomoto, i.e. AVE-8062, AVE-8062A, CS-39-L-Ser.HCl, and RPR-258062A), Vitilevuamide, Tubulysin A, Canadensol, Centaureidin (i.e. NSC-106969), T-138067 (Tularik, i.e. T-67, TL-138067 and TI-138067), COBRA-1 (Parker Hughes Institute, i.e. DDE-261 and WHI-261), H10 (Kansas State University), H16 (Kansas State University), Oncocidin A1 (i.e. BTO-956 and DIME), DDE-313 (Parker Hughes Institute), Fijianolide B, Laulimalide, SPA-2 (Parker Hughes Institute), SPA-1 (Parker Hughes Institute, i.e. SPIKET-P), 3-IAABU (Cytoskeleton/Mt. Sinai School of Medicine, i.e. MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e. MF-191), TMPN (Arizona State University), Vanadocene acetylacetonate, T-138026 (Tularik), Monsatrol, lnanocine (i.e. NSC-698666), 3-IAABE (Cytoskeleton/Mt. Sinai School of Medicine), A-204197 (Abbott), T-607 (Tuiarik, i.e. T-900607), RPR-115781 (Aventis), Eleutherobins (such as Desmethyleleutherobin, Desaetyleleutherobin, lsoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e. NSCL-96F037), D-68838 (Asta Medica), D-68836 (Asta Medica), Myoseverin B, D-43411 (Zentaris, i.e. D-81862), A-289099 (Abbott), A-318315 (Abbott), HTI-286 (i.e. SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), immunotherapy (e.g., cellular immunotherapy, antibody therapy, cytokine therapy, combination immunotherapy, etc.), radioimmunotherapy (e.g., anti-CD20 monoclonal antibody conjugated to ¹¹¹In, ⁹⁰Y, or ¹³¹I, etc.), immune checkpoint inhibitors (e.g., CTLA4 blockade, PD-1 inhibitors, PD-L1 inhibitors, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™), panitumumab (Vectibix™), vandetanib (Caprelsa™) afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like.

An “inhibitor” refers to a compound (e.g. compounds described herein) that reduces activity when compared to a control, such as absence of the compound or a compound with known inactivity.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

The term “activation,” “activate,” “activating,” “activator” and the like in reference to a protein-inhibitor interaction means positively affecting (e.g. increasing) the activity or function of the protein relative to the activity or function of the protein in the absence of the activator. In embodiments activation means positively affecting (e.g. increasing) the concentration or levels of the protein relative to the concentration or level of the protein in the absence of the activator. The terms may reference activation, or activating, sensitizing, or up-regulating signal transduction or enzymatic activity or the amount of a protein decreased in a disease. Thus, activation may include, at least in part, partially or totally increasing stimulation, increasing or enabling activation, or activating, sensitizing, or up-regulating signal transduction or enzymatic activity or the amount of a protein associated with a disease (e.g., a protein which is decreased in a disease relative to a non-diseased control). Activation may include, at least in part, partially or totally increasing stimulation, increasing or enabling activation, or activating, sensitizing, or up-regulating signal transduction or enzymatic activity or the amount of a protein

The terms “agonist,” “activator,” “upregulator,” etc. refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist.

The term “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. In embodiments inhibition means negatively affecting (e.g. decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

The terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components.

The term “administering” means oral administration, administration as a suppository, topical contact, intravenous, parenteral, intraperitoneal, intramuscular, intralesional, intrathecal, intranasal, or subcutaneous administration to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

The terms “treating” or “treatment” refers to any indicia of success in the therapy or amelioration of a disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is no prophylactic treatment.

The term “prevent” refers to a decrease in the occurrence of disease symptoms in a patient. As indicated above, the prevention may be complete (no detectable symptoms) or partial, such that fewer symptoms are observed than would likely occur absent treatment.

“Patient” or “subject in need thereof” refers to a living organism suffering from or prone to a disease or condition that can be treated by administration of a pharmaceutical composition as provided herein. Non-limiting examples include humans, other mammals, bovines, dogs, monkeys, and other non-mammalian animals. In some embodiments, a patient is human.

“Pharmaceutically acceptable excipient” refers to a substance that aids the administration of an active agent to and absorption by a subject and can be included in the compositions of the disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such compositions can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the antibodies of the disclosure. One of skill in the art will recognize that other pharmaceutical excipients are useful. The term “excipient” can be used interchangeably with other terms of art, such as “carrier” or “diluent.”

Solutions of the antibodies can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these compositions can contain a preservative to prevent the growth of microorganisms.

For parenteral administration in an aqueous solution, for example, the solution should be suitably buffered and the liquid diluent first rendered isotonic with sufficient saline or glucose. Aqueous solutions, in particular, sterile aqueous media, are especially suitable for intravenous, intramuscular, subcutaneous and intraperitoneal administration. For example, one dosage could be dissolved in 1 ml of isotonic NaCl solution and either added to 1000 ml of hypodermoclysis fluid or injected at the proposed site of infusion.

Sterile injectable solutions can be prepared by incorporating the antibodies in the required amount in the appropriate solvent followed by filtered sterilization. Generally, dispersions are prepared by incorporating the antibodies into a sterile vehicle which contains the basic dispersion medium. Vacuum-drying and freeze-drying techniques, which yield a powder of the active ingredient plus any additional desired ingredients, can be used to prepare sterile powders for reconstitution of sterile injectable solutions. The composition of more, or highly, concentrated solutions for direct injection is also contemplated. Solvents, such as dimethyl sulfoxide, can be used for extremely rapid penetration, delivering high concentrations of the active agents to a small area.

Pharmaceutical compositions can be delivered via intranasal or inhalable solutions or sprays, aerosols or inhalants. Nasal solutions can be aqueous solutions designed to be administered to the nasal passages in drops or sprays. Nasal solutions can be prepared so that they are similar in many respects to nasal secretions. Thus, the aqueous nasal solutions usually are isotonic and slightly buffered to maintain a pH of 5.5 to 6.5. In addition, antimicrobial preservatives, similar to those used in ophthalmic compositions and appropriate drug stabilizers, if required, may be included in the formulation. Various commercial nasal compositions are known.

Oral formulations can include excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In aspects, oral pharmaceutical compositions will comprise an inert diluent or edible carrier, or they may be enclosed in hard or soft shell gelatin capsule, or they may be compressed into tablets, or they may be incorporated directly with the food. For oral therapeutic administration, the antibodies may be incorporated with excipients and used in the form of ingestible tablets, buccal tablets, troches, capsules, elixirs, suspensions, syrups, wafers, and the like. The percentage of the compositions may, of course, be varied and may conveniently be between about 1 to about 75% of the weight of the unit. The amount of antibodies in such compositions is such that a suitable dosage can be obtained.

The formulations of antibodies can be presented in unit-dose or multi-dose sealed containers, such as ampules and vials. Thus, the composition can be in unit dosage form. In such form the composition is subdivided into unit doses containing appropriate quantities of antibodies. Thus, the compositions can be administered in a variety of unit dosage forms depending upon the method of administration.

Pharmaceutical compositions include compositions wherein the active ingredient is contained in a therapeutically effective amount, i.e., in an amount effective to achieve its intended purpose. The actual amount effective for a particular application will depend, inter alia, on the condition being treated, as judged by a practitioner in the medical arts.

A “effective amount” is an amount sufficient to accomplish a stated purpose relative to the absence of the compound (e.g. achieve the effect for which it is administered, treat a disease, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a compound is an amount of that compound, when administered to a subject, will have the intended prophylactic effect, e.g., preventing or delaying the onset (or reoccurrence) of a disease, pathology or condition, or reducing the likelihood of the onset (or reoccurrence) of a disease, pathology, or condition, or their symptoms. The full prophylactic effect does not necessarily occur by administration of one dose, and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of compounds that are capable of achieving the methods described herein, as measured using the methods described herein or known in the art. As is well known in the art, therapeutically effective amounts for use in humans can also be determined from animal models. For example, a dose for humans can be formulated to achieve a concentration that has been found to be effective in animals. The dosage in humans can be adjusted by monitoring effectiveness and adjusting the dosage upwards or downwards, as described above. Adjusting the dose to achieve maximal efficacy in humans based on the methods described above and other methods is well within the capabilities of the ordinarily skilled artisan.

Dosages and frequency (single or multiple doses) of the compounds may be varied depending upon the requirements of the patient. The dose administered to a patient should be sufficient to affect a beneficial therapeutic response in the patient over time. The size of the dose also will be determined by the existence, nature, and extent of any adverse side-effects. Determination of the proper dosage for a particular situation is within the skill of the art. Generally, treatment is initiated with smaller dosages which are less than the optimum dose of the DNA

Damage Response Inhibitor and/or anticancer agent. Thereafter, the dosage is increased by small increments until the optimum effect under circumstances is reached. Dosage amounts and intervals can be adjusted individually to provide levels of the DNA Damage Response Inhibitor and/or anticancer agent effective for the particular clinical indication being treated. This will provide a therapeutic regimen that is commensurate with the severity of the individual's disease state. In embodiments, the DNA Damage Response Inhibitor and/or anticancer agent is administered at an amount from about 0.001 μg to about 10,000 g.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of monoclonal antibodies by considering factors such as compound potency, relative bioavailability, patient body weight, presence and severity of adverse side effects.

The term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

“Biological sample” refers to any biological sample taken from a subject. Biological samples include blood, plasma, serum, tumors, tissue, cells, and the like. In embodiments, the biological sample is a cell. In embodiments, the biological sample is cancer cell. In embodiments, the biological sample is genomic DNA in a cancer cell. In embodiments, the biological sample is genomic DNA in a cell. Biological samples can be taken from a subject by methods known in the art, and can be analyzed by methods known in the art.

A “control” sample or value refers to a sample that serves as a reference, usually a known reference, for comparison to a test sample. For example, a test sample can be taken from a patient suspected of having a given disease (cancer) and compared to samples from a known cancer patient, or a known normal (non-diseased) individual. A control can also represent an average value gathered from a population of similar individuals, e.g., cancer patients or healthy individuals with a similar medical background, same age, weight, etc. A control value can also be obtained from the same individual, e.g., from an earlier-obtained sample, prior to disease, or prior to treatment. One of skill will recognize that controls can be designed for assessment of any number of parameters. In embodiments, a control is a negative control. In embodiments, such as some embodiments relating to detecting the presence of 3′ flap Okazaki fragments, a control comprises the average amount of expression (e.g., protein or nucleic acid) of infiltration (e.g., number or percentage of cells in a population of cells) in a population of subjects (e.g., with cancer) or in a healthy or general population. In embodiments, the control comprises an average amount (e.g. percentage or number of infiltrating cells or amount of expression) in a population in which the number of subjects (n) is 5 or more, 10 or more, 25 of more, 50 or more, 100 or more, 1000 or more, 5000 or more, or 10000 or more. In embodiments, the control is a standard control. One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

Methods

The disclosure provides a method for detecting a 3′ flap Okazaki fragment in a patient having cancer, the method comprising detecting a 3′ flap Okazaki fragment in a biological sample obtained from the patient having cancer. In embodiments, the biological sample contains an elevated level of the 3′ flap Okazaki fragment relative to a control. In embodiments, the biological sample is a cancer cell. In embodiments, the biological sample is genomic DNA in a cancer cell. In embodiments, the cancer is leukemia or lung cancer. In embodiments, the cancer is leukemia. In embodiments, the cancer is acute lymphoblastic leukemia. In embodiments, the cancer is lung cancer. In embodiments, the cancer is EGFR-mutated lung cancer. In embodiments, the cancer is small cell lung cancer. In embodiments, the cancer is EGFR-mutated small cell lung cancer. In embodiments, the cancer is non-small cell lung cancer. In embodiments, the cancer is EGFR-mutated non-small cell lung cancer. In embodiments, the DNA Damage Response Inhibitor is an ATM kinase inhibitor, an ATR kinase inhibitor, a Chk1 kinase inhibitor, a Chk2 kinase inhibitor, or a combination of two or more thereof. In embodiments, the DNA Damage Response Inhibitor is an ATM kinase inhibitor. In embodiments, the ATM kinase inhibitor is AZD0156, KU-60019, or AZD1390. In embodiments, the DNA Damage Response Inhibitor is an ATR kinase inhibitor. In embodiments, the ATR kinase inhibitor is berzosertib (also known as M6620 or VX-970) or elimusertib (also known as Bay895344). In embodiments, the DNA Damage Response Inhibitor is a Chk1 kinase inhibitor. In embodiments, the Chk1 kinase inhibitor is SRA737 or prexasertib (also known as Ly2606368). In embodiments, the DNA Damage Response Inhibitor is a Chk2 kinase inhibitor. In embodiments, the Chk2 kinase inhibitor is Ly2606368. In embodiments, the methods further comprise administering to the patient a therapeutically effective amount of an anticancer agent. In embodiments, the anticancer agent is a cytotoxic agent. In embodiments, the cytotoxic agent is an alkylating agent, an antimetabolite, a mitotic inhibitor, a topoisomerase inhibitor, or a combination of two or more thereof. In embodiments, the cytotoxic agent is an alkylating agent. In embodiments, the alkylating agent is bendamustine, busulfan, carboplatin, carmustine, chlorambucil, cisplatin, cyclophosphamide, decarbazine, estramustine, hydroxyurea, ifosfamide, lomustine, mechlorethamine, melphalan, oxaliplatin, procarbazine, streptozocin, temozolomide, thiotepa, treosulfan, or a combination of two or more thereof. In embodiments, the cytotoxic agent is an antimetabolite. In embodiments, the antimetbolite is azacitidine, capecitabine, cladribine, cytarabine, fludarabine, fluorouracil, gemcitabine, mercaptopurine, methotrexate, pralatrexate, pemetrexed, pentostatin, raltitrexed, thioguanie, trifluridine/tipiracil, or a combination of two or more thereof. In embodiments, the cytotoxic agent is a mitotic inhibitor. In embodiments, the mitotic inhibitor is cabazitaxel, docetaxel, eribulin, ixabepilone, paclitaxel, vinblastine, vincristine, vinorelbine, or a combination of two or more thereof. In embodiments, the cytotoxic agent a topoisomerase inhibitor. In embodiments, the toposiomerase inhibitor is irinotecan, topotecan, amsacrine, daunorubicin, doxorubicin, epirubicin, idarubicin, etoposide, mitoxantrone, teniposide, or a combination of two or more thereof. In embodiments, the cytotoxic agent is aresenic trioxide, asparaginase, bleomycin, belinostat, dactinomycin, iniparib, lurbinectedin, mitomycin, mitotane, porfimer, romidepsin, vorinostat, or a combination of two or more thereof. In embodiments, the methods further comprising administering to the patient a therapeutically effective amount of radiation therapy. In embodiments, the method of detecting a 3′ flap Okazaki fragment comprises detecting an alternative duplication formed by the 3′ flap Okazaki fragment.

The disclosure provides method for treating cancer in a patient in need thereof comprising administering to the patient a therapeutically effective amount of a DNA Damage Response Inhibitor; wherein a biological sample obtained from the patient contains a 3′ flap Okazaki fragment. In embodiments, the biological sample contains an elevated level of the 3′ flap Okazaki fragment relative to a control. In embodiments, the biological sample is a cancer cell. In embodiments, the biological sample is genomic DNA in a cancer cell. In embodiments, the cancer is leukemia. In embodiments, the cancer is acute lymphoblastic leukemia. In embodiments, the cancer is lung cancer. In embodiments, the cancer is EGFR-mutated lung cancer. In embodiments, the cancer is small cell lung cancer. In embodiments, the cancer is EGFR-mutated small cell lung cancer. In embodiments, the cancer is non-small cell lung cancer. In embodiments, the cancer is EGFR-mutated non-small cell lung cancer. In embodiments, the DNA Damage Response Inhibitor is an ATM kinase inhibitor, an ATR kinase inhibitor, a Chk1 kinase inhibitor, a Chk2 kinase inhibitor, or a combination of two or more thereof. In embodiments, the DNA Damage Response Inhibitor is an ATM kinase inhibitor. In embodiments, the ATM kinase inhibitor is AZD0156, KU-60019, or AZD1390. In embodiments, the DNA Damage Response Inhibitor is an ATR kinase inhibitor. In embodiments, the ATR kinase inhibitor is berzosertib (also known as M6620 or VX-970) or elimusertib (also known as Bay895344). In embodiments, the DNA Damage Response Inhibitor is a Chk1 kinase inhibitor. In embodiments, the Chk1 kinase inhibitor is SRA737 or prexasertib (also known as Ly2606368). In embodiments, the DNA Damage Response Inhibitor is a Chk2 kinase inhibitor. In embodiments, the Chk2 kinase inhibitor is Ly2606368. In embodiments, the methods further comprise administering to the patient a therapeutically effective amount of an anticancer agent. In embodiments, the anticancer agent is a cytotoxic agent. In embodiments, the cytotoxic agent is an alkylating agent, an antimetabolite, a mitotic inhibitor, a topoisomerase inhibitor, or a combination of two or more thereof. In embodiments, the cytotoxic agent is an alkylating agent. In embodiments, the alkylating agent is bendamustine, busulfan, carboplatin, carmustine, chlorambucil, cisplatin, cyclophosphamide, decarbazine, estramustine, hydroxyurea, ifosfamide, lomustine, mechlorethamine, melphalan, oxaliplatin, procarbazine, streptozocin, temozolomide, thiotepa, treosulfan, or a combination of two or more thereof. In embodiments, the cytotoxic agent is an antimetabolite. In embodiments, the antimetbolite is azacitidine, capecitabine, cladribine, cytarabine, fludarabine, fluorouracil, gemcitabine, mercaptopurine, methotrexate, pralatrexate, pemetrexed, pentostatin, raltitrexed, thioguanie, trifluridine/tipiracil, or a combination of two or more thereof. In embodiments, the cytotoxic agent is a mitotic inhibitor. In embodiments, the mitotic inhibitor is cabazitaxel, docetaxel, eribulin, ixabepilone, paclitaxel, vinblastine, vincristine, vinorelbine, or a combination of two or more thereof. In embodiments, the cytotoxic agent a topoisomerase inhibitor. In embodiments, the toposiomerase inhibitor is irinotecan, topotecan, amsacrine, daunorubicin, doxorubicin, epirubicin, idarubicin, etoposide, mitoxantrone, teniposide, or a combination of two or more thereof. In embodiments, the cytotoxic agent is aresenic trioxide, asparaginase, bleomycin, belinostat, dactinomycin, iniparib, lurbinectedin, mitomycin, mitotane, porfimer, romidepsin, vorinostat, or a combination of two or more thereof. In embodiments, the methods further comprising administering to the patient a therapeutically effective amount of radiation therapy.

The disclosure provides method for treating cancer in a patient in need thereof comprising administering to the patient a therapeutically effective amount of a DNA Damage Response Inhibitor and a therapeutically effective amount of a cytotoxic agent; wherein a biological sample obtained from the patient contains a 3′ flap Okazaki fragment. In embodiments, the biological sample contains an elevated level of the 3′ flap Okazaki fragment relative to a control. In embodiments, the biological sample is a cancer cell. In embodiments, the biological sample is genomic DNA in a cancer cell. In embodiments, the cancer is leukemia. In embodiments, the cancer is acute lymphoblastic leukemia. In embodiments, the cancer is lung cancer. In embodiments, the cancer is EGFR-mutated lung cancer. In embodiments, the cancer is small cell lung cancer. In embodiments, the cancer is EGFR-mutated small cell lung cancer. In embodiments, the cancer is non-small cell lung cancer. In embodiments, the cancer is EGFR-mutated non-small cell lung cancer. In embodiments, the DNA Damage Response Inhibitor is an ATM kinase inhibitor, an ATR kinase inhibitor, a Chk1 kinase inhibitor, a Chk2 kinase inhibitor, or a combination of two or more thereof. In embodiments, the DNA Damage Response Inhibitor is an ATM kinase inhibitor. In embodiments, the ATM kinase inhibitor is AZD0156, KU-60019, or AZD1390. In embodiments, the DNA Damage Response Inhibitor is an ATR kinase inhibitor. In embodiments, the ATR kinase inhibitor is M6620 or Bay895344. In embodiments, the DNA Damage Response Inhibitor is a Chk1 kinase inhibitor. In embodiments, the Chk1 kinase inhibitor is SRA737 or Ly2606368. In embodiments, the DNA Damage Response Inhibitor is a Chk2 kinase inhibitor. In embodiments, the Chk2 kinase inhibitor is Ly2606368. In embodiments, the cytotoxic agent is an alkylating agent, an antimetabolite, a mitotic inhibitor, a topoisomerase inhibitor, or a combination of two or more thereof. In embodiments, the cytotoxic agent is an alkylating agent. In embodiments, the alkylating agent is bendamustine, busulfan, carboplatin, carmustine, chlorambucil, cisplatin, cyclophosphamide, decarbazine, estramustine, hydroxyurea, ifosfamide, lomustine, mechlorethamine, melphalan, oxaliplatin, procarbazine, streptozocin, temozolomide, thiotepa, treosulfan, or a combination of two or more thereof. In embodiments, the cytotoxic agent is an antimetabolite. In embodiments, the antimetbolite is azacitidine, capecitabine, cladribine, cytarabine, fludarabine, fluorouracil, gemcitabine, mercaptopurine, methotrexate, pralatrexate, pemetrexed, pentostatin, raltitrexed, thioguanie, trifluridine/tipiracil, or a combination of two or more thereof. In embodiments, the cytotoxic agent is a mitotic inhibitor. In embodiments, the mitotic inhibitor is cabazitaxel, docetaxel, eribulin, ixabepilone, paclitaxel, vinblastine, vincristine, vinorelbine, or a combination of two or more thereof. In embodiments, the cytotoxic agent a topoisomerase inhibitor. In embodiments, the toposiomerase inhibitor is irinotecan, topotecan, amsacrine, daunorubicin, doxorubicin, epirubicin, idarubicin, etoposide, mitoxantrone, teniposide, or a combination of two or more thereof. In embodiments, the cytotoxic agent is aresenic trioxide, asparaginase, bleomycin, belinostat, dactinomycin, iniparib, lurbinectedin, mitomycin, mitotane, porfimer, romidepsin, vorinostat, or a combination of two or more thereof. In embodiments, the methods further comprising administering to the patient a therapeutically effective amount of radiation therapy.

The disclosure provides methods for treating cancer in a patient in need thereof comprising: (i) detecting a 3′ flap Okazaki fragment in a biological sample obtained from the patient; and (ii) administering to the patient a therapeutically effective amount of a DNA Damage Response Inhibitor. In embodiments, the biological sample contains an elevated level of the 3′ flap Okazaki fragment relative to a control. In embodiments, the biological sample is a cancer cell. In embodiments, the biological sample is genomic DNA in a cancer cell. In embodiments, the cancer is leukemia. In embodiments, the cancer is acute lymphoblastic leukemia. In embodiments, the cancer is lung cancer. In embodiments, the cancer is EGFR-mutated lung cancer. In embodiments, the cancer is small cell lung cancer. In embodiments, the cancer is EGFR-mutated small cell lung cancer. In embodiments, the cancer is non-small cell lung cancer. In embodiments, the cancer is EGFR-mutated non-small cell lung cancer. In embodiments, the DNA Damage Response Inhibitor is an ATM kinase inhibitor, an ATR kinase inhibitor, a Chk1 kinase inhibitor, a Chk2 kinase inhibitor, or a combination of two or more thereof. In embodiments, the DNA Damage Response Inhibitor is an ATM kinase inhibitor. In embodiments, the ATM kinase inhibitor is AZD0156, KU-60019, or AZD1390. In embodiments, the DNA Damage Response Inhibitor is an ATR kinase inhibitor. In embodiments, the ATR kinase inhibitor is M6620 or Bay895344. In embodiments, the DNA Damage Response Inhibitor is a Chk1 kinase inhibitor. In embodiments, the Chk1 kinase inhibitor is SRA737 or Ly2606368. In embodiments, the DNA Damage Response Inhibitor is a Chk2 kinase inhibitor. In embodiments, the Chk2 kinase inhibitor is Ly2606368. In embodiments, the methods further comprise administering to the patient a therapeutically effective amount of an anticancer agent. In embodiments, the anticancer agent is a cytotoxic agent. In embodiments, the cytotoxic agent is an alkylating agent, an antimetabolite, a mitotic inhibitor, a topoisomerase inhibitor, or a combination of two or more thereof. In embodiments, the cytotoxic agent is an alkylating agent. In embodiments, the alkylating agent is bendamustine, busulfan, carboplatin, carmustine, chlorambucil, cisplatin, cyclophosphamide, decarbazine, estramustine, hydroxyurea, ifosfamide, lomustine, mechlorethamine, melphalan, oxaliplatin, procarbazine, streptozocin, temozolomide, thiotepa, treosulfan, or a combination of two or more thereof. In embodiments, the cytotoxic agent is an antimetabolite. In embodiments, the antimetbolite is azacitidine, capecitabine, cladribine, cytarabine, fludarabine, fluorouracil, gemcitabine, mercaptopurine, methotrexate, pralatrexate, pemetrexed, pentostatin, raltitrexed, thioguanie, trifluridine/tipiracil, or a combination of two or more thereof. In embodiments, the cytotoxic agent is a mitotic inhibitor. In embodiments, the mitotic inhibitor is cabazitaxel, docetaxel, eribulin, ixabepilone, paclitaxel, vinblastine, vincristine, vinorelbine, or a combination of two or more thereof. In embodiments, the cytotoxic agent a topoisomerase inhibitor. In embodiments, the toposiomerase inhibitor is irinotecan, topotecan, amsacrine, daunorubicin, doxorubicin, epirubicin, idarubicin, etoposide, mitoxantrone, teniposide, or a combination of two or more thereof. In embodiments, the cytotoxic agent is aresenic trioxide, asparaginase, bleomycin, belinostat, dactinomycin, iniparib, lurbinectedin, mitomycin, mitotane, porfimer, romidepsin, vorinostat, or a combination of two or more thereof. In embodiments, the methods further comprising administering to the patient a therapeutically effective amount of radiation therapy. In embodiments, the method of detecting a 3′ flap Okazaki fragment comprises detecting an alternative duplication formed by the 3′ flap Okazaki fragment.

The disclosure provides methods for treating cancer in a patient in need thereof comprising: (i) detecting a 3′ flap Okazaki fragment in a biological sample obtained from the patient; and (ii) administering to the patient a therapeutically effective amount of a DNA Damage Response Inhibitor and a therapeutically effective amount of a cytotoxic agent. In embodiments, the biological sample contains an elevated level of the 3′ flap Okazaki fragment relative to a control. In embodiments, the biological sample is a cancer cell. In embodiments, the biological sample is genomic DNA in a cancer cell. In embodiments, the cancer is leukemia. In embodiments, the cancer is acute lymphoblastic leukemia. In embodiments, the cancer is lung cancer. In embodiments, the cancer is EGFR-mutated lung cancer. In embodiments, the cancer is small cell lung cancer. In embodiments, the cancer is EGFR-mutated small cell lung cancer. In embodiments, the cancer is non-small cell lung cancer. In embodiments, the cancer is EGFR-mutated non-small cell lung cancer. In embodiments, the DNA Damage Response Inhibitor is an ATM kinase inhibitor, an ATR kinase inhibitor, a Chk1 kinase inhibitor, a Chk2 kinase inhibitor, or a combination of two or more thereof. In embodiments, the DNA Damage Response Inhibitor is an ATM kinase inhibitor. In embodiments, the ATM kinase inhibitor is AZD0156, KU-60019, or AZD1390. In embodiments, the DNA Damage Response Inhibitor is an ATR kinase inhibitor. In embodiments, the ATR kinase inhibitor is M6620 or Bay895344. In embodiments, the DNA Damage Response Inhibitor is a Chk1 kinase inhibitor. In embodiments, the Chk1 kinase inhibitor is SRA737 or Ly2606368. In embodiments, the DNA Damage Response Inhibitor is a Chk2 kinase inhibitor. In embodiments, the Chk2 kinase inhibitor is Ly2606368. In embodiments, the cytotoxic agent is an alkylating agent, an antimetabolite, a mitotic inhibitor, a topoisomerase inhibitor, or a combination of two or more thereof. In embodiments, the cytotoxic agent is an alkylating agent. In embodiments, the alkylating agent is bendamustine, busulfan, carboplatin, carmustine, chlorambucil, cisplatin, cyclophosphamide, decarbazine, estramustine, hydroxyurea, ifosfamide, lomustine, mechlorethamine, melphalan, oxaliplatin, procarbazine, streptozocin, temozolomide, thiotepa, treosulfan, or a combination of two or more thereof. In embodiments, the cytotoxic agent is an antimetabolite. In embodiments, the antimetbolite is azacitidine, capecitabine, cladribine, cytarabine, fludarabine, fluorouracil, gemcitabine, mercaptopurine, methotrexate, pralatrexate, pemetrexed, pentostatin, raltitrexed, thioguanie, trifluridine/tipiracil, or a combination of two or more thereof. In embodiments, the cytotoxic agent is a mitotic inhibitor. In embodiments, the mitotic inhibitor is cabazitaxel, docetaxel, eribulin, ixabepilone, paclitaxel, vinblastine, vincristine, vinorelbine, or a combination of two or more thereof. In embodiments, the cytotoxic agent a topoisomerase inhibitor. In embodiments, the toposiomerase inhibitor is irinotecan, topotecan, amsacrine, daunorubicin, doxorubicin, epirubicin, idarubicin, etoposide, mitoxantrone, teniposide, or a combination of two or more thereof. In embodiments, the cytotoxic agent is aresenic trioxide, asparaginase, bleomycin, belinostat, dactinomycin, iniparib, lurbinectedin, mitomycin, mitotane, porfimer, romidepsin, vorinostat, or a combination of two or more thereof. In embodiments, the methods further comprising administering to the patient a therapeutically effective amount of radiation therapy. In embodiments, the method of detecting a 3′ flap Okazaki fragment comprises detecting an alternative duplication formed by the 3′ flap Okazaki fragment.

The disclosure provides methods for identifying a cancer patient in need of treatment with a DNA Damage Response Inhibitor comprising detecting a 3′ flap Okazaki fragment in a biological sample obtained from the patient; wherein the presence of the 3′ flap Okazaki fragment indicates that the cancer patient is in need of treatment with the DNA Damage Response Inhibitor. In embodiments, the method further comprises administering to the patient a therapeutically effective amount of a DNA Damage Response Inhibitor. In embodiments, the biological sample contains an elevated level of the 3′ flap Okazaki fragment relative to a control. In embodiments, the biological sample is a cancer cell. In embodiments, the biological sample is genomic DNA in a cancer cell. In embodiments, the cancer is leukemia. In embodiments, the cancer is acute lymphoblastic leukemia. In embodiments, the cancer is lung cancer. In embodiments, the cancer is EGFR-mutated lung cancer. In embodiments, the cancer is small cell lung cancer. In embodiments, the cancer is EGFR-mutated small cell lung cancer. In embodiments, the cancer is non-small cell lung cancer. In embodiments, the cancer is EGFR-mutated non-small cell lung cancer. In embodiments, the DNA Damage Response Inhibitor is an ATM kinase inhibitor, an ATR kinase inhibitor, a Chk1 kinase inhibitor, a Chk2 kinase inhibitor, or a combination of two or more thereof. In embodiments, the DNA Damage Response Inhibitor is an ATM kinase inhibitor. In embodiments, the ATM kinase inhibitor is AZD0156, KU-60019, or AZD1390. In embodiments, the DNA Damage Response Inhibitor is an ATR kinase inhibitor. In embodiments, the ATR kinase inhibitor is M6620 or Bay895344. In embodiments, the DNA Damage Response Inhibitor is a Chk1 kinase inhibitor. In embodiments, the Chk1 kinase inhibitor is SRA737 or Ly2606368. In embodiments, the DNA Damage Response Inhibitor is a Chk2 kinase inhibitor. In embodiments, the Chk2 kinase inhibitor is Ly2606368. In embodiments, the methods further comprise administering to the patient a therapeutically effective amount of an anticancer agent. In embodiments, the anticancer agent is a cytotoxic agent. In embodiments, the cytotoxic agent is an alkylating agent, an antimetabolite, a mitotic inhibitor, a topoisomerase inhibitor, or a combination of two or more thereof. In embodiments, the cytotoxic agent is an alkylating agent. In embodiments, the alkylating agent is bendamustine, busulfan, carboplatin, carmustine, chlorambucil, cisplatin, cyclophosphamide, decarbazine, estramustine, hydroxyurea, ifosfamide, lomustine, mechlorethamine, melphalan, oxaliplatin, procarbazine, streptozocin, temozolomide, thiotepa, treosulfan, or a combination of two or more thereof. In embodiments, the cytotoxic agent is an antimetabolite. In embodiments, the antimetbolite is azacitidine, capecitabine, cladribine, cytarabine, fludarabine, fluorouracil, gemcitabine, mercaptopurine, methotrexate, pralatrexate, pemetrexed, pentostatin, raltitrexed, thioguanie, trifluridine/tipiracil, or a combination of two or more thereof. In embodiments, the cytotoxic agent is a mitotic inhibitor. In embodiments, the mitotic inhibitor is cabazitaxel, docetaxel, eribulin, ixabepilone, paclitaxel, vinblastine, vincristine, vinorelbine, or a combination of two or more thereof. In embodiments, the cytotoxic agent a topoisomerase inhibitor. In embodiments, the toposiomerase inhibitor is irinotecan, topotecan, amsacrine, daunorubicin, doxorubicin, epirubicin, idarubicin, etoposide, mitoxantrone, teniposide, or a combination of two or more thereof. In embodiments, the cytotoxic agent is aresenic trioxide, asparaginase, bleomycin, belinostat, dactinomycin, iniparib, lurbinectedin, mitomycin, mitotane, porfimer, romidepsin, vorinostat, or a combination of two or more thereof. In embodiments, the methods further comprising administering to the patient a therapeutically effective amount of radiation therapy. In embodiments, the method of detecting a 3′ flap Okazaki fragment comprises detecting an alternative duplication formed by the 3′ flap Okazaki fragment.

The disclosure provides methods for diagnosing a patient with cancer comprising detecting a 3′ flap Okazaki fragment in a biological sample obtained from the patient; wherein the presence of the 3′ flap Okazaki fragment indicates that the patient has cancer. In embodiments, the method further comprises administering to the patient a therapeutically effective amount of a DNA Damage Response Inhibitor. In embodiments, the biological sample contains an elevated level of the 3′ flap Okazaki fragment relative to a control. In embodiments, the biological sample is a cancer cell. In embodiments, the biological sample is genomic DNA in a cancer cell. In embodiments, the cancer is leukemia. In embodiments, the cancer is acute lymphoblastic leukemia. In embodiments, the cancer is lung cancer. In embodiments, the cancer is EGFR-mutated lung cancer. In embodiments, the cancer is small cell lung cancer. In embodiments, the cancer is EGFR-mutated small cell lung cancer. In embodiments, the cancer is non-small cell lung cancer. In embodiments, the cancer is EGFR-mutated non-small cell lung cancer. In embodiments, the DNA Damage Response Inhibitor is an ATM kinase inhibitor, an ATR kinase inhibitor, a Chk1 kinase inhibitor, a Chk2 kinase inhibitor, or a combination of two or more thereof. In embodiments, the DNA Damage Response Inhibitor is an ATM kinase inhibitor. In embodiments, the ATM kinase inhibitor is AZD0156, KU-60019, or AZD1390. In embodiments, the DNA Damage Response Inhibitor is an ATR kinase inhibitor. In embodiments, the ATR kinase inhibitor is M6620 or Bay895344. In embodiments, the DNA Damage Response Inhibitor is a Chk1 kinase inhibitor. In embodiments, the Chk1 kinase inhibitor is SRA737 or Ly2606368. In embodiments, the DNA Damage Response Inhibitor is a Chk2 kinase inhibitor. In embodiments, the Chk2 kinase inhibitor is Ly2606368. In embodiments, the methods further comprise administering to the patient a therapeutically effective amount of an anticancer agent. In embodiments, the anticancer agent is a cytotoxic agent. In embodiments, the cytotoxic agent is an alkylating agent, an antimetabolite, a mitotic inhibitor, a topoisomerase inhibitor, or a combination of two or more thereof. In embodiments, the cytotoxic agent is an alkylating agent. In embodiments, the alkylating agent is bendamustine, busulfan, carboplatin, carmustine, chlorambucil, cisplatin, cyclophosphamide, decarbazine, estramustine, hydroxyurea, ifosfamide, lomustine, mechlorethamine, melphalan, oxaliplatin, procarbazine, streptozocin, temozolomide, thiotepa, treosulfan, or a combination of two or more thereof. In embodiments, the cytotoxic agent is an antimetabolite. In embodiments, the antimetbolite is azacitidine, capecitabine, cladribine, cytarabine, fludarabine, fluorouracil, gemcitabine, mercaptopurine, methotrexate, pralatrexate, pemetrexed, pentostatin, raltitrexed, thioguanie, trifluridine/tipiracil, or a combination of two or more thereof. In embodiments, the cytotoxic agent is a mitotic inhibitor. In embodiments, the mitotic inhibitor is cabazitaxel, docetaxel, eribulin, ixabepilone, paclitaxel, vinblastine, vincristine, vinorelbine, or a combination of two or more thereof. In embodiments, the cytotoxic agent a topoisomerase inhibitor. In embodiments, the toposiomerase inhibitor is irinotecan, topotecan, amsacrine, daunorubicin, doxorubicin, epirubicin, idarubicin, etoposide, mitoxantrone, teniposide, or a combination of two or more thereof. In embodiments, the cytotoxic agent is aresenic trioxide, asparaginase, bleomycin, belinostat, dactinomycin, iniparib, lurbinectedin, mitomycin, mitotane, porfimer, romidepsin, vorinostat, or a combination of two or more thereof. In embodiments, the methods further comprising administering to the patient a therapeutically effective amount of radiation therapy. In embodiments, the method of detecting a 3′ flap Okazaki fragment comprises detecting an alternative duplication formed by the 3′ flap Okazaki fragment.

### Embodiments 1 to 34

Embodiment 1. A method for treating cancer in a patient in need thereof, the method comprising administering to the patient a therapeutically effective amount of a DNA Damage Response Inhibitor; wherein a biological sample obtained from the patient contains a 3′ flap Okazaki fragment.

Embodiment 2. A method for treating cancer in a patient in need thereof, the method comprising: (i) detecting a 3′ flap Okazaki fragment in a biological sample obtained from the patient; and (ii) administering to the patient a therapeutically effective amount of a DNA Damage Response Inhibitor.

Embodiment 3. A method for identifying a cancer patient in need of treatment with a DNA Damage Response Inhibitor, the method comprising detecting a 3′ flap Okazaki fragment in a biological sample obtained from the patient; wherein the presence of the 3′ flap Okazaki fragment indicates that the cancer patient is in need of treatment with the DNA Damage Response Inhibitor.

Embodiment 4. A method for diagnosing a patient with cancer, the method comprising detecting a 3′ flap Okazaki fragment in a biological sample obtained from the patient; wherein the presence of the 3′ flap Okazaki fragment indicates that the patient has cancer.

Embodiment 5. The method of Embodiment 3 or 4, further comprising administering to the patient a therapeutically effective amount of a DNA Damage Response Inhibitor.

Embodiment 6. The method of any one of Embodiments 1 to 5, wherein the biological sample contains an elevated level of the 3′ flap Okazaki fragment relative to a control.

Embodiment 7. The method of any one of Embodiments 1 to 6, wherein the biological sample is a cancer cell.

Embodiment 8. The method of any one of Embodiments 1 to 7, wherein the cancer is leukemia.

Embodiment 9. The method of any one of Embodiments 1 to 7, wherein the cancer is acute lymphoblastic leukemia.

Embodiment 10. The method of any one of Embodiments 1 to 7, wherein the cancer is lung cancer.

Embodiment 11. The method of Embodiment 10, wherein the lung cancer is EGFR-mutated lung cancer.

Embodiment 12. The method of Embodiment 10, wherein the lung cancer is small cell lung cancer.

Embodiment 13. The method of Embodiment 10, wherein the lung cancer is EGFR-mutated small cell lung cancer.

Embodiment 14. The method of Embodiment 10, wherein the lung cancer is non-small cell lung cancer.

Embodiment 15. The method of Embodiment 10, wherein the lung cancer is EGFR-mutated non-small cell lung cancer.

Embodiment 16. The method of any one of Embodiments 1, 2, and 5-15, wherein the DNA Damage Response Inhibitor is an ATM kinase inhibitor, an ATR kinase inhibitor, a Chk1 kinase inhibitor, a Chk2 kinase inhibitor, or a combination of two or more thereof.

Embodiment 17. The method of Embodiment 16, wherein the ATM kinase inhibitor is AZD0156, KU-60019, or AZD1390.

Embodiment 18. The method of Embodiment 16, wherein the ATR kinase inhibitor is berzosertib or elimusertib.

Embodiment 19. The method of Embodiment 16, wherein the Chk1 kinase inhibitor is SRA737 or prexasertib.

Embodiment 20. The method of Embodiment 16, wherein the Chk2 kinase inhibitor is Ly2606368.

Embodiment 21. The method of any one of Embodiments 1 to 20, further comprising administering to the patient a therapeutically effective amount of an anticancer agent.

Embodiment 22. The method of Embodiment 21, wherein the anticancer agent is a cytotoxic agent.

Embodiment 23. The method of Embodiment 22, wherein the cytotoxic agent is an alkylating agent, an antimetabolite, a mitotic inhibitor, a topoisomerase inhibitor, or a combination of two or more thereof.

Embodiment 24. The method of Embodiment 22, wherein the cytotoxic agent is bendamustine, busulfan, carboplatin, carmustine, chlorambucil, cisplatin, cyclophosphamide, decarbazine, estramustine, hydroxyurea, ifosfamide, lomustine, mechlorethamine, melphalan, oxaliplatin, procarbazine, streptozocin, temozolomide, thiotepa, treosulfan, azacitidine, capecitabine, cladribine, cytarabine, fludarabine, fluorouracil, gemcitabine, mercaptopurine, methotrexate, pralatrexate, pemetrexed, pentostatin, raltitrexed, thioguanie, trifluridine/tipiracil, cabazitaxel, docetaxel, eribulin, ixabepilone, paclitaxel, vinblastine, vincristine, vinorelbine, irinotecan, topotecan, amsacrine, daunorubicin, doxorubicin, epirubicin, idarubicin, etoposide, mitoxantrone, teniposide, aresenic trioxide, asparaginase, bleomycin, belinostat, dactinomycin, iniparib, lurbinectedin, mitomycin, mitotane, porfimer, romidepsin, vorinostat, or a combination of two or more thereof.

Embodiment 25. The method of any one of Embodiments 1 to 20, further comprising administering to the patient a therapeutically effective amount of an alkylating agent.

Embodiment 26. The method of Embodiment 25, wherein the alkylating agent is bendamustine, busulfan, carboplatin, carmustine, chlorambucil, cisplatin, cyclophosphamide, decarbazine, estramustine, hydroxyurea, ifosfamide, lomustine, mechlorethamine, melphalan, oxaliplatin, procarbazine, streptozocin, temozolomide, thiotepa, treosulfan, or a combination of two or more thereof.

Embodiment 27. The method of any one of Embodiments 1 to 20, further comprising administering to the patient a therapeutically effective amount of an antimetabolite.

Embodiment 28. The method Embodiment 25, wherein the antimetbolite is azacitidine, capecitabine, cladribine, cytarabine, fludarabine, fluorouracil, gemcitabine, mercaptopurine, methotrexate, pralatrexate, pemetrexed, pentostatin, raltitrexed, thioguanie, trifluridine/tipiracil, or a combination of two or more thereof.

Embodiment 29. The method of any one of Embodiments 1 to 20, further comprising administering to the patient a therapeutically effective amount of a mitotic inhibitor.

Embodiment 30. The method of Embodiment 29, wherein the mitotic inhibitor is cabazitaxel, docetaxel, eribulin, ixabepilone, paclitaxel, vinblastine, vincristine, vinorelbine, or a combination of two or more thereof.

Embodiment 31. The method of any one of Embodiments 1 to 20, further comprising administering to the patient a therapeutically effective amount of a topoisomerase inhibitor.

Embodiment 32. The method of Embodiment 31, wherein the toposiomerase inhibitor is irinotecan, topotecan, amsacrine, daunorubicin, doxorubicin, epirubicin, idarubicin, etoposide, mitoxantrone, teniposide, or a combination of two or more thereof.

Embodiment 33. The method of any one of Embodiments 1 to 20, further comprising administering to the patient a therapeutically effective amount of aresenic trioxide, asparaginase, bleomycin, belinostat, dactinomycin, iniparib, lurbinectedin, mitomycin, mitotane, porfimer, romidepsin, vorinostat, or a combination of two or more thereof.

Embodiment 34. The method of any one of Embodiments 1 to 33, further comprising administering to the patient a therapeutically effective amount of radiation therapy.

### EXAMPLES

How cells with DNA replication defects acquire mutations that allow them to escape apoptosis under environmental stress is a long-standing question. Here, we report an error-prone Okazaki fragment maturation (OFM) pathway that is activated at restrictive temperatures in rad27Δ yeast cells. Restrictive temperature stress activates Dun1, facilitating transformation of un-processed 5′ flaps into 3′ flaps, which are removed by 3′ nucleases including Polδ. However, at certain regions, 3′ flaps form secondary structures that facilitate 3′ end extension rather than degradation, producing alternative duplications with short spacer sequences. Once such mutations occur at POL3, it fails to displace 5′flaps, thus rescues rad27Δ cells. Our study defines a stress-induced, error-prone OFM pathway that generates mutations that counteract replication defects and drive cellular evolution and survival. This discovery can be readily translated to cancer treatment and diagnosis in humans.

Understanding the mutagenesis mechanisms that are active in cells under stress conditions is crucial for developing strategies to intervene in microbial pathogenesis, tumorigenesis, and drug resistance (1, 2). Lagging-strand DNA synthesis is particularly vulnerable to stress and environmental factors. During replication, lagging-strand DNA is synthesized as discrete Okazaki fragments (3), which contain short primase/Polα-synthesized RNA-DNA primers at their 5′ ends (4-6). During Okazaki fragment maturation (OFM), the RNA portion and any Polα-synthesized DNA with high incorporation errors are removed, via Polδ-mediated displacement DNA synthesis, which produces a 5′ RNA-DNA flap (4-6). The 5′ flap structure is removed by flap endonuclease 1 (FEN1) or through the sequential actions of DNA2 nuclease/helicase and FEN1 (7-9). FEN1 deficiency leads to accumulation of unprocessed 5′ flap structures, which may prevent ligation of Okazaki fragments, leaving DNA nicks or gaps that lead to collapse of replication forks and DNA double-strand breaks. In yeast, deletion of the FEN1 homolog RAD27 (rad27Δ) results in slow growth at permissive growth temperatures (30° C.) and death at restrictive growth temperatures (37° C.) (10).

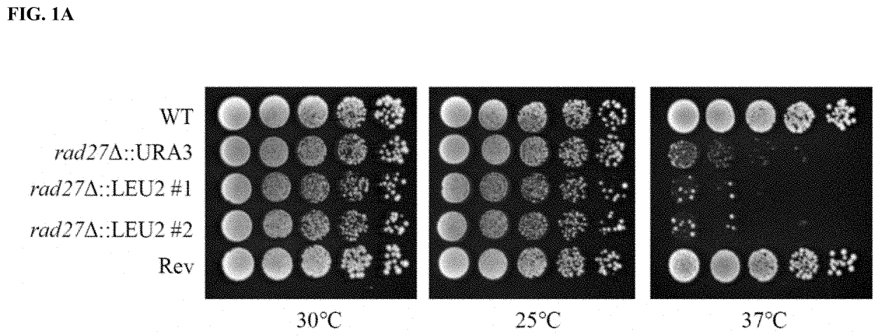

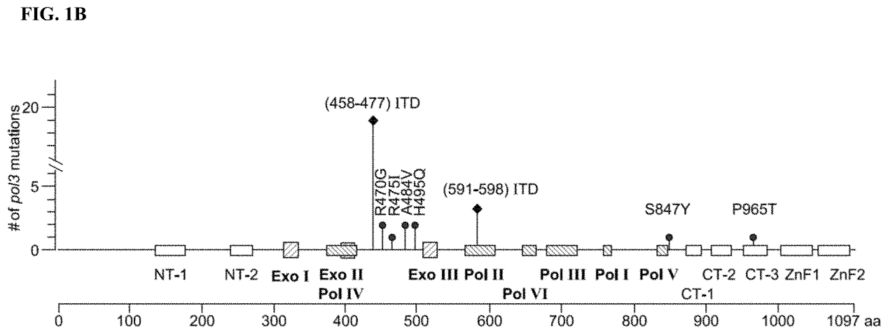

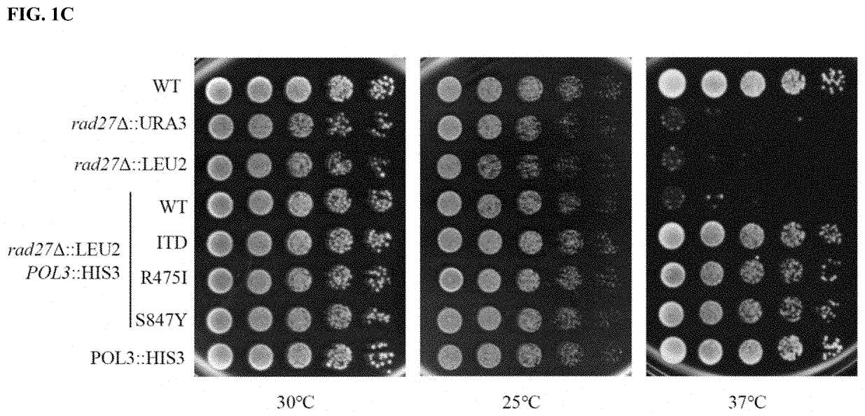

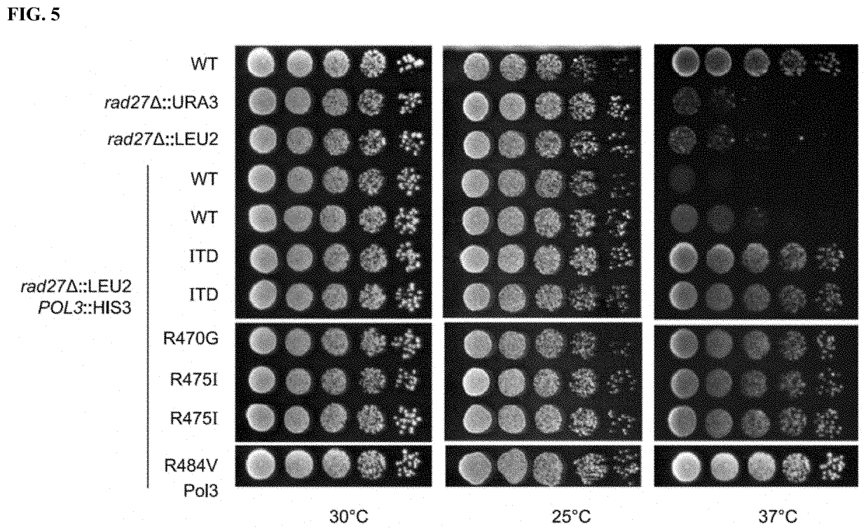

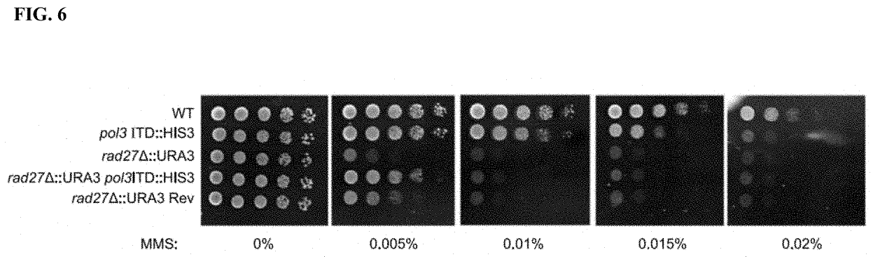

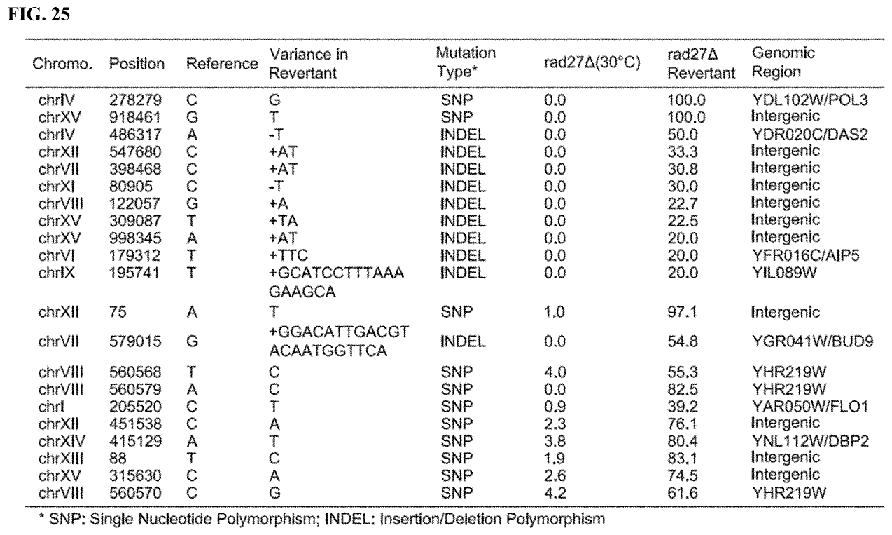

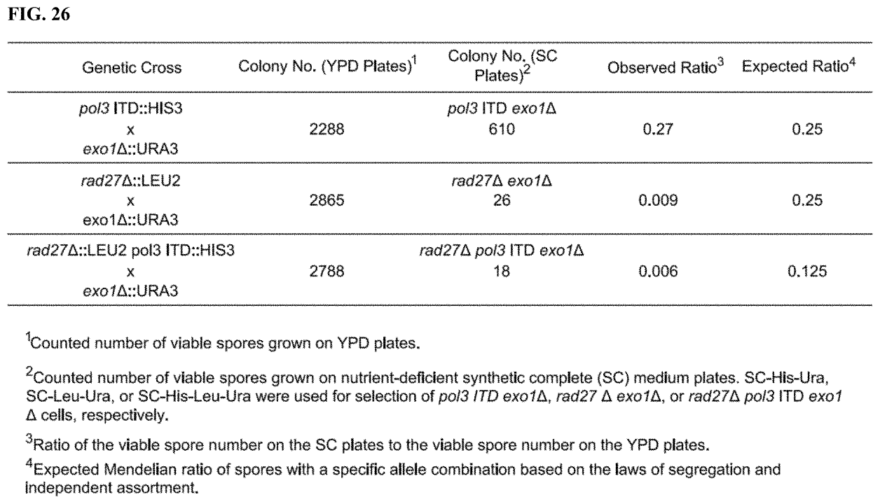

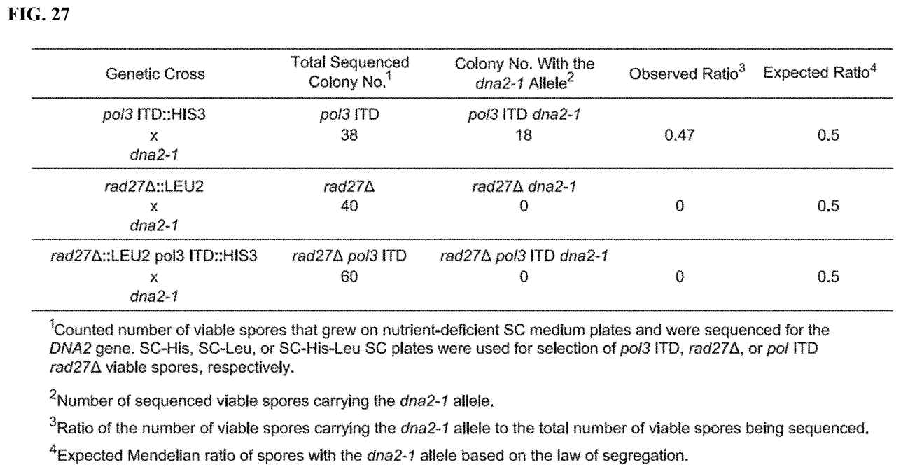

Nevertheless, we discovered that a small population of rad27Δ yeast cells, which we called revertants, could grow at a similar rate as wild-type (WT) cells at 37° C. (FIG. 1A). To determine if the revertants acquired somatic mutation(s) that permitted growth and to identify any such mutation(s), we conducted whole-genome sequencing (WGS) of WT, parental rad27Δ, and a revertant strain of yeast cells. We identified 21 somatic DNA mutations specific to one revertant colony (FIG. 25). A mutation in POL3, the DNA polymerase delta (Polδ) catalytic subunit (11), was the only nonsynonymous mutation that had 100% allele frequency in the revertant. Subsequent DNA sequencing analysis of the POL3 gene in independent rad27Δ revertant colonies (n=31) revealed that each colony harbored a pol3 mutation (FIG. 1B). This indicates that these pol3 mutations, which map onto POL3 functional motifs (FIG. 1B, Supplementary text S1) and possibly affect its biochemical activities, provide a survival advantage for rad27Δ cells grown under restrictive temperature stress. Furthermore, knock-in of the 458-477 internal tandem duplication (ITD) mutation, which occurred in 19 of the 31 independent colonies, or any of the four representative point mutations (R470G, R475I, A484V, and S847Y) successfully reversed the restrictive temperature-induced lethality phenotype of rad27Δ cells (FIGS. 1C and 5). rad27Δ cells are sensitive to methyl methanesulfonate (MMS) (10). Although rad27Δ revertant cells and rad27Δ pol3 ITD knock-in mutant cells were resistant to a low concentration (0.005%) of MMS, they were sensitive to higher concentrations (≥0.01%) of MMS (FIG. 6). We observed that pol3 ITD cells in a WT RAD27 background were also sensitive to high concentrations of MMS (FIG. 6). This at least partially explains why the pol3 ITD could not suppress MMS-induced lethality of rad27Δ cells at high MMS concentrations. In addition, pol3 ITD did not rescue the synthetic lethality that occurs in the context of rad27Δ coupled with deficiency of the 5′ nucleases EXO1 or DNA2 nuclease/helicase (FIGS. 26-27, Supplementary text S2).

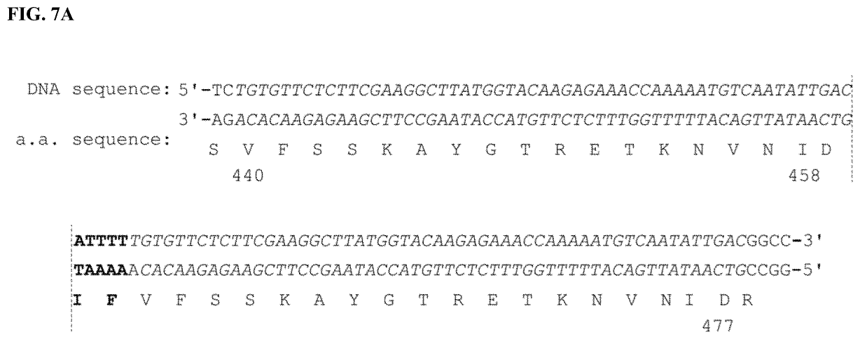

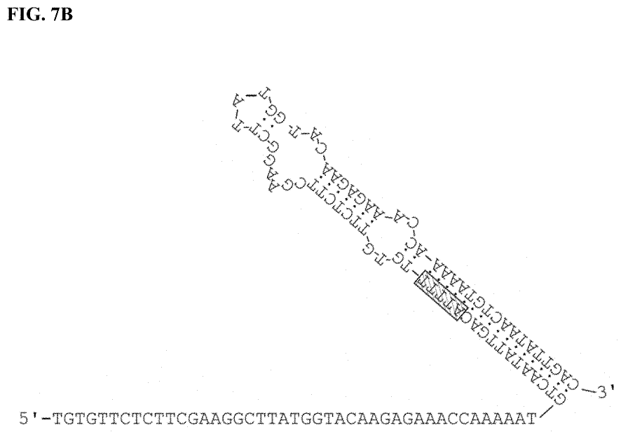

Two types of duplications were present in the revertants: pol3 591-598 ITD, a previously reported classic duplication resulting from re-alignment and ligation of unprocessed 5′ flaps (12), and pol3 458-477 ITD, which contained a 55 bp duplication with a 5 bp spacer between the duplicated units (FIG. 7). We named the duplication with an intervening spacer an “alternative duplication.” Both pol3 591-598 ITD and pol3 458-477 ITD resembled ITDs detected in human cancer (13-15). To determine how the alternative duplication pol3 458-477 ITD originated, we conducted WGS of WT and rad27Δ cells grown at 37° C. or 30° C. for 4 hours. The mutation frequency of WT cells was the same at both temperatures (FIG. 2A). In contrast, restrictive temperature stress increased the mutation frequency of rad27Δ cells by 2-fold; in particular, the frequency of duplications and base substitutions was increased (FIG. 2A). In addition, duplication insertions in rad27Δ cells grown at 37° C. were considerably longer than those in rad27Δ cells grown at 30° C. (FIG. 2B). The duplications revealed that rad27Δ cells grown at 37° C. exhibited alternative duplications that were similar to the pol3 458-477 ITD. The alternative duplications were not detected in WT cells (30° C. or 37° C.) or in rad27Δ cells grown at 30° C. (FIG. 2C), indicating that alternative duplications were induced by restrictive temperature stress.

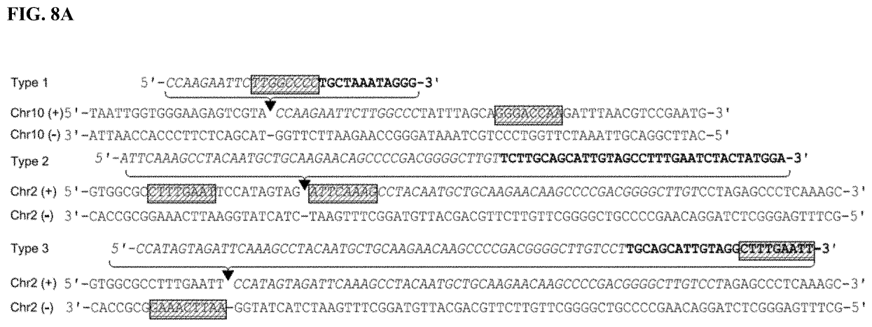

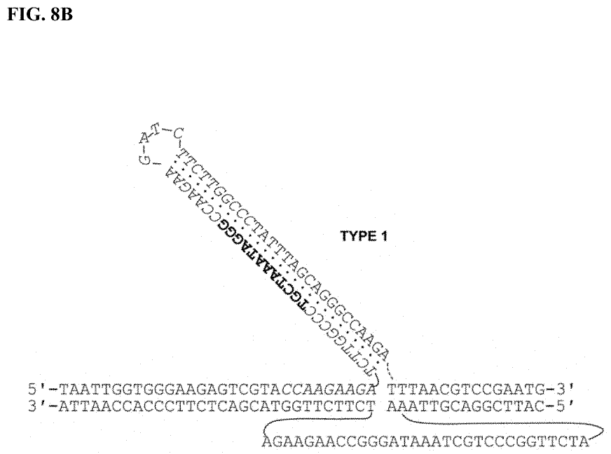

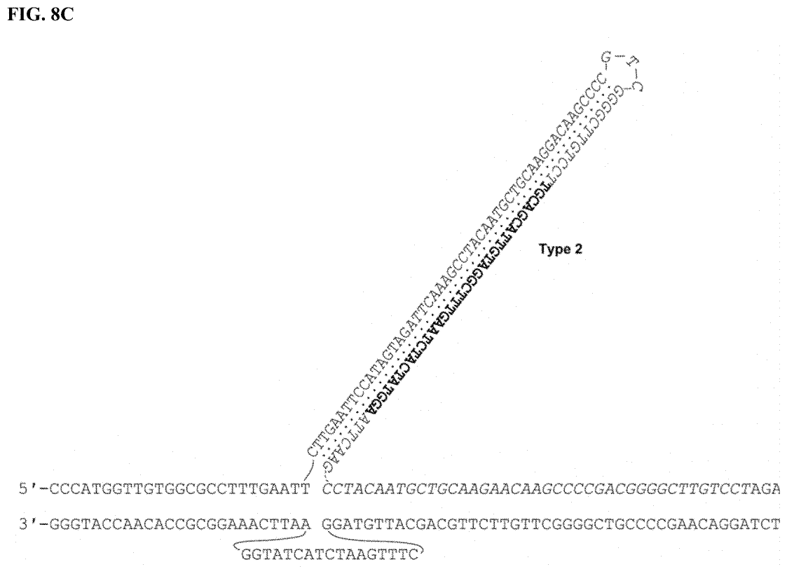

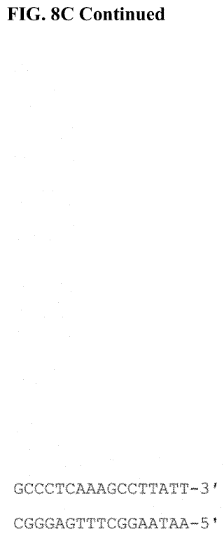

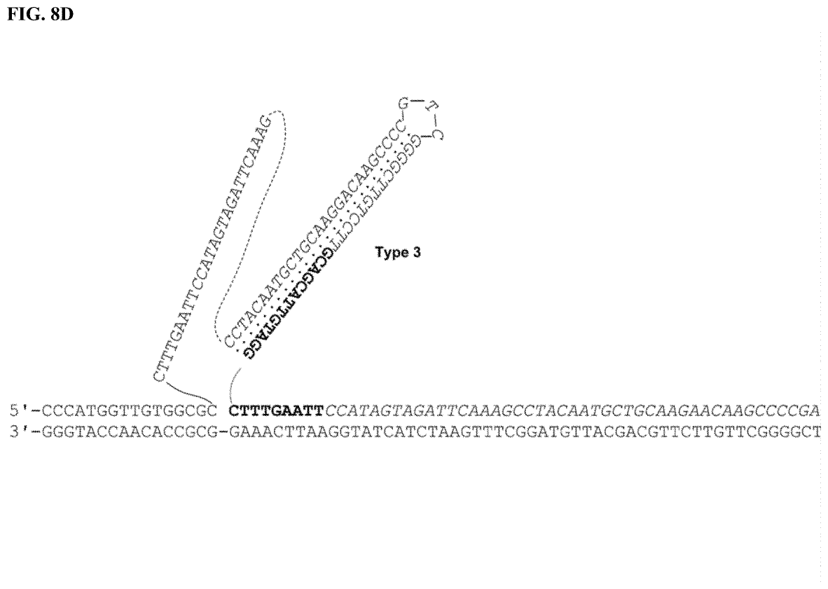

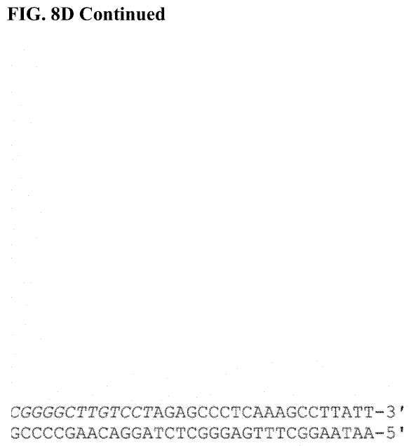

We further noted that the sequences of these alternative duplications indicated formation of three different types of hairpin structures (FIGS. 2D, 8A-8D, Supplementary text S3). This supports a model of sequential actions, including conversion of a 5′ Okazaki fragment flap to a 3′ flap, annealing of the flap to a complementary sequence, extension of the 3′ flap, realignment, and ligation of the extended 3′ flap to produce an alternative duplication, including pol3 458-477 ITD. Consistent with this model, our WGS data indicated that 40% of the alternative duplications also carried base substitutions at the duplication unit. These substitutions most likely resulted from failure to remove Polα-generated errors on the 5′ flap. To determine if the restrictive temperature induced 3′ flap formation in rad27Δ cells, we developed an approach to specifically label the OH group on the 3′ flap on genomic DNA, in which 3′ OH at the nick or at the DNA end was pre-blocked with dideoxyribonucleotides (FIG. 2E). We detected a considerable number of 3′ flaps in rad27Δ cells grown at 37° C.; in contrast, we detected few flaps in rad27Δ cells grown at 30° C., in WT cells grown at either temperature, or in rad27Δ cells carrying pol3 458-477 ITD grown at either temperature (FIG. 2F). Furthermore, pre-incubation of Polδ with genomic DNA from rad27Δ cells grown at 37° C. could effectively remove the 3′ flaps (FIG. 2G), indicating that Polδ processes 3′ flaps during OFM.

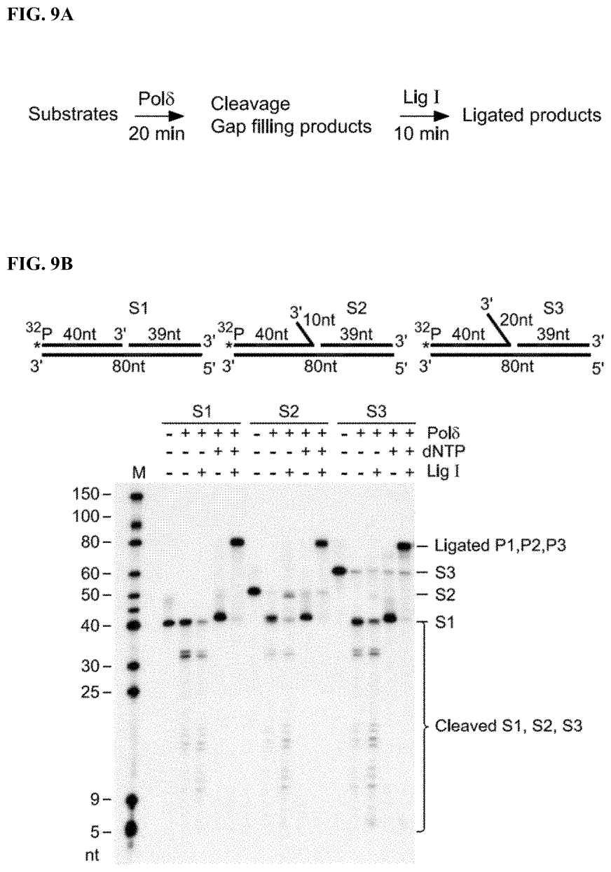

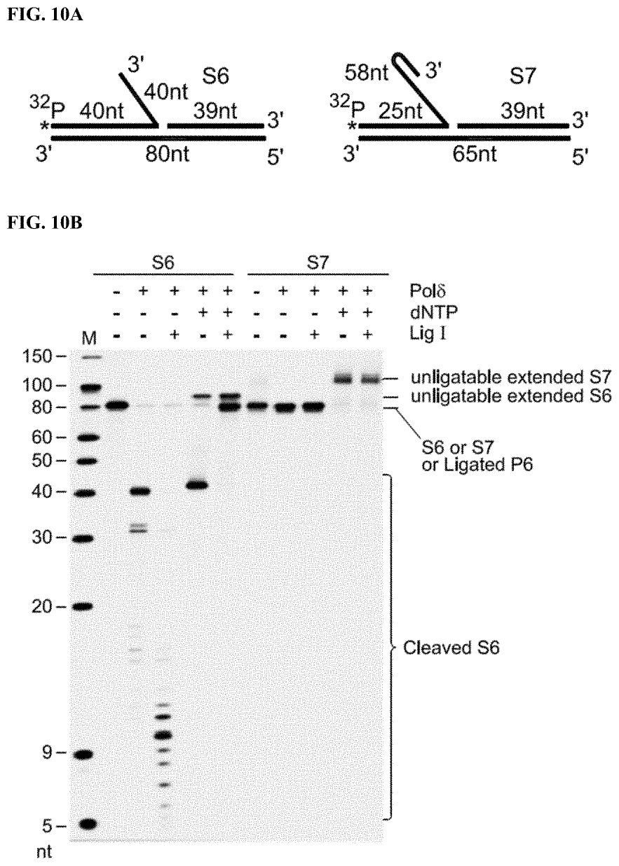

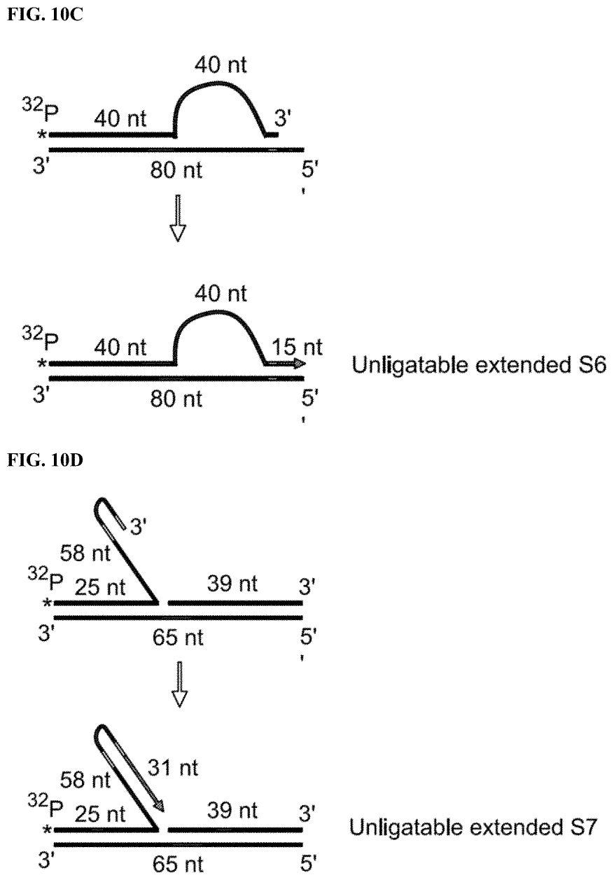

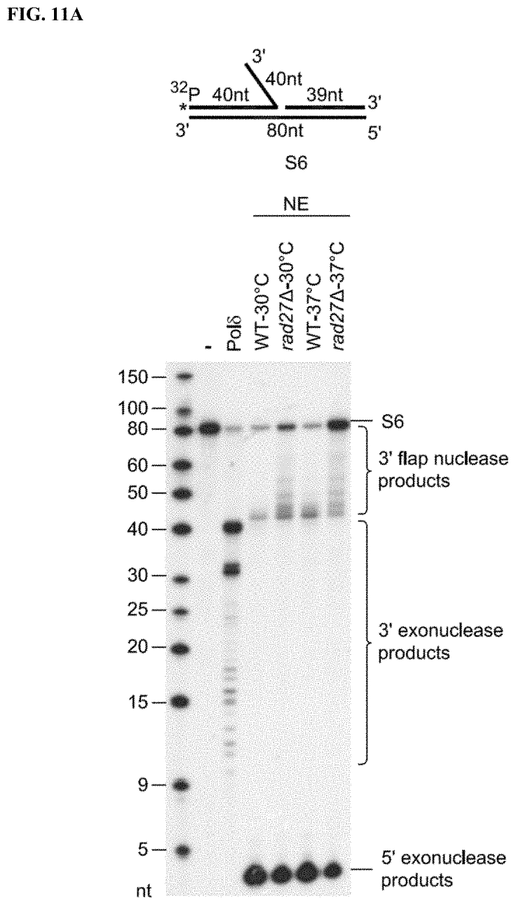

To define the proposed 3′ flap-based OFM mechanism, we reconstituted the sequential reactions of 3′ flap cleavage, DNA synthesis, and ligation of oligo-based DNA substrates (S) with a simple 3′ flap (S2 or S3; FIGS. 2H, FIG. 9B) or a secondary structure-forming 3′ flap (S4 or S5; FIG. 2I) for formation of type I or type II alternative duplications. In the presence of deoxyribonucleotide, Polδ could effectively cleave 3′ flap substrates S2 and S3 and stop at the junction of the 3′ flap and DNA duplex, generating ligatable DNA nicks for DNA Lig I (FIGS. 2H, 9, Supplementary text S4). However, deoxyribonucleotides inhibited cleavage of hairpin-forming 3′ flaps, and promoted extension of the annealed 3′ flap, producing ligated extended products (FIG. 2I, Supplementary text S4); this process resembled formation of alternative duplications. When extension of the annealed 3′ flap could not generate ligatable nicks, only unligatable extended products were produced (FIG. 10A-10D), leading to failure of 3′ flap-based OFM. The single-stranded DNA (ssDNA) binding protein RPA had little effect on Polδ-mediated 3′ flap cleavage or subsequent nick ligation (FIG. 2H), and it slightly enhanced formation of the ligated extended products (FIG. 2I).

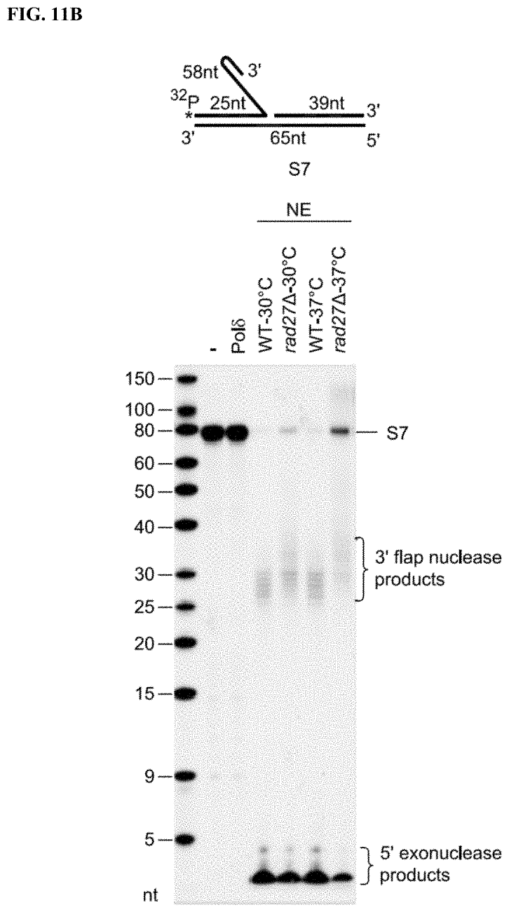

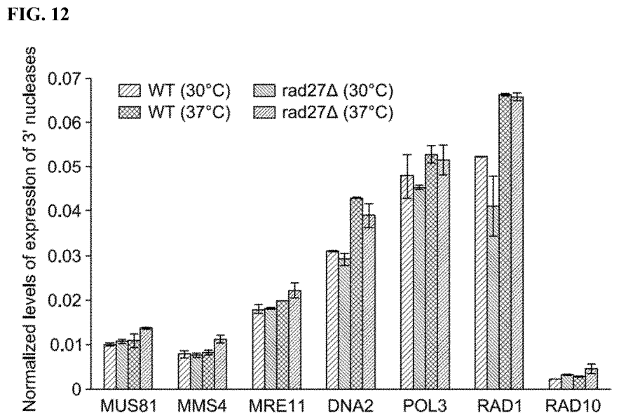

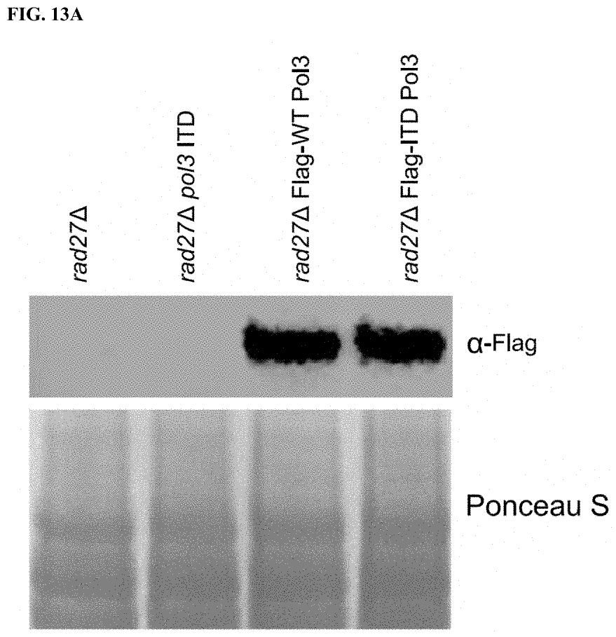

Using reconstitution assays, we showed that the 3′ nuclease activities of Polδ and Lig I were sufficient to complete 3′ flap processing for OFM. Other nucleases in the nuclear extract (NE) might also be important in processing 3′ flaps, especially the hairpin-forming 3′ flap (FIGS. 11A-11B, Supplementary text S5). However, NE from rad27Δ cells, particularly those grown at 37° C., had reduced 3′ flap processing activity (FIGS. 11A-11B, Supplementary text S5). Because we observed no significant changes in expression of major 3′ nucleases in yeast (FIG. 12), we postulated that restrictive temperature stress could also induce molecular changes to inhibit 3′ flap processing, allowing 3′ flaps to invade into nearby homologous sequences, leading to alternative duplications.

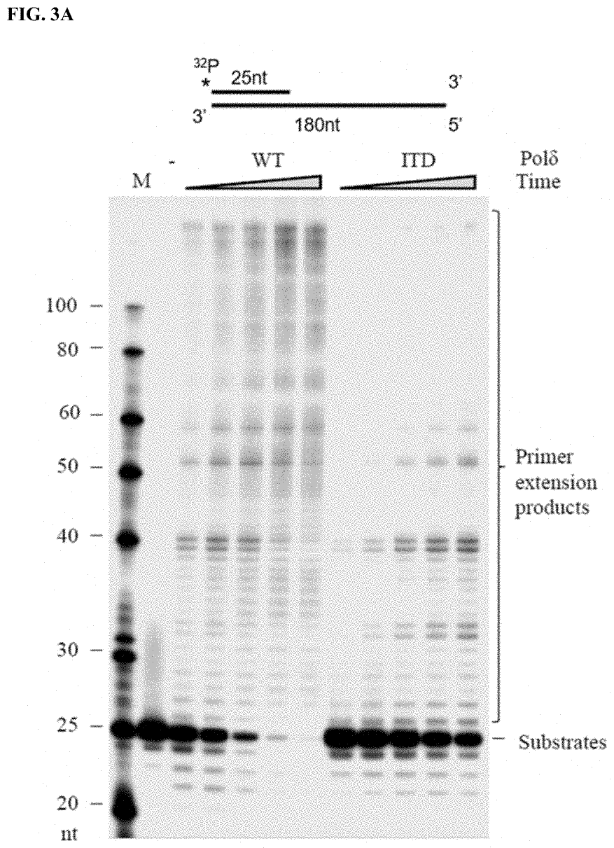

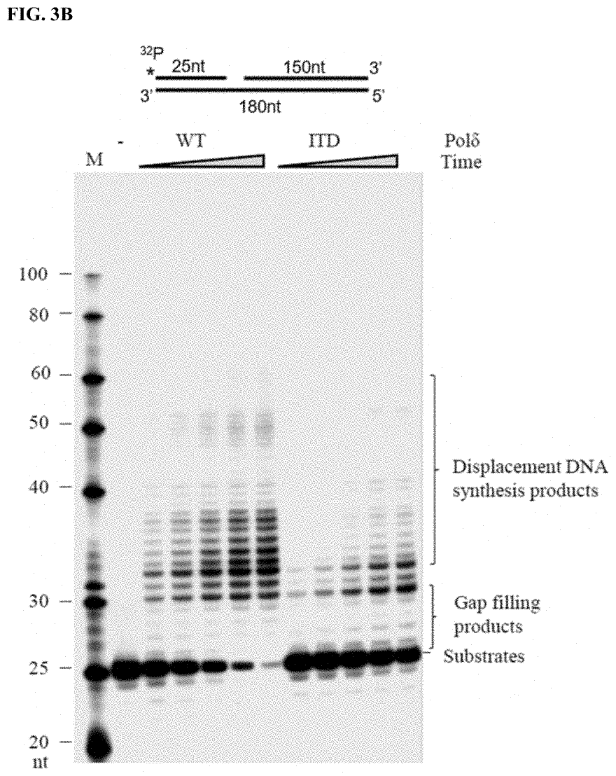

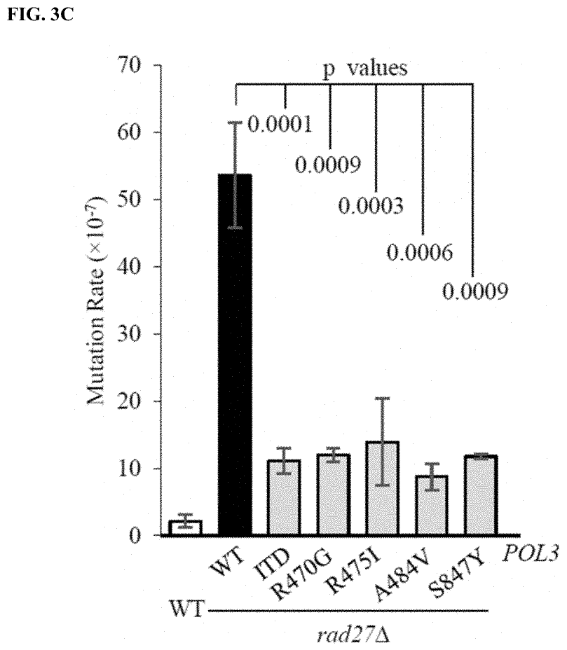

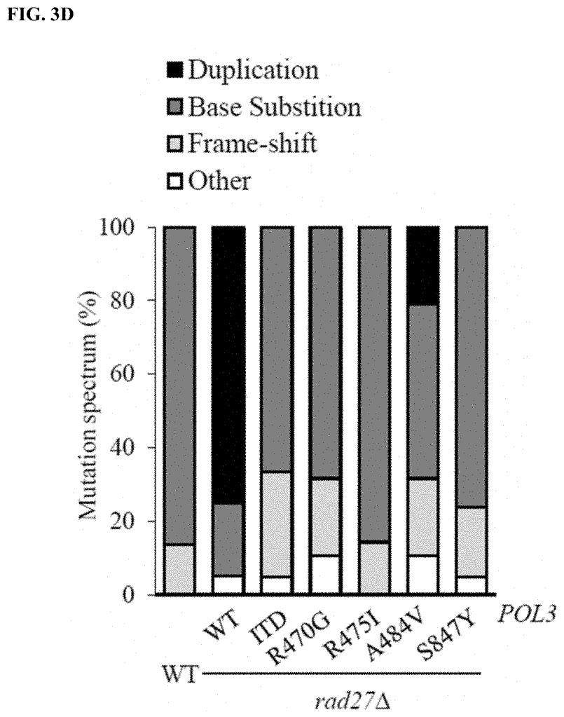

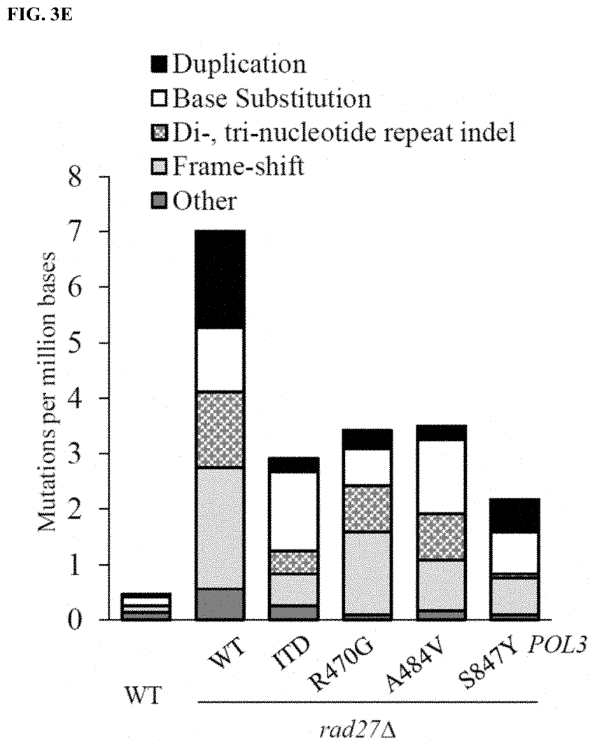

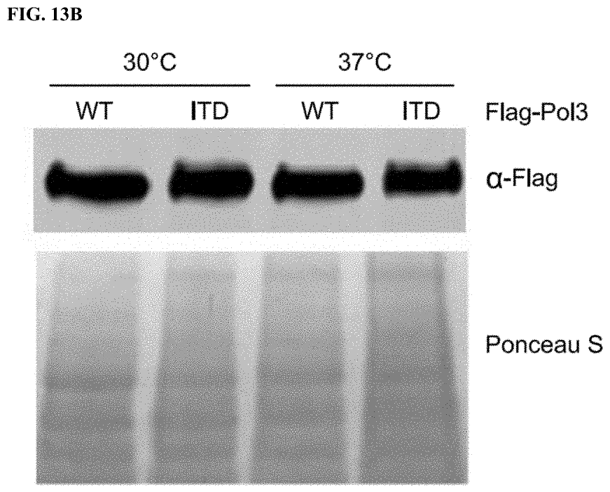

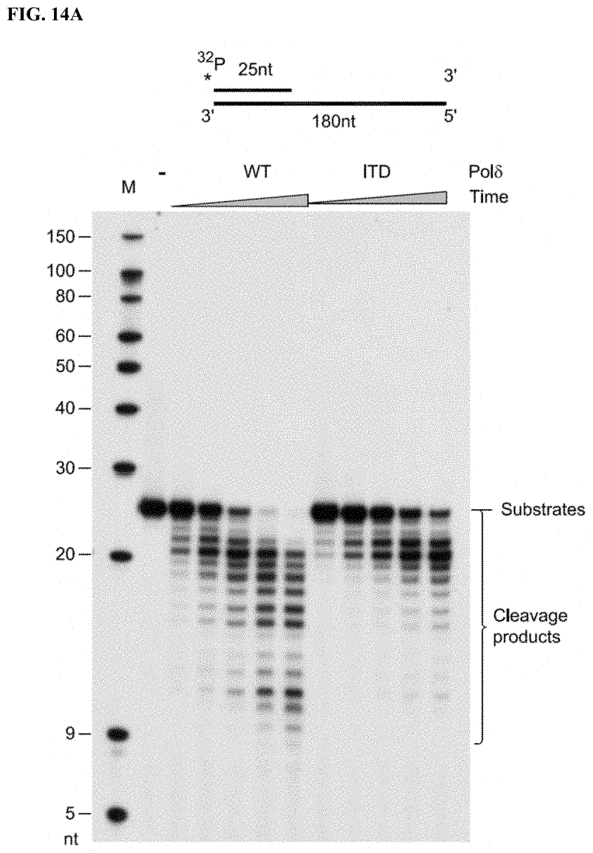

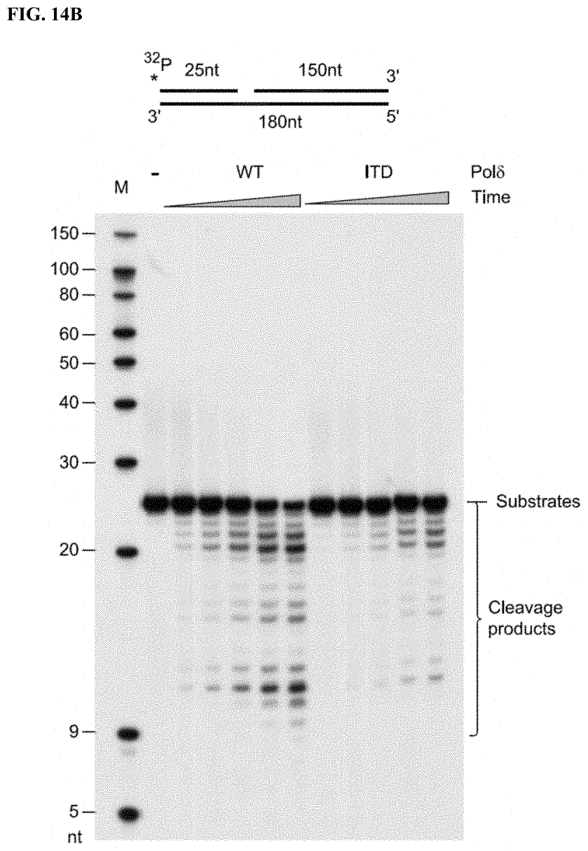

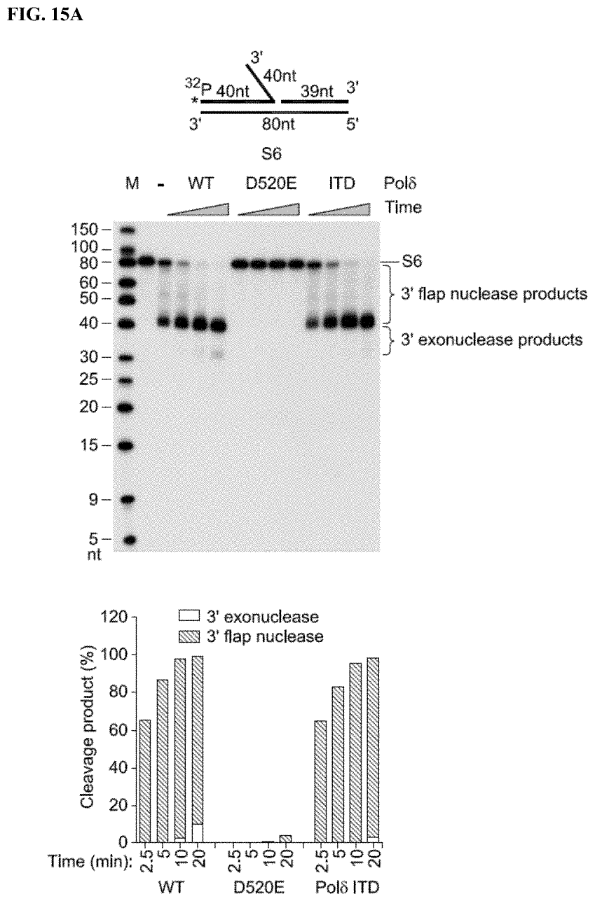

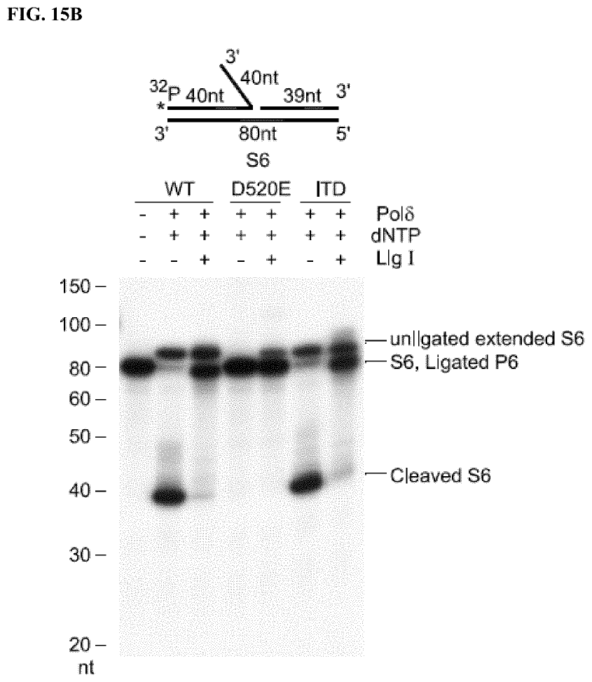

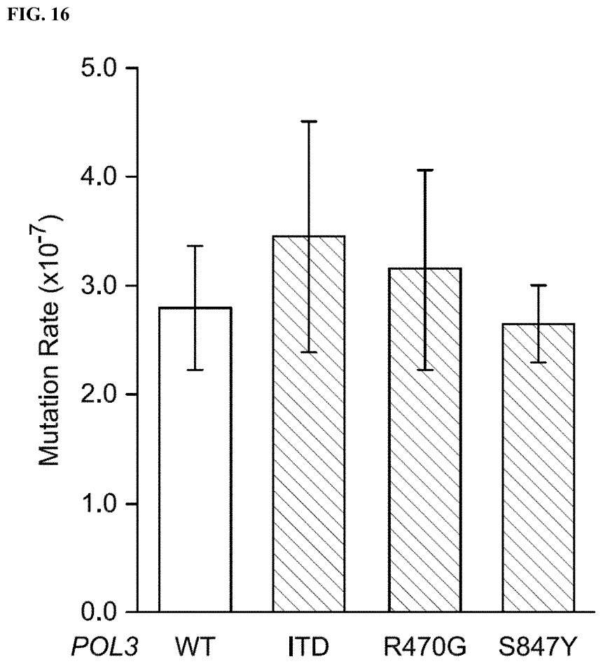

We next determined how the pol3 458-477 ITD enabled rad27Δ cells to overcome lethal stress. Because the pol3 458-477 ITD did not change Polδ protein levels in rad27Δ cells (FIG. 13), we tested if it affected biochemical properties of Polδ. We assayed the DNA polymerase and 3′ nuclease activities of a purified recombinant protein Polδ complex containing either a WT Pol3 subunit (WT Polδ) or a 458-477 ITD Pol3 subunit (hereafter called Polδ-ITD). Polδ-ITD could catalyze DNA synthesis but was less processive than WT Polδ during primer extension (FIG. 3A). Similarly, Polδ-ITD could effectively fill the gap, but it was less active than WT Polδ in displacing the downstream DNA oligo (FIG. 3B). In addition, Polδ-ITD had relatively weak 3′ exonuclease activity on DNA duplexes, compared to WT Polδ (FIG. 14). However, Polδ-ITD had similar activity to WT Polδ in cleaving the 3′ flap and generating a ligatable nick (FIG. 15). This activity likely allows cells carrying the pol3 458-477 ITD to have a similar capacity as WT cells for catalyzing 3′ flap processing for OFM. In contrast, a 3′ exonuclease-dead mutant, PolδD520E, did not cleave the 3′ flap (FIG. 15), which may explain why the PolδD520E mutation is lethal at restrictive temperature and synthetically lethal with rad27Δ (16).

We further revealed that knock-in of pol3 mutations significantly reduced the mutation rate of rad27Δ cells, as measured by Canavanine resistance (Can^{r}) (FIG. 3C) but did not affect the mutation rate of yeast cells with WT Rad27 (FIG. S12). These pol3 mutations nearly completely suppressed the occurrence of duplications (FIG. 3D). Consistent with the Can^{r} assay results, our WGS data confirmed that pol3 mutations reduced the frequency of duplications and the overall mutation frequency (FIG. 3E). Duplication mutation rate correlates with the level of 5′ flap formation (12). Thus, our biochemical and genetic results demonstrate that pol3 ITD and other point mutations can reverse the conditional lethality phenotype by limiting 5′ flap formation in rad27Δ cells.

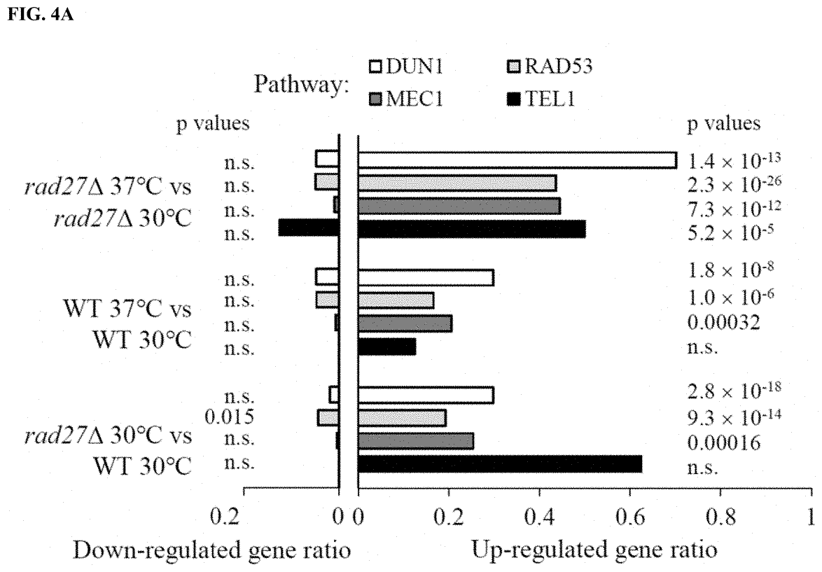

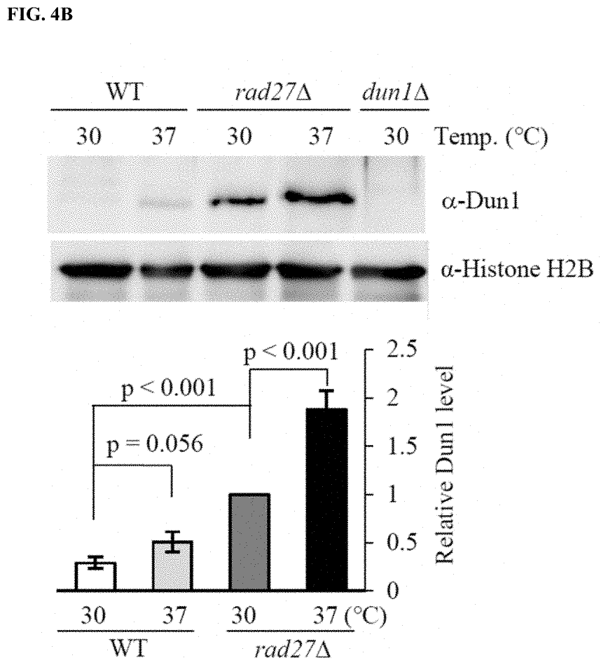

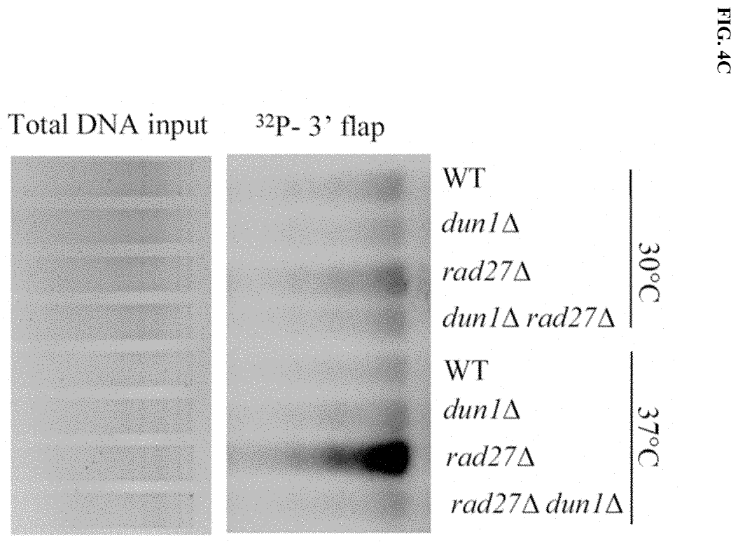

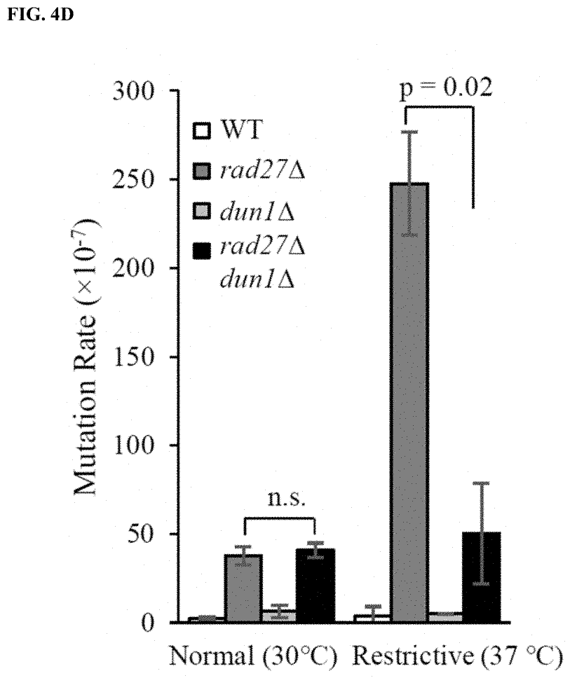

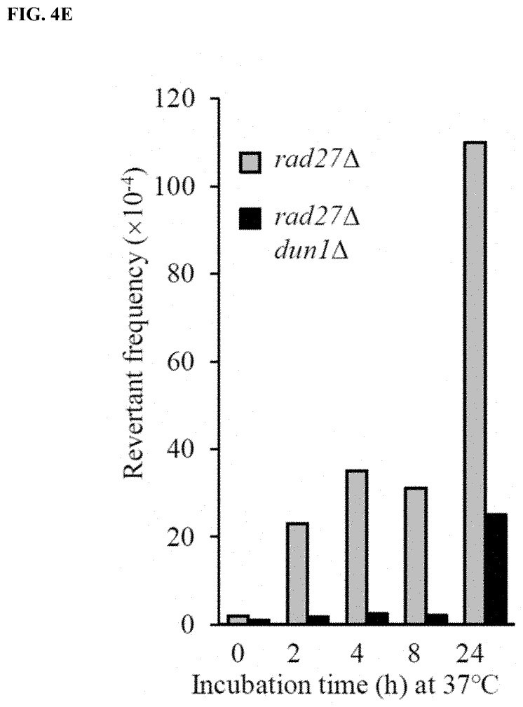

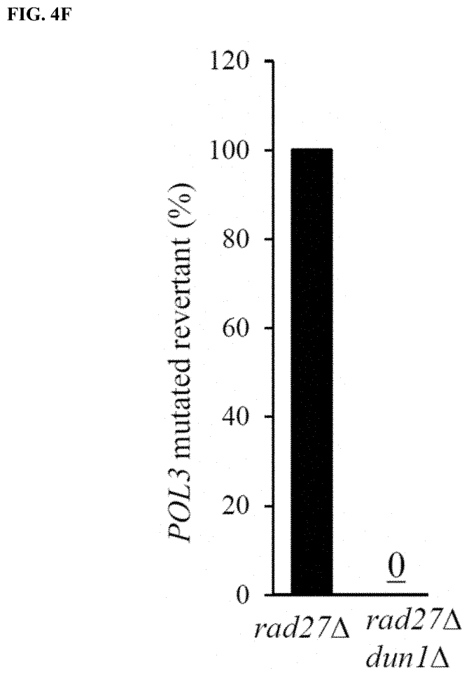

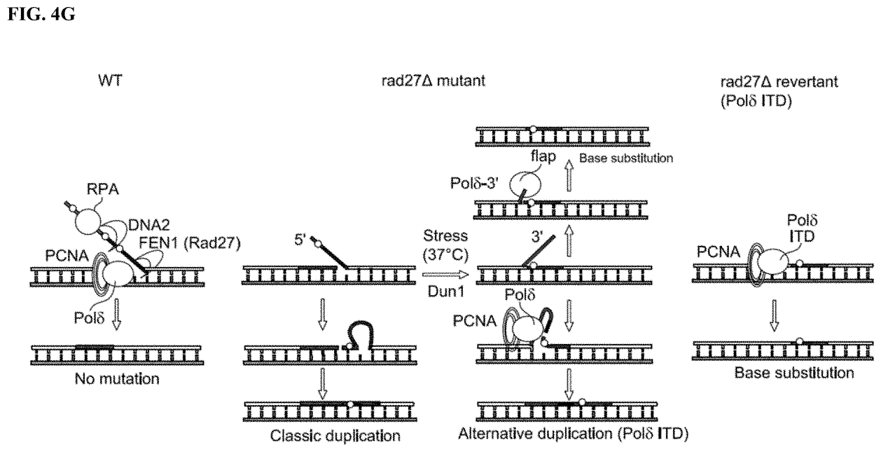

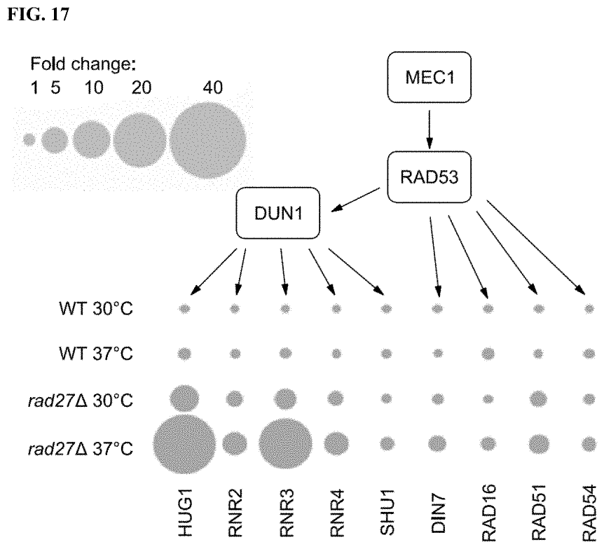

To identify the signaling pathways that induced 3′ flap-mediated OFM and led to generation of pol3 ITD, we compared the transcriptomes of WT and rad27Δ cells grown at 37° C. or 30° C. We observed that genes regulated by the checkpoint kinases Mec1, Rad53, and Dun1 were significantly up-regulated in rad27Δ cells, especially those grown at 37° C. (FIG. 4A); consistent with this, western blot analysis confirmed that chromatin-associated Dun1 protein was increased in rad27Δ cells grown at 37° C. (FIG. 4B). These results indicate activation of the Mec1-Rad53-Dun1 axis, the major signaling pathway that is activated to counteract genotoxic stress (17, 18). We further showed that downstream targets of the upregulated genes, including the stress response genes HUG1, RNR2, RNR3, and RNR4, and the DNA repair gene RAD51, were synergistically induced by rad27Δ and restrictive temperature stress (FIG. 17). RAD51 is associated with inhibition of 3′ ssDNA degradation, which at least partially explains why degradation of 3′ flaps induced by NE from rad27Δ cells grown at 37° C. was markedly less than degradation induced by WT NE (FIGS. 11A-11B).

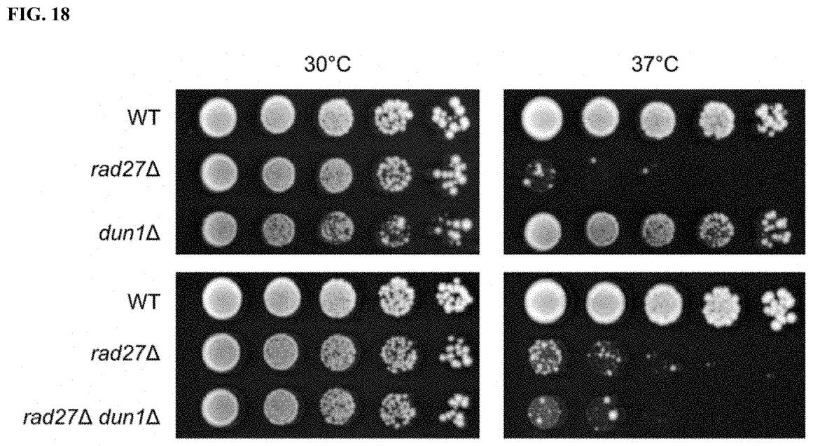

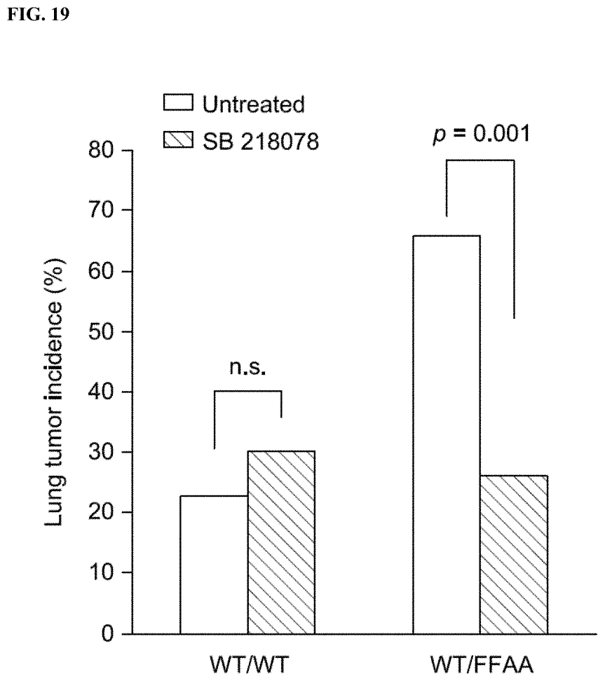

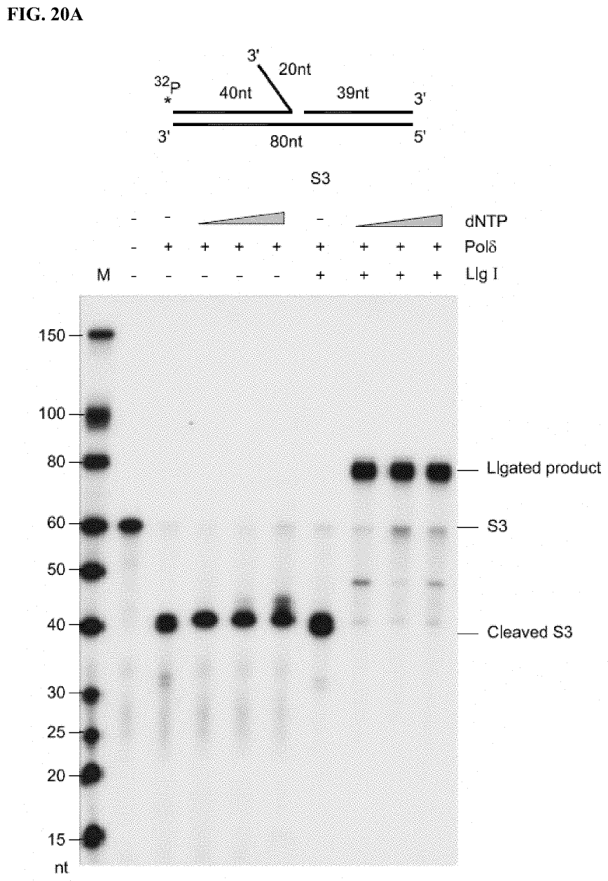

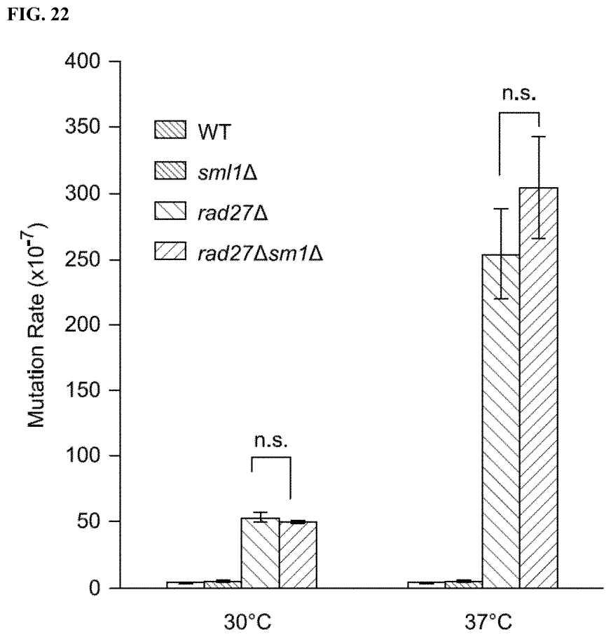

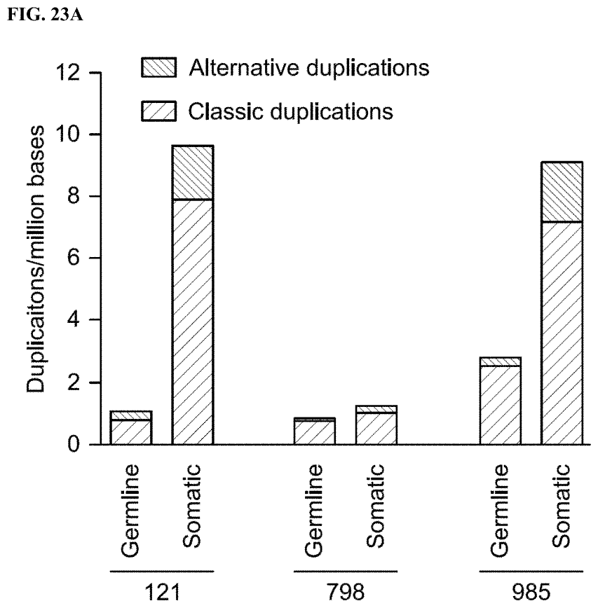

To define the role of Dun1 signaling in stress-induced mutation and generation of revertants, we deleted the DUN1 gene in WT and rad27Δ cells. We observed that knockout of DUN1 (dun1Δ) in WT or rad27Δ cells had little effect on their survival (FIG. 18), 3′ flap formation (FIG. 4C), or mutation rate at 30° C. (FIG. 4D). However, DUN1 deletion markedly reduced restrictive temperature stress-induced 3′ flap formation (FIG. 4C) and abolished restrictive temperature stress-induced mutations in rad27Δ cells (FIG. 4D). Consistent with this, DUN1 deletion inhibited generation of rad27Δ revertants (FIG. 4E, Supplementary text S6). Furthermore, all rad27Δ revertants in this experiment had pol3 mutations, predominantly the pol3 458-477 ITD, but none of the rad27Δ dun1Δ revertants had pol3 mutations (FIG. 4F). These findings indicate that Dun1 activation plays an important role in the development of restrictive temperature stress-induced mutations that can reverse the lethal phenotype of rad27Δ cells. Consistent with this finding, blocking activation of Chk1, a Dun1 functional analogue, significantly inhibited spontaneous lung cancer development in FEN1 mutant mice but not in WT mice (FIG. 19, Supplementary text S7). An important function of Dun1 activation is to induce overexpression of HUG1, RNR2, RNR3, and RNR4 for deoxyribonucleotide production. Increased deoxyribonucleotide concentrations changed the mode of action of Polδ and promoted generation of ligated extended products in vitro (FIGS. 9, 20, 21, Supplementary text S8). However, when we deleted SML1, the protein inhibitor of ribonucleotide reductase (19), to increase deoxyribonucleotide production, we did not observe increased mutation rates in rad27Δ cells (FIG. 22), indicating that an up-regulation of deoxyribonucleotide alone is not sufficient to promote alternative duplications.

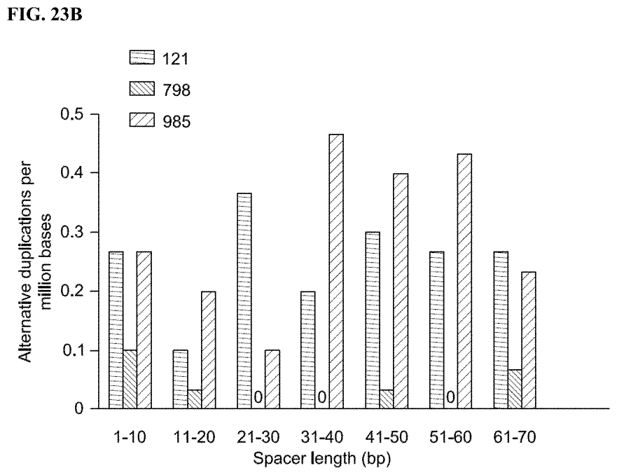

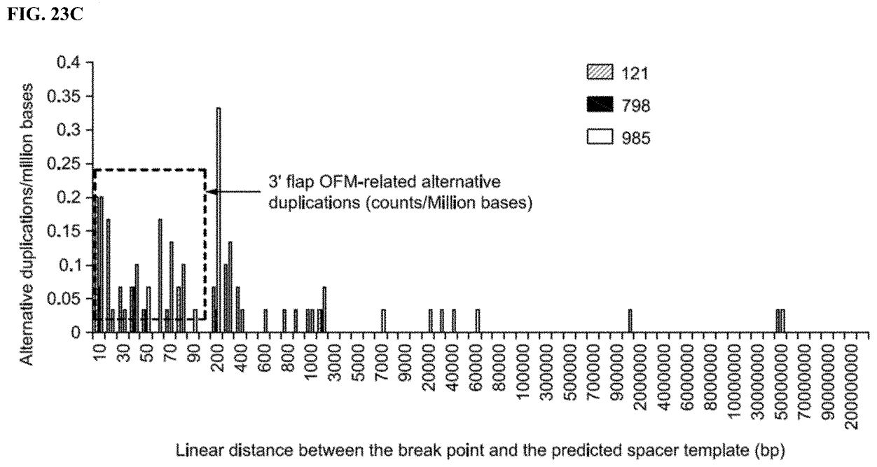

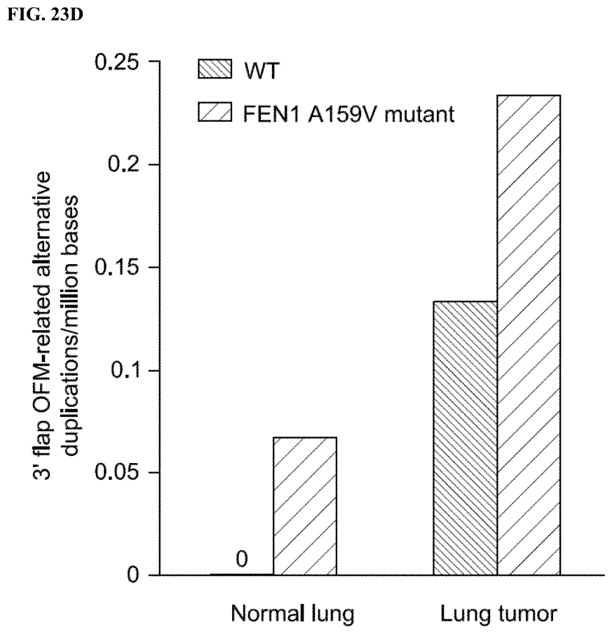

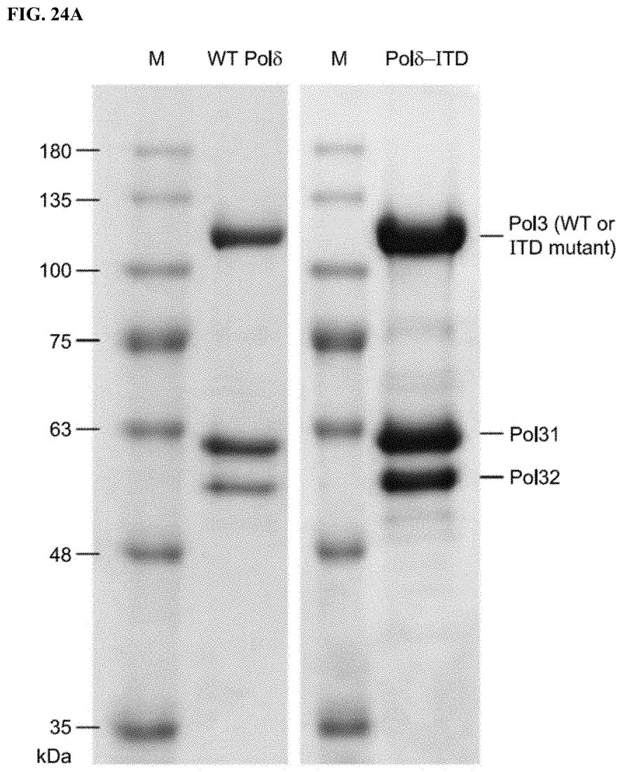

To demonstrate the relevance of stress-induced 3′ flap-based OFM and alternative duplications in rad27Δ cells to human cancers, we used whole-exome sequencing (WES) to analyze alternative duplications in human tumors and mutant mice modeling human FEN1 mutations. Alternative duplications, similar to those in rad27Δ cells grown at restrictive temperature (i.e., 3′ flap OFM-related alternative duplications), were frequent in human B cell acute lymphoblastic leukemia (FIGS. 23A-23C, Supplementary text S9). In addition, FEN1 A159V mutation, which occurs in human lung cancers (20), promoted 3′ flap OFM-related alternative duplications in mice (FIG. 23D, Supplementary text S9). Therefore, mutations in FEN1 or other OFM genes may lead to 3′ flap-based OFM, and play crucial roles for cancer cell evolution, tumor growth, and resistance.

Our current study defines error-prone processing of RNA-DNA primers during OFM (FIG. 4G). Induction of this mechanism generates alternative duplications and base substitutions. In WT cells, the displaced 5′ RNA-DNA flap is effectively cleaved by either Rad27 alone or by Dna2, which first cleaves the 5′ RNA-DNA flap in the middle, leaving a shorter 5′ DNA flap for Rad27 to subsequently cleave. When Rad27 is not available, other 5′ nucleases such as Dna2 alone or Exo1 are involved in inefficient 5′ flap processing (21, 22). Resolution of 5′ flaps also requires an alternative pathway that is mediated by the 3′ exonuclease activities of Polδ, which removes nucleotides from the 3′ end of an upstream Okazaki fragment, generating a gap for the unprocessed 5′ flap to re-anneal for ligation (16, 23). Restrictive temperature stress activates Dun1 signaling and stimulates de novo production of deoxyribonucleotides, which in turn inhibits the 3′ exonuclease activity, but not the flap nuclease activity of Polo, and induces other DNA damage responses. These molecular changes push OFM toward transformation of an unprocessed 5′ flap into a 3′ flap, either through flap equilibration (24) or the actions of helicases such as Sgs1 or Pif1, leading to a secondary structure that may result in alternative duplications, including Polδ-ITD, in revertant strains. In the revertants, Polδ mutations limit DNA displacement, thus suppressing 5′ flap formation or allowing more time for Dna2 or Exo1 to act on the 5′ flap and bypass the requirement for Rad27 (FIG. 4G).

Materials and Methods

Yeast Strains and Plasmids

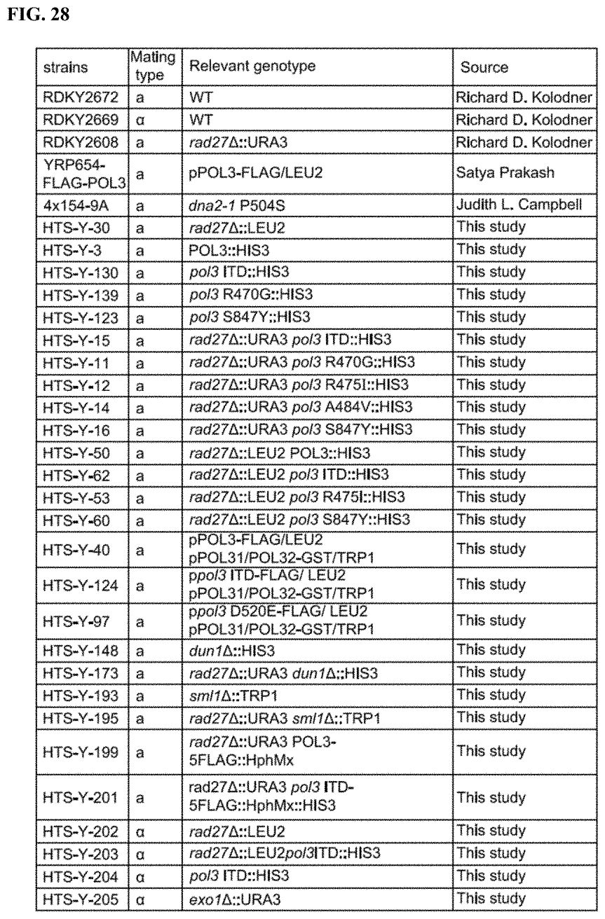

All yeast strains (see FIG. 28 for sources) used for genetic studies were derivatives of three Saccharomyces cerevisiae yeast strains: the WT control strain (RDKY2672: MATa, his3Δ200, ura3-52, leu2Δ1, trp1Δ63, ade2Δ1, ade8, hom3-10, lys2ΔRgl; RDKY2669: MATα, his3Δ200, ura3-52, leu2Δ1, trp1Δ63, ade2Δ1, ade8, hom3-10, lys2ΔBgl), the rad27Δ strain (RDKY2608: MATa, his3Δ200, ura3-52, leu2Δ1, trp1Δ63, ade2Δ1, ade8, hom3-10, lys2ΔBgl, rad27Δ::URA3) (12), and dna2-1 mutant strain, which carries the nuclease abolishing P504S dna2 mutation (4×154-9A: MATa, his3Δ200, leu2Δ1, trp1-289). All derived yeast strains are listed in FIG. 28. The genotypes of these yeast strains were verified using PCR-based genotyping. The pol3 knock-in mutant yeast strain was generated using a previously published two-step approach for knock-in of point mutations in yeast (25). A PCR-amplified DNA fragment encoding the Pol3 mutation and the selection marker HIS3 was transfected into rad27Δ yeast cells. Stably transfected cells, as indicated by growth on synthetic complete (SC)-His nutrient-deficient plates, were selected and knock-in of the pol3 mutation was confirmed by PCR-based Sanger DNA sequencing. The rad27Δ or rad27Δ pol3 ITD knock-in yeast cells were transfected with a PCR-amplified DNA fragment encoding the Flag tag and the hygromycin B selection marker. The PCR fragment was also flanked by two 39 bp fragments (Upstream: SEQ ID NO:1, Downstream: SEQ ID NO:2), corresponding to the C-terminus and 3′UTR of the POL3 gene. Flag-tagged Pol3 colonies were selected in yeast extract peptone dextrose (YPD) medium containing hygromycin B. Expression of Flag-tagged WT or ITD Pol3 in WT or rad27Δ was verified by western blot analysis using the anti-Flag tag antibody (Cat #F1804-200UG, SIGMA).

DUN1 or SML1 gene knock-out cells were constructed using a recombination-based approach. WT or rad27Δ yeast cells were transfected with a PCR-amplified DNA fragment containing the HIS3 or TRP1 gene, which was flanked by two 39 bp fragments (DUN1 upstream: SEQ ID NO:3, DUN1 downstream: SEQ ID NO:4, SML1 upstream: SEQ ID NO:5, SML1 downstream: SEQ ID NO:6), corresponding to the 5′ or the 3′ end of the DUN1 or SML1 gene, respectively, for recombination. Knock-out of DUN1 or SML1 (dun1Δ or sml1Δ) in the transformant colonies, which were selected in SC-His or SC-Trp growth medium, was verified using PCR-based Sanger DNA sequencing. The protease-deficient S. cerevisiae yeast strain YRP654 (a gift of Dr. Satya Prakash) was used to express Polo and Polo-ITD mutant complexes.

Genetic Crosses

Previous studies showed synthetic lethality of rad27Δ with exo1Δ or of rad27Δ with the nuclease activity-abolishing dna2 mutation P504S (dna2-1 mutant allele) (26, 27). To assess if double mutation of rad27Δ and pol3 ITD could rescue this synthetic lethality at 30° C., diploid yeast mutant cells were created by genetic crosses of the rad27Δ::LEU2pol3 ITD::HIS3 mutant strain with the exo1Δ::URA3 strain or with the dna2-1 strain. Ascospores from the diploid mutant cells were isolated as previously described (28, 29). The haploids, carrying either the MATa or the MATα allele, were confirmed by PCR analysis using the primers: MATa forward primer: SEQ ID NO:7, MATα forward primer: SEQ ID NO:8, and MAT reverse primer: SEQ ID NO:9. The isolated spores germinated and grew into colonies on nutrient-deficient medium plates to select spores that carried a specific combination of mutant alleles (FIGS. 26-27). In genetic crosses of the rad27Δ::LEU2pol3 ITD::HIS3 strain with the exo1A::URA3 strain, the viability of spores carrying the rad27Δ::LEU2 pol3 ITD::HIS3 and exo1A::URA3 alleles were assessed by the capacity to grow and form colonies on SC-Leu-His-Ura plates, which selected for rad27Δ pol3 ITD exo1Δ cells. In genetic crosses of the rad27Δ::LEU2pol3 ITD::HIS3 strain with the dna2-1 strain, which has no linked selection marker, the presence of viable spores carrying rad27A::LEU2pol3 ITD::HIS3 dna2-1 alleles was determined using PCR-based DNA sequencing of the DNA2 allele from independent colonies formed on SC-Leu-His plates, which selected for rad27Δ pol3 ITD cells.

Canavanine Resistance Assays

Canavanine resistance (Can^{r}) assays were used to measure mutation rates and mutation spectra. The Can^{r}-based forward mutation rate in the yeast strains was determined as previously described (12, 30). Briefly, single colonies (n=10) were picked and cultured in YPD medium overnight at 30° C. For restrictive temperature stress conditions, yeast cells were transferred into fresh medium and incubated at 37° C. for 4 h. Cells from each independent colony were then diluted into sterile water and plated onto YPD plates or plates with arginine-deficient selective medium containing 60 μg/mL canavanine (Sigma-Aldrich, C9758). Numbers of Can^{r} colonies were counted and the data were analyzed using the method of Lea and Coulson (31). In this method, the average number of Can^{r} mutations per culture M is calculated based on the equation: r0=M (1.24+ln M), in which r0 is the median number of Can^{r} colony-forming units per culture. The mutation rate is calculated using the equation r=M/N, where N is the average number of viable cells per plating. To define the mutation spectra of the yeast strains, about 20 single Can^{r} colonies were picked from canavanine plates (one colony was picked per plate) and grown in YPD medium. The total genomic DNA was extracted and purified. The CAN1 gene was PCR-amplified for each Can^{r} colony and CAN1 gene mutations were detected using Sanger sequencing.

Generation of Revertant Lines

To produce rad27Δ revertants for mutation analysis, a single rad27Δ colony was cultured in YPD medium at 30° C. The cultured cells were then diluted into fresh YPD medium and incubated at 37° C. Cells were diluted into fresh YPD medium every 4 days for 20 days. The cells were then plated onto YPD plates and incubated at 37° C. for 48 hours. Any viable colonies were considered revertants. The revertant phenotype was verified by conducting spot assays at various temperatures. Briefly, yeast cells [optical density (0D600)=about 0.4] of the indicated genetic backgrounds were serially diluted at a 1:5 ratio. The diluted cells were spotted onto YPD plates, and incubated at 30° C. (optimal temperature), 25° C. (sub-optimal temperature), or 37° C. (restrictive temperature) for 48 hours.

To evaluate the revertant frequency, rad27Δ or rad27Δ dun1Δ cells from a single colony were cultured in YPD medium overnight at 30° C. The same number of yeast cells (0.1-1 million; quantified by hemacytometer) was then cultured in fresh YPD and incubated at 37° C. for 0, 2, 4, 8, or 24 h (n=3). The cell culture was then plated onto YPD plates and incubated at 37° C. for 48 h. The number of colonies was scored and the revertant frequency (%) was calculated by dividing the number of colonies by the number of viable cells before incubation at 37° C.

MMS Sensitivity Assay

To assay the MMS sensitivity of different yeast strains, 0.005%, 0.01%, 0.015% or 0.02% MMS was added to YPD plates after autoclaving, and plates were used the same day. Yeast cells were serially diluted and spotted on YPD plates containing different levels of MMS. The plates were incubated at 30° C. for 48 h.

Animal Studies

FEN1 F343A/F344A (FFAA) heterozygous mice (WT/FFAA, 129S1 genetic background), which develop lung adenoma or adenocarcinoma at a high frequency (32), were in-line bred and housed in the Animal Resource Center at City of Hope. All experimental protocols involving animals were approved by the Institutional Animal Care and Use Committee of City of Hope in compliance with the Public Health Service policies of the United States. To determine the extent to which inhibiting Chk1 suppressed cancer development in FEN1 FFAA mutant mice, randomly selected WT or WT/FFAA male or female mice (6 months old) were treated with 0.025 mg/kg body weight SB218078 (Chk1 inhibitor) in DMSO via intraperitoneal injection once per week for four weeks. Untreated control mice were injected with the same volume of DMSO (vehicle). All mice were euthanized at 16 months of age. The presence of lung tumors was determined using anatomic and histopathological analysis in blinding fashion. For whole exome sequencing (WES) studies, WT or FEN1 A159V/WT mice (33) were euthanized at 16 months, and normal lung or lung tumors were dissected for total genomic DNA extraction and purification.

Protein Expression and Purification

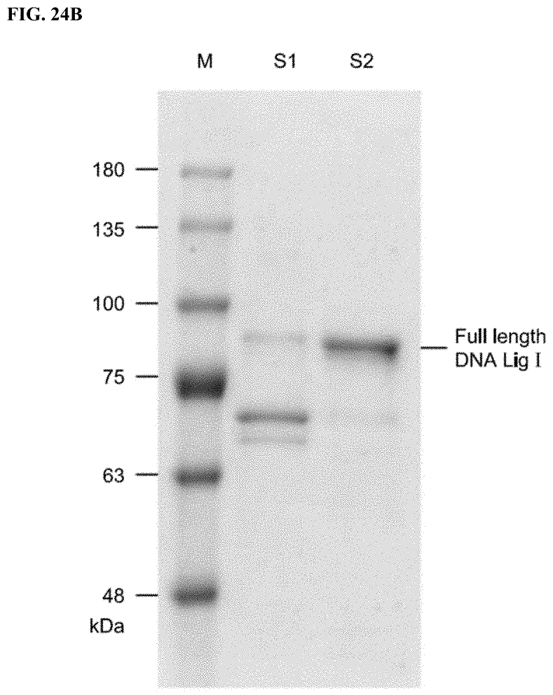

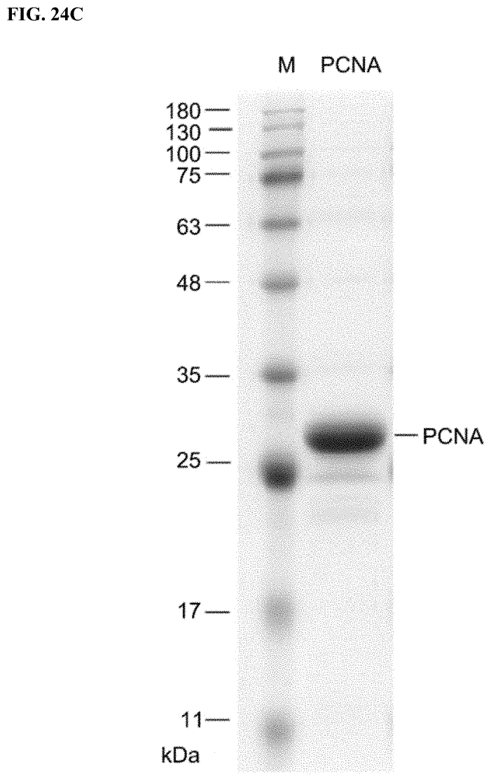

Recombinant Pol3, Pol31, and Pol32 were co-expressed in yeast cells and purification of the Polδ complex was performed as described previously (34-36). All of the following purification steps were performed at 4° C. Briefly, about 10 grams of frozen yeast cells expressing WT Polδ (Pol3, Pol31, and Pol32 subunits) or Polδ-ITD (pol3 ITD, Pol31, and Pol32 subunits) were re-suspended in 3 volumes of 1×CBB (50 mM Tris-HCl, pH 7.5, 10% sucrose, 1 mM EDTA) containing 500 mM NaCl and protease inhibitor cocktail (Thermo Scientific, 78429) and then disrupted in a Bullet Blender Gold (Next Advance, Inc) with cooling (dry ice). Cell debris was removed by centrifugation (20,000×g, 10 min), after which the clarified whole cell extract was prepared by further centrifugation (20,000×g, 120 min). Ammonium sulfate (0.28 g/mL) was added to the whole cell extract to precipitate the proteins. The resulting pellet was re-suspended in 10 mL of 1×GBB (50 mM Tris-HCl, pH 7.5, 10% glycerol, 1 mM EDTA) containing 200 mM NaCl (GBB200) and protease inhibitor cocktail and dialyzed overnight against 100 volumes of 1×GBB. The dialyzed sample was then passed slowly over a 2-mL Glutathione Sepharose 4B column (GE Healthcare, 17-0756-01), washed with 10 volumes of 1×GBB250, and equilibrated in 1×GBB150. Proteins were eluted by incubation with 1×GBB150 containing 40 mM glutathione and 0.01% Nonidet P-40 and subsequently mixed with 0.5 mL anti-FLAG M2 agarose (Sigma-Aldrich, A2220) and rocked overnight, washed with 1×GBB250, and equilibrated in 1×GBB150 containing 0.01% Nonidet P-40. The Polδ proteins were eluted by treatment with 20 units of PreScission protease (GE Healthcare, GE27-0843-01) overnight at 4° C. The eluted recombinant Polδ protein was then incubated with 100 μL of Glutathione Sepharose 4B to remove residual PreScission protease and GST-tag. The purity of the final Polδ complex was evaluated using SDS-PAGE (FIG. 24A).

Recombinant 6His-tagged yeast DNA Lig I (Cdc9) or PCNA (Pol30) was expressed in the E. coli strain BL21 (DE3) and purified using N⁺⁺-NTA agarose as previously described (37). The 6His-tagged DNA Lig I was further purified using heparin chromatography. Protein bound to the heparin column was eluted using a linear gradient of 50-500 mM KCl in buffer containing 20 mM K-PO4, pH 7.5, 0.1 mM EDTA, and 10% glycerol. The eluted peak fraction was subjected to buffer exchange with storage buffer (10 mM Tris-HCl, pH7.5, 50 mM NaCl, 10% glycerol) and concentrated using a centrifugal filter unit. The purity of the final Lig I or PCNA protein was evaluated using SDS-PAGE (FIGS. 24B-24C).

Nuclease and Polymerase Activity and Reconstitution Assays

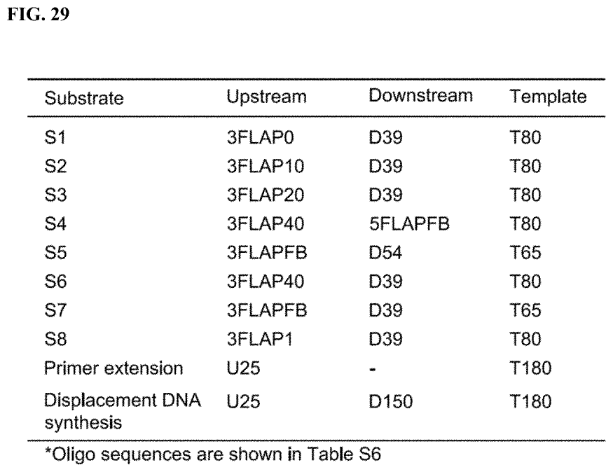

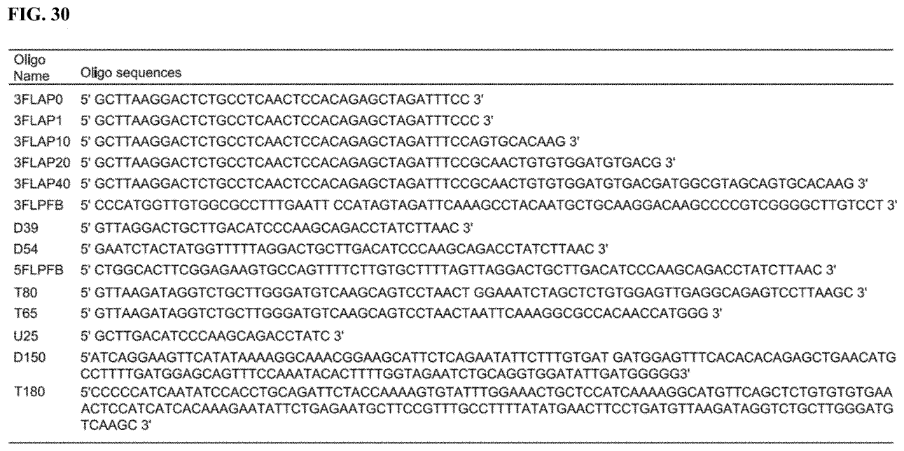

The nuclease and polymerase activities of WT yeast Polδ and Polδ-ITD were assayed using 5′ ³²P-labeled DNA substrates, which were prepared as previously described (38). The 5′ ³²P-labeled DNA substrates for primer extension or strand displacement DNA synthesis were prepared using the synthetic oligonucleotides listed in FIGS. 29-30. Purified recombinant WT Polδ or Polδ-ITD (20 nM) was incubated with yeast PCNA (100 nM) and ³²P-labeled DNA substrate (100 nM) in reaction buffer (20 mM Tris-HCl, pH 7.8, 1 mM DTT, 100 μg/mL BSA, 8 mM MgOAc2, and 1 mM spermidine) with or without each of the four deoxyribonucleotides (100 μM each) and ATP (1 mM), at 30° C. for 2.5, 5, 10, 20, or 40 min. The reactions were stopped by addition of 2× loading buffer (25 mM EDTA, 0.2% bromophenol blue, and 0.2% xylene cyanol) followed by boiling for 10 min. The reaction was resolved using 15% denaturing PAGE and visualized using radioautography.

To reconstitute the sequential reactions of 3′ flap cleavage, gap filling, and DNA ligation, a 3′ flap DNA substrate (100 nM) was incubated with Polδ (20 nM) in reaction buffer (20 mM Tris-HCl, pH 7.8, 1 mM DTT, 100 μg/mL BSA, 8 mM MgOAc2, and 1 mM spermidine) with or without each of the four deoxyribonucleotides (100 μM each) and ATP (1 mM), at 30° C. for 10 min. Purified recombinant yeast DNA Lig I (75 nM) was added to the reaction, and DNA ligation was allowed to proceed during a 10 min incubation (30° C.) or the reaction was immediately stopped by addition of 2× loading buffer to serve as an unligated control. Ligation reactions were also stopped by addition of 2× loading buffer and all samples were boiled for 10 min. Reactions were then resolved using 15% denaturing PAGE and visualized using radioautography. The 5′ ³²P-labeled 3′ flap DNA substrates S1-S8 were prepared using the synthetic oligonucleotides listed in FIGS. 29-30.

Western Blot Analysis of Chromatin-Associated Dun1 and Flag-Tagged Pol3

Chromatin-associated proteins were isolated from yeast cells as previously described (39). Briefly, harvested yeast cells were suspended in a buffer containing 0.1 M K-EDTA, pH 8.0, 10 mM DTT and incubated at 30° C. for 10 min. After centrifugation, the cell pellet was resuspended in YPD medium containing 1 M sorbitol, 10 U/mL Zymolyase (AMSBIO, 120491-1) and incubated at 37° C. for 1 h. After centrifugation, the pellet was resuspended in extraction buffer (50 mM HEPES/KOH pH7.5, 150 mM KGlu, 2.5 mM MgOAc2, 0.1 mM ZnOAc2, 1 mM DTT, 1 mM PMSF, protein inhibitor, phosphatase inhibitor, 0.25% Triton X-100) and incubated on ice for 10 min. The whole cell extract was then loaded onto extraction buffer containing 30% sucrose. After centrifugation (15,000×g, 10 min) at 4° C., the pellet (chromatin fraction) was dissolved in SDS-PAGE loading buffer and resolved using 8% SDS-PAGE. Immunoblot was conducted to analyze the chromatin-associated Dun1 using a polyclonal anti-yeast Dun1 antibody (from Wolf-Dietrich Heyer lab). Histone H2B, which was detected by immunoblot using a polyclonal anti-yeast Histone H2B (Ab18829, Abcam), was used as a chromatin marker and loading control.

To detect Flag-tagged WT or ITD Pol3, the whole cell extract was prepared and dissolved SDS-PAGE loading buffer and resolved using 8% SDS-PAGE. Immunoblot was conducted to analyze the Flag-tagged WT or ITD Pol3 using the monoclonal anti-Flag tag antibody. Ponceau S staining of total proteins was used as a loading control.

3′ flap labeling of genomic DNA

3′ flaps in yeast genomic DNA were labeled using an approach that was modified from protocols for labeling single-strand DNA (ssDNA) breaks in the genome (40, 41). High molecular weight genomic DNA from WT or rad27Δ cells grown at 30° C. with or without exposure to 37° C. (4 h) was isolated following the protocol for isolating yeast genomic DNA for detection of ssDNA breaks by sequencing (GLOE-Seq) (41). Following the protocol for nick sequencing (Nick-seq) (40), genomic DNA was fragmented by incubation with the restriction enzymes Hind III, Xba I, and Xho I, which generates 4 nt 5′ overhangs at the DNA ends. The 3′ OH at DNA nicks/gaps or at DNA ends was blocked with dideoxyribonucleotides by incubation with the 3′ exonuclease-deficient Klenow fragment (37° C., 1 h). The free 3′ OH at the 3′ flap, which was not blocked by the 3′ exonuclease-deficient Klenow fragment, was labeled with ³²P-deoxyribonucleotides by terminal DNA transferase (37° C., 1 h). The ³²P-labeled genomic DNA was denatured using 2× denaturing PAGE loading buffer, resolved using 5% denaturing PAGE, and visualized using radioautography. The total input DNA prior to ³²P labeling was resolved using gel electrophoresis (1% agarose) and visualized using SYBR green staining.

Gene Expression Profiling by RNA-Seq and Enrichment Analysis

Total RNA from WT or rad27.4 cells grown at 30° C. with or without exposure to 37° C. (4 h) was isolated using the RNeasy Mini kit (Qiagen). Two independent biological replicates were used for each group. Sequencing libraries were prepared using the TruSeq RNA Sample Prep Kit V2 (Illumina) according to the manufacturer's protocol. The cDNA libraries were prepared for sequencing using the cBot cluster generation system with the HiSeq SR Cluster Kit V4 (Illumina). Sequencing was performed in single-read mode for 51 cycles of read 1 and 7 cycles of index read using the HiSeq 2500 platform with the HiSeq SBS Kit V4 (Illumina). The real-time analysis 2.2.38 software package was used to process the image analysis and base calling. RNA-seq reads were trimmed to remove sequencing adapters by using Trimmomatic and to remove polyA tails by using FASTP. The processed reads were mapped back to the S. cerevisiae S288C genome assembly R64 (SacCer3) using STAR software v. 020201. HTSeq v.0.6.0 was applied to generate the count matrix of refSeq genes with default parameters. Differential expression analysis was conducted after normalizing the raw counts using the TMM (Trimmed Mean of M-values) normalization method in the “edgeR” package (42). Genes with a false discovery rate less than 0.05, a p value less than 0.05, and a fold-change greater than 1.5 or less than 0.67 were considered significantly up- or down-regulated, respectively. The sets of genes that were categorized as Dun1-, Mec1-, Rad53-, or Tell-dependent were retrieved from a previous study (43). The hypergeometric test (44) was used to determine if the set of up-regulated or down-regulated genes in rad27.4 or/and due to heat stress were Dun1-, Mec1-, Rad53-, or Tell-dependent.

WGS and WES Analysis

Total DNA from WT and rad27.4 yeast cells with or without knock-in of a pol3 mutation and grown at 30° C. or 37° C. was isolated using the YeaStar Genomic DNA kit (ZYMO Research, D2002). For sequencing library preparation, 40 ng of yeast genomic DNA from each sample was fragmented using a Covaris LE220-plus with a peak size setting of 250 bp. The libraries were prepared using the Kapa DNA HyperPrep Kit (Kapa, Cat KK 8700) according to the manufacturer's protocol with 4-cycles of PCR. Libraries were sequenced on an Illumina HiSeq2500 using a paired end mode of 2×101 cycles. Two independent biological replicates were used for each group. Reads were filtered using Cutadapt v1.18 to remove low-quality reads and sequencing adapters and were aligned to the SacCer3 reference genome using NovoAlign v3.02.07. Only reads that aligned to unique genome locations were kept for variant calling. Samtools v1.10 and VarScan v.2.3.9 (45) were used to identify somatic single nucleotide variants and small indels. Structural variants such as classic or alternative duplications were detected by Pindel (46) using default parameters. To calculate the mutation frequency, germline mutations that were detected in both WT and rad27.4 or in both rad27Δ and rad27Δ pol3 knock-in double mutants were filtered out, and the counts of each type of mutation were divided by the number of base pairs in the yeast genome.

Duplications in normal lung and lung tumors from WT or A159V/WT mice and in human B cell acute lymphoblastic leukemia (ALL) were analyzed in WES data using the Pindel program (46). WES data from three human B cell ALL patients (#121, #798, and #985) were randomly selected from a published WES dataset of paired tumor vs. normal tissues (peripheral blood samples collected after complete remission) (47). WES was conducted on genomic DNA isolated from normal lung and lung tumors from WT or A159V/WT mice as previously described (48). Duplications were scored if at least three supporting tracks were detected. The intervening spacer DNA sequences detected in the mouse or human specimens were mapped to the mouse or human genome, respectively, using the BLAT function of the UCSC genome browser (49). The frequencies of the duplications were estimated by dividing the number of duplications by the size of the mouse exome (about 30 million base pairs) or human exome (˜30 million base pairs).

Statistical Analysis

Two-tailed student's t-test was used to determine significance in differences in mutation rate assays and the hypergeometric test was used to determine the significance in the enrichment of a signaling pathway. Fisher exact test was used to determine the significance of cancer incidence between the treated and untreated groups.

Supplementary Text

Supplementary text S1. Mapping the Pol3 mutations detected in the revertants to the functional domains of Pol3

An in-frame internal tandem duplication ([ITD]; 458-477) and four point mutations (R470G, R4751, A484V, and H495Q) occurred within the POL3 nuclease domain near the Exo II (Nx3F/YD), Exo III (Yx3D), and Pol IV motifs (FIG. 1B). We also identified an in-frame ITD (591-598) within the Pol II motif, an S847Y mutation within the Pol V motif, and a P965T mutation in motif 3 of the C-terminal domain (CT3) (FIG. 1B).

Supplementary text S2. Genetic crosses of the rad27Δ pol3 ITD strain with the exo1Δ or the dna2-1 strain.

rad27Δ cells display synthetic lethality with exo1 or dna2 deficiency (26, 27). To determine if pol3 ITD could suppress such lethality, we conducted genetic crosses to create spores carrying rad27Δ pol3 ITD exo1Δ or rad27Δ pol3 ITD dna2-1 triple-mutant alleles. In the cross between rad27Δ::LEU2 pol3 ITD::HIS3 and exo1Δ::URA3 strains, 2788 colonies grew on YPD plates, but only 18 colonies grew on SC-Leu-His-Ura selection plates (FIG. 26). The viable spore ratio (0.006) of rad27Δ pol3 ITD exo1Δ was markedly less than the expected spore ratio (0.125), indicating the triple-mutant was lethal. Similarly, in the control cross between rad27Δ::LEU2 and exo1Δ::URA3 strains, 2865 colonies grew on YPD plates, but only 26 colonies grew on SC-Leu-Ura selection plates (FIG. 26). The viable spore ratio (0.009) of rad27Δ exo1Δ was markedly less than the expected spore ratio (0.25), indicating the double-mutant was lethal. This is consistent with a previous study showing that rad27Δ exo1Δ cells are inviable (27). In contrast, in the cross between pol3 ITD::HIS3 and exo1Δ::URA3 strains, 2288 and 610 colonies grew in the YPD and SC-His-Ura plates, respectively (FIG. 26). The viable spore ratio (0.27) of pol3 ITD exo1Δ was close to the expected pol3 ITD exo1Δ spore ratio (0.25), indicating that pol3 ITD in combination with exo1Δ results in viable spores.

The dna2-1 allele (P504S) has no selection marker. Therefore, we used PCR-based DNA sequencing to genotype the DNA2 mutation in the yeast cells. In the cross between rad27Δ::LEU2 pol3 ITD::HIS3 and dna2-1 strains, rad27Δ pol3 ITD cells grown on the SC-Leu-His plate were genotyped. Of 60 independent rad27Δ pol3 ITD colonies sequenced, no dna2-1 allele was detected (FIG. 27). In the control cross between rad27Δ::LEU2 and dna2-1 strains, no dna2-1 allele was detected in 40 independent rad27Δ colonies (FIG. 27). This is consistent with a previous study showing that rad27Δ dna2-1 cells are inviable (26), However, 18 out of 38 independent colonies carried the dna2-1 allele from the cross between pol3 ITD::HIS3 and dna2-1 strains (FIG. 27). The viable spore ratio (0.47) agreed with the expected pol3 ITD dna2-1 ratio (0.5), indicating that pol3 ITD in combination with dna2-1 results in viable spores.

Supplementary text S3. Three types of hairpin-forming alternative duplications in rad27Δ cells grown at 37° C.

We observed three types of alternative duplications based on hairpin structure-forming sequence features. In type 1 alternative duplications, the 5′ DNA sequence of the downstream duplication unit could form a 5′ fold-back structure (FIGS. 2D, 8A), while the 3′ end of the upstream duplication unit was complementary to nearby downstream DNA sequences (yellow highlighted segment, FIG. 8A), allowing potential invasion and extension into the downstream DNA duplex. The DNA sequence of the spacer (green segment) corresponded to the predicted extended DNA sequence of the 3′ end of the upstream duplication unit (FIGS. 2D, 8A-8B). In type 2 alternative duplications, the 3′ sequence of the duplication unit could form a 3′ fold-back structure and the predicted extension of this 3′ fold-back corresponded to the spacer (green segment, FIGS. 8A, 8C). The 5′ sequence of the downstream duplication unit was complementary to the nearby upstream DNA sequences (yellow highlighted segment, FIG. 8A), allowing strand exchange and producing a ligatable nick (FIGS. 2D, 8A, 8C). Similar to the type 2 alternative duplications, type 3 duplications also had a 3′ fold-back structure-forming DNA sequence and the spacer corresponded to the predicted extended DNA sequence of the 3′ fold-back (FIGS. 2D, FIG. 8A, 8D). However, the 3′ end of the spacer was complementary to the DNA sequence on the template strand near the 5′ end of the upstream duplication unit (yellow highlighted segment, FIG. 8A). This sequence feature allowed strand exchange to generate a ligatable DNA nick (FIG. 2D, 8D).

Supplementary Text S4. Reconstitution of 3′ Flap-Based OFM

To define 3′ flap-based OFM and the formation of alternative duplications, we reconstituted the sequential reactions of 3′ flap cleavage, DNA synthesis, and ligation of oligo-based DNA substrates with 3′ flaps of 10 or 20 nt to represent 3′ flaps that were converted from short 5′ flaps (S2 and S3, FIG. 9B). We found that PolO could effectively cleave a 3′ flap of 10 or 20 nt and stop at the junction of the 3′ flap and DNA duplex in the presence of deoxyribonucleotides, generating ligatable DNA nicks for DNA Lig I (FIGS. 2H, 9). The ssDNA binding protein RPA did not block Polδ-mediated 3′ flap cleavage or subsequent nick ligation (FIG. 2H). To determine if formation of a hairpin structure from the 3′ flap could lead to alternative duplications, we reconstituted 3′ flap-based OFM using the DNA substrates S4 and S5, which resemble type I or type II alternative duplication, respectively (FIG. 2I). In the absence of deoxyribonucleotides, Polδ cleaved the 3′ flap in S4, producing a gapped DNA duplex (FIG. 2I). However, in the presence of deoxyribonucleotides, an extended unligated product of about 85 nt was produced, which was ligated with the fold-back 5′ flap by Lig I to form a ligated extended product (˜160 nt, FIG. 2I). We found that the 3′ flap fold-back structure in S5 was resistant to cleavage by the 3′ nuclease activity of Polδ even in the absence of deoxyribonucleotides. Addition of deoxyribonucleotides to the reaction led to extension of the 3′ flap fold-back into a about 95 nt product, which was ligated into a product of about 160 nt (FIG. 2I). RPA slightly enhanced formation of the ligated extended products (FIG. 2I). These data suggest that 3′ flap-based OFM may result in alternative duplications, including mutations with features similar to pol3 458-477 ITD. However, we also found that when extension of the annealed 3′ flap could not generate ligatable nicks, only unligated extended products were produced (FIGS. 10A-10D), leading to failure of 3′ flap-based OFM. The reconstitution assays showed that the 3′ nuclease activities of Polθ and Lig I are sufficient to complete 3′ flap processing for OFM.

Supplementary text S5. 3′ flap cleavage patterns by recombinant Polδ and NE

The 3′ flap cleavage pattern produced using purified recombinant Polδ for a simple 3′ flap was similar to that produced by NE from WT cells (FIG. 11A). Both could remove a 3′ flap at the ssDNA-dsDNA junction. However, purified recombinant Polδ but not NE further effectively cleaved the intermediate DNA nick, producing DNA gaps. Purified Polδ failed to cleave hairpin-forming 3′ flaps, but NE from WT cells effectively cleaved hairpin-forming 3′ flaps around the dsDNA-ssDNA junction (FIG. 11B). However, NE from rad27Δ cells, especially those grown at 30° C., showed greatly reduced activity in processing simple or hairpin-forming 3′ flaps (FIGS. 11A-11B).

Supplementary Text S6. Revertant Frequency in Rad27Δ and Rad27Δ Dun1Δ Cells

To determine the impact of Dun1 signaling on the generation of rad27Δrevertants, we measured the revertant frequency in rad27Δ and rad27Δ dun1Δ cells following increasing periods of exposure to the restrictive temperature (37° C.). Prior to incubation at 37° C., both rad27Δ and rad27Δ dun1Δ cells had similarly low revertant frequencies (about 0.02%, FIG. 4E). Incubation of rad27Δ cells at 37° C. for 2-8 h led to a more than 15-fold increase in the revertant frequency to about 0.3%. In contrast, incubation of rad27Δ dun1Δ cells at 37° C. for 2-8 h had little effect on the revertant frequency (FIG. 4E). Both rad27Δ and rad27Δ dun1Δ cells exposed to heat stress for 24 h showed increased revertant frequencies of about 1.0% or about 0.2%, respectively (FIG. 4E). This marked increase in revertant frequency at 24 h was likely mainly due to amplification of selected revertants rather than the generation of new revertants.

Supplementary Text S7. Impact of Chk1 Inhibition on Cancer Development in FEN1 Mutant Mice

Functional deficiency due to FEN1 (the mammalian homolog of Rad27) mutations promotes DNA mutations and chromosome arrangements, leading to a high frequency of cancer in mice (50). This suggests that FEN1 mutant tumor-initiating cells can overcome OFM defects and replication stress. We previously observed that the FEN1 F343A/F344A (FFAA) mutation, which disrupts the FEN1/PCNA interaction and recruitment of FEN1 to replication forks, resulted in unligated Okazaki fragments and activation of the Chk1 signaling pathway (32). Heterozygous FFAA mutant mice develop lung adenoma and adenocarcinoma at high frequencies (32). To determine if cancer development in FEN1 FFAA mutant mice depends on activation of the Dun1 functional analogue, Chk1, we treated WT and FFAA mice with the Chk1 inhibitor SB218078 (51). This treatment significantly inhibited spontaneous lung cancer development in WT/FFAA mice but not in WT mice (FIG. 19). These results suggest that suppression of stress-induced DNA damage response signaling is an effective approach for chemoprevention and may provide a new way to inhibit drug resistance.

Supplementary Text S8. Impact of Deoxyribonucleotides on 3′ Flap Processing and Mutations

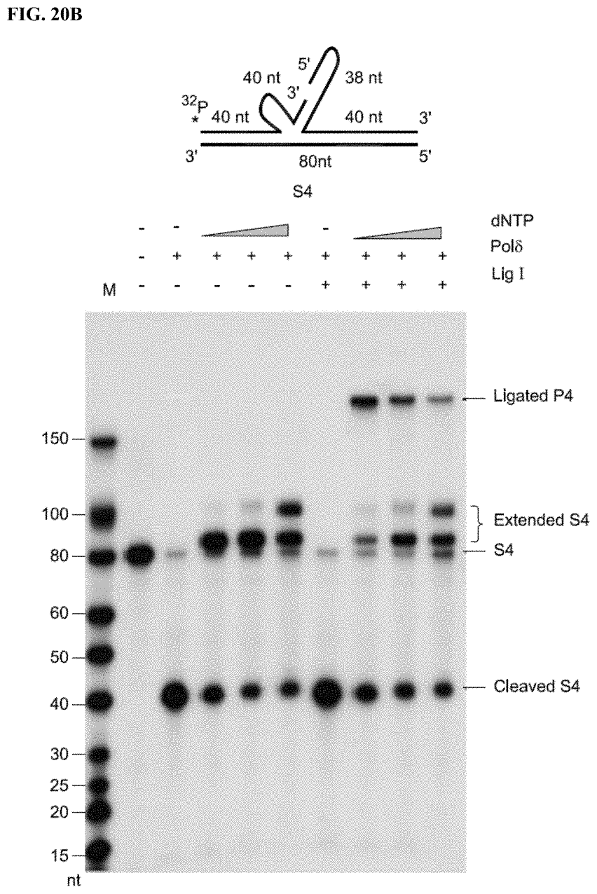

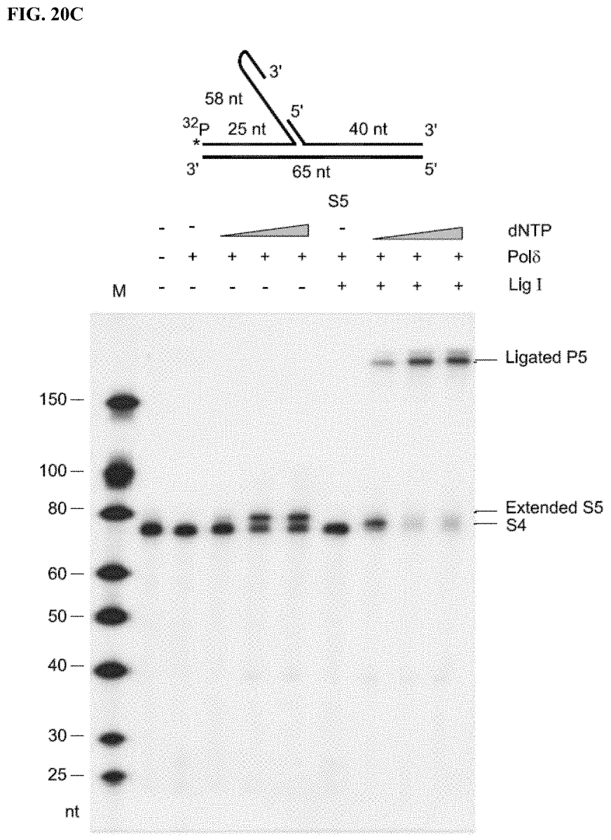

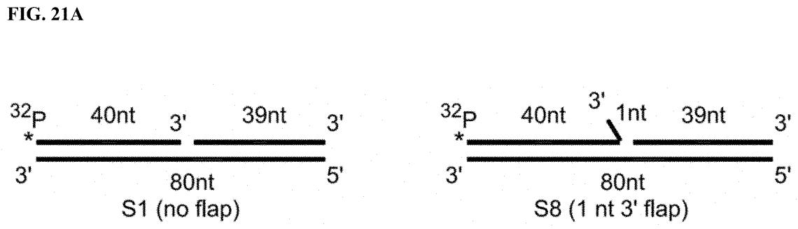

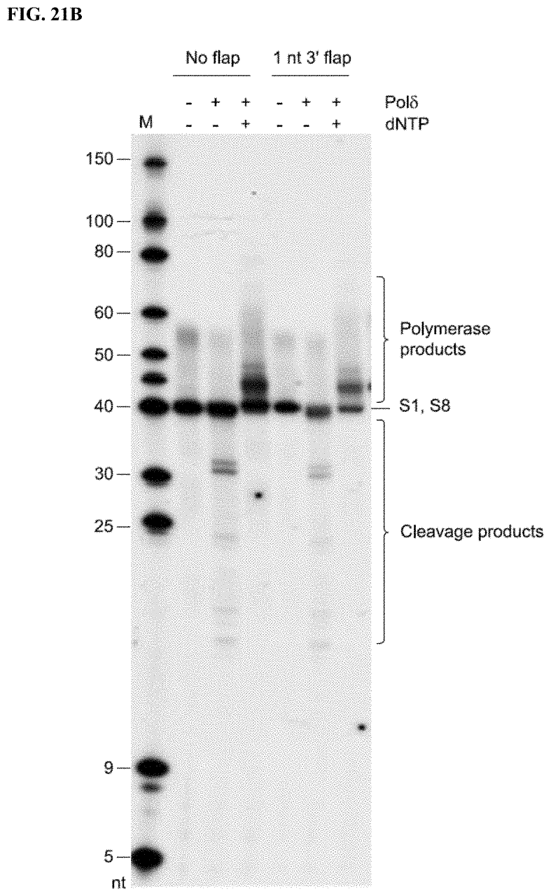

Restrictive temperature stress activates Dun1, which up-regulates expression of HUG1, RNR2, RNR3, and RNR4 for de novo production of deoxyribonucleotides. We determined that impact of deoxyribonucleotides on processing of 3′ flaps by Polδ in vitro. In the absence of deoxyribonucleotides, Pol0 effectively cleaved the 3′ flap as well as intermediate DNA nicks (FIG. 9). Deoxyribonucleotides did not affect Polδ 3′ flap cleavage activity but did inhibit the 3′ exonuclease activity of Pol0 to cleave the intermediate DNA nick, and switched the function of Polδ to gap-filling DNA synthesis (FIG. 9). Increasing the deoxyribonucleotide concentration did not affect 3′ flap processing but promoted 3′ flap extension and increased levels of unligated and ligated extended products in reconstitution assays (FIGS. 20A-20C). In addition, it has been suggested that the 3′ exonuclease activity of Polδ could degrade the upstream Okazaki fragment from the 3′ end to create a gap (16, 23). The 5′ flap could re-anneal into the gap and be resolved if this process occurred during OFM. However, we discovered that in the presence of deoxyribonucleotides, creation of such a gap by the 3′ exonuclease activity of Polδ on a nicked DNA substrate with no 3′ flap or with a 1 nt 3′ flap, representing 3′ end fraying (23), was greatly inhibited (FIG. 21). Thus, an increase in deoxyribonucleotide production due to Dun1 activation suppresses gap formation by the 3′ exonuclease activity of Polδ and pushes the process toward 3′ flap-based OFM and generation of pol3 458-477 ITD, which leads to revertant development. We further tested whether up-regulation of deoxyribonucleotides promotes DNA mutations in vivo. We found that SML1 deletion, which results in increased dNTP pools but does not affect other Dun1-mediated cellular processes (19), did not increase mutation rate in either WT or rad27Δ cells (FIG. 22), suggesting that up-regulation of dNTP levels alone is not sufficient to promote mutations.

Supplementary text S9. 3′ flap OFM-related alternative duplications in mouse and human cancers.

We sought to determine the human cancer relevance of 3′ flap-based OFM and the related alternative duplications. We noticed that the alternative duplications observed in rad27Δ cells grown at 30° C. consisted of a duplication unit (less than 100 bp in length) and a short intervening spacer sequence that could be mapped to the nearby DNA sequence (usually no farther than 100 bp). Such alternative duplications may be produced through the 3′ flap-based OFM pathway, therefore we defined them as 3′ flap OFM-related alternative duplications. To evaluate the status of these duplications in human cancers, we re-analyzed a published WES dataset of paired tumor vs. normal tissue (peripheral blood samples collected during complete remission) (47). WES data from three randomly selected B cell ALL patients were analyzed using the Pindel program to define the duplications in these cancer specimens. We found that the frequencies of germline alternative duplications in the peripheral blood of patients #121 and #985 were relatively low, but the frequencies of somatic alternative duplications in the cancer samples were remarkably higher than the corresponding germline duplications (FIG. 23A). Frequencies of both the germline and somatic alternative duplications in patient #798 were relatively low (FIG. 23A). The lengths of spacers in the alternative duplications in the three ALL specimens ranged from 1 nt to 70 nt (FIG. 23B). This was similar to the spacer lengths we observed for stress temperature-induced alternative duplications in rad27Δ yeast (FIG. 2B). To further define the origin of these spacer sequences and evaluate the frequency of 3′ flap OFM-related alternative duplications, we mapped the spacer DNA sequences across the human genome. We detected 0.6, 0.2, and 0.9 3′ flap OFM-related alternative duplications per million bases in patients #121, #798, and #985 respectively (FIG. 23C).

FEN1 mutations have been detected in human cancers and functional deficiency in FEN1 has been linked to cancer initiation and progression and to development of resistance to cancer therapies (20, 33, 50). Therefore, FEN1 mutations in human cancers may lead to 3′ flap-based OFM, and thus play crucial roles in cancer cell evolution, tumor growth, and resistance to cancer therapy. To determine if FEN1 mutations had a positive correlation with 3′ flap OFM-related alternative duplications, we conducted WES on normal lung and lung tumor tissues from WT and FEN1 A159V/WT mice, which are an appropriate model for human lung cancer patients carrying the A159V FEN1 mutation (20,33). We observed that normal lung tissues from the A159V/WT mouse but not normal lung tissues from the WT mouse had somatic 3′ flap OFM-related alternative duplications (FIG. 23D). We also found that both A159V/WT and WT lung tumor tissues had considerably more 3′ flap OFM-related alternative duplications than the corresponding normal lung tissues, but that the alternative duplication frequency in the A159V/WT lung tumors was considerably higher than in the WT tumors (FIG. 23D). These findings indicate that FEN1 deficiency promotes 3′ flap OFM-related alternative duplications, which have a positive correlation with cancer development, even in the WT FEN1 genetic background.

### Example 2

Protocol for Detection of 3′ Flap in Genomic DNA

Extract high molecular weight genomic DNA.

High molecular weight genomic DNA from yeast and human cells were purified using the AMPure XP beads according to the supplier's instruction.

Genomic DNA fragmentation.

Purified genomic DNA was fragmented by incubation with the restriction enzymes Hind III, Xba I, and Xho I, which generates 4 nt 5′ overhangs at the DNA ends. Briefly, 50 units of each of the restriction enzyme (NEB) was mixed with 3 ug genomic DNA in the Cutsmart reaction buffer (NEB) (final volume 100 ul). The reaction was conducted at 37° C. overnight. The restriction enzymes were inactivated by incubation the reaction at 72° C. for 10 minutes

Blocking Free′ 3′ OH Groups at Nick and DSB Ends

To block the 3′ OH at DNA nicks/gaps or at DNA ends, the fragmented genomic DNA (1.5 ug) was incubated with 1 mM dideoxyribonucleotides and 3′ exonuclease-deficient Klenow fragment (50 units) at 37° C. for 1 h. The DNA fragments were purified by Biospin 6 column (BioRad)

3′ Flap Labeling and Visualization

The free 3′ OH at the 3′ flap, which was not blocked by the 3′ exonuclease-deficient Klenow fragment, was labeled by incubation of the end/nick blocked genomic DNA sample (300 ng) with ³²P-deoxyribonucleotides (5 uci) and terminal DNA transferase (20 units) at 37° C. for 1 h. The ³²P-labeled genomic DNA was denatured using 2× denaturing PAGE loading buffer, resolved using 5% denaturing PAGE, and visualized using radioautography. The total input DNA prior to ³²P labeling was resolved using gel electrophoresis (1% agarose) and visualized using SYBR green staining.

Informal Sequence Listing

SEQ ID NO: 1 =
5′ AAAGAGCTGCAGGAGAAAGTAGAACAATTAAGCAAATGG
SEQ ID NO: 2 =
5′ TATCTATTTATATATACATATATATCCACCAACATGCAA
SEQ ID NO: 3 =
5′ TAGTCGAGAGTAACAAGTAAAGGGGCTTAACATACAGTA
SEQ ID NO: 4 =
5′ TGCATGTTGGTGGATATATATGTATATATAAATAGATAC
SEQ ID NO: 5 =
5′ CTCACTAACCTCTCTTCAACTGCTCAATAATTTCCCGCT
SEQ ID NO: 6 =
5′ GGAAATGGAAAGAGAAAAGAAAAGAGTATGAAAGGAACT
SEQ ID NO: 7 =
5′ ACTCCACTTCAAGTAAGAGTTTG 3′
SEQ ID NO: 8 =
5′ GCACGGAATATGGGACTACTTCG 3′
SEQ ID NO: 9 =
5′ AGTCACATCAAGATCGTTTATGG 3′

### REFERENCES

1. D. M. Fitzgerald, et al, Annu. Rev. Cancer Biol. 1, 119-140 (2017).
2. R. S. Galhardo, et al, Crit. Rev. Biochem. Mol. Biol. 42, 399-435 (2007).
3. T. Ogawa, et al, Annu. Rev. Biochem. 49, 421-457 (1980).
4. P. M. J. Burgers, et al, Annu. Rev. Biochem. 86, 417438 (2017).
5. S. A. Nick McElhinny, et al, Mol. Cell 30, 137-144 (2008).
6. S. Waga, et al, Nature. 369, 207-212 (1994).
7. M. R. Lieber, et al, BioEssays 19, 233-240 (1997).
8. S. H. Bae, et al, Nature 412, 456-461 (2001).
9. Y. Liu, et al, Annu. Rev. Biochem. 73, 589-615 (2004).
10. M. S. Reagan, et al, J. Bacteriol 177, 364-371 (1995).
11. K. C. Sitney, et al, Cell 56, 599-605 (1989).
12. D. X. Tishkoff, et al, Cell 88, 253-263 (1997).
13. A. Mariño-Enriquez, et al, Am. J. Surg. Pathol. 42, 335-341 (2018).
14. P. D. Kottaridis, Blood 98, 1752-1759 (2001).
15. H. Kiyoi, Leukemia 12, 1333-1337 (1998).
16. Y. H. Jin, J. Biol. Chem. 278, 1626-1633 (2003).
17. Z. Zhou, Cell 75, 1119-1127 (1993).
18. X. Zhao, Proc. Natl. Acad. Sci. U.S.A 99, 3746-3751 (2002).
19. A. Chabes, J. Biol. Chem. 274, 36679-36683 (1999).
20. L. Zheng, H. Dai, M. Zhou, M. Li, P. Singh, J. Qiu, W. Tsark, Q. Huang, K. Kernstine, X. Zhang, D. Lin, B. Shen, Fen1 mutations result in autoimmunity, chronic inflammation and cancers. Nat. Med. 13, 812-819 (2007).
21. S. H. Bae, J. Biol. Chem. 275, 38022-38031 (2000).
22. P. T. Tran, DNA Repair (Amst) 1, 895-912 (2002).
23. Y. H. Jin, Proc. Natl. Acad. Sci. U.S.A 98, 5122-5127 (2001).
24. Y. Liu, Mol. Cell Biol. 24, 4049-4064 (2004).
25. A. Toulmay, Yeast (Chichester, England) 23, 825-831 (2006).
26. M. E. Budd, Mol. Cell. Biol. 17, 2136-2142 (1997).
27. D. X. Tishkoff, Proc. Natl. Acad. Sci. U.S.A 94, 7487-7492 (1997).
28. A. E. Coluccio, PLoS One 3, e2873 (2008).
29. D. A. Treco, Curr. Protoc. Mol. Biol. Chapter 13, Unit 13 12 (2008).
30. R. A. Reenan, Genetics 132, 975-985 (1992).
31. D. E. Lea, J. Genet. 49, 264 (1949).
32. L. Zheng, H. Dai, M. L. Hegde, M. Zhou, Z. Guo, X. Wu, J. Wu, L. Su, X. Zhong, S. Mitra, Q. Huang, K. H. Kernstine, G. P. Pfeifer, B. Shen, Fen1 mutations that specifically disrupt its interaction with PCNA cause aneuploidy-associated cancer. Cell Res. 21, 1052-1067 (2011).
33. S. Liu, G. Lu, S. Ali, W. Liu, L. Zheng, H. Dai, H. Li, H. Xu, Y. Hua, Y. Zhou, J. Ortega, G. M. Li, T. A. Kunkel, B. Shen, Okazaki fragment maturation involves alpha-segment error editing by the mammalian FEN1/MutSalpha functional complex. EMBO J. 34, 1829-1843 (2015).
34. R. E. Johnson, Proc. Natl. Acad. Sci. U.S.A 109, 1245512460 (2012).
35. R. E. Johnson, Meth. Enzymol. 408, 390-407 (2006).
36. P. M. Burgers, J. Biol. Chem. 273, 19756-19762 (1998).
37. L. Zheng, M. Li, J. Shan, R. Krishnamoorthi, B. Shen, Distinct roles of two Mg2+ binding sites in regulation of murine flap endonuclease-1 activities. Biochemistry 41, 10323-10331 (2002).
38. L. Zheng, M. Zhou, Z. Guo, H. Lu, L. Qian, H. Dai, J. Qiu, E. Yakubovskaya, D. F. Bogenhagen, B. Demple, B. Shen, Human DNA2 is a mitochondrial nuclease/helicase for efficient processing of DNA replication and repair intermediates. Mol. Cell 32, 325-336 (2008).
39. Y. Quan, et al, Cell Rep. 13, 2576-2586 (2015).
40. B. Cao, et al, Nucleic Acids Res. 48, 6715-6725 (2020).
41. A. M. Sriramachandran, et al, Mol. Cell 78, 975-985 e977 (2020).
42. M. D. Robinson, et al, Bioinformatics 26, 139-140 (2010).
43. E. J. Jaehnig, et al, Cell Rep. 4, 174-188 (2013).
44. S. Falcon, et al, Bioinformatics 23, 257-258 (2007).
45. D. C. Koboldt, et al, Bioinformatics 25, 2283-2285 (2009).
46. K. Ye, et al, Bioinformatics 25, 2865-2871 (2009).
47. Z. Gu, et al, Nat Genet. 51, 296-307 (2019).
48. H. Fairfield, et al, Genome Biol. 12, R86 (2011).
49. W. J. Kent, Genome Res. 12, 656-664 (2002).
50. L. Zheng, J. Jia, L. D. Finger, Z. Guo, C. Zer, B. Shen, Functional regulation of FEN1 nuclease and its link to cancer. Nucleic Acids Res. 39, 781-794 (2011).
51. J. R. Jackson, et al, Cancer Res. 60, 566-572 (2000).

What is claimed is:

1. A method for detecting a 3′ flap Okazaki fragment in a patient having cancer, the method comprising detecting a 3′ flap Okazaki fragment in a biological sample obtained from the patient having cancer.

2. The method of claim 1, wherein the biological sample contains an elevated level of the 3′ flap Okazaki fragment relative to a control.

3. The method of claim 1, further comprising administering to the patient a therapeutically effective amount of a DNA Damage Response Inhibitor.

4. A method for treating cancer in a patient in need thereof, the method comprising:
(i) detecting a 3′ flap Okazaki fragment in a biological sample obtained from the patient; and
(ii) administering to the patient a therapeutically effective amount of a DNA Damage Response Inhibitor.

5. A method for treating cancer in a patient in need thereof, the method comprising administering to the patient a therapeutically effective amount of a DNA Damage Response Inhibitor; wherein a biological sample obtained from the patient contains a 3′ flap Okazaki fragment.

6. The method of claim 1, wherein the biological sample is a cancer cell.

7. The method of claim 1, wherein the biological sample is genomic DNA in a cancer cell.

8. The method of claim 1, wherein the cancer is leukemia.

9. The method of claim 1, wherein the cancer is acute lymphoblastic leukemia.

10. The method of claim 1, wherein the cancer is lung cancer.

11. The method of claim 10, wherein the lung cancer is EGFR-mutated lung cancer.

12. The method of claim 10, wherein the lung cancer is small cell lung cancer.

13. The method of claim 10, wherein the lung cancer is EGFR-mutated small cell lung cancer.

14. The method of claim 10, wherein the lung cancer is non-small cell lung cancer.

15. The method of claim 10, wherein the lung cancer is EGFR-mutated non-small cell lung cancer.

16. The method of claim 3, wherein the DNA Damage Response Inhibitor is an ATM kinase inhibitor, an ATR kinase inhibitor, a Chk1 kinase inhibitor, a Chk2 kinase inhibitor, or a combination of two or more thereof.

17. The method of claim 16, wherein the ATM kinase inhibitor is AZD0156, KU-60019, or AZD1390.

18. The method of claim 16, wherein the ATR kinase inhibitor is berzosertib or elimusertib.

19. The method of claim 16, wherein the Chk1 kinase inhibitor is SRA737 or prexasertib.

20. The method of claim 1, further comprising administering to the patient a therapeutically effective amount of an anticancer agent, a therapeutically effective amount of radiation therapy, or a combination thereof.
